(19) United States
(12) Patent Application Publication
(10) Pub. No.: US 2009/0054440 A1
(43) Pub. Date: Feb. 26, 2009

(54) Quinoline derivatives as neurokinin receptor antagonists

(76) Inventors: William Robert Carling, Bishop's Stortford (GB); Jason Matthew Elliott, Felsted (GB); Elena Mezzogori, Ravenna (IT); Michael Geoffrey Neil Russell, Hertfordshire (GB); Brian John Williams, Great Dunmow (GB)

Correspondence Address: MERCK AND CO., INC, P O BOX 2000, RAHWAY, NJ, 07065-0907, US

(21) Appl. No.: 11/920,061

(22) PCT Filed: May 5, 2006
(86) PCT No.: PCT/GB2006/050092

§ 371 Date: Nov. 7, 2007

(30) Foreign Application Priority Data
May 10, 2005 (GB) 0509405.7

(51) Int. Cl. A61K 31/4985; A61K 31/496; C07D 401/06; A61K 31/47; A61P 25/24; C07D 487/04; C07D 215/52; A61K 31/4709

(52) U.S. Cl. 514/249; 544/363; 514/253.06; 514/311; 546/169; 514/314; 544/350

(57) ABSTRACT

The present invention relates to substituted quinoline hydrazides of Formula (I): wherein R¹, R², R³, R⁴, R⁵, X, Y and Z are defined herein, pharmaceutical compositions comprising them and their use in treating diseases mediated by neurokinin-2 and/or neurokinin-3 (NK-3) receptors. These compounds can thus be used in methods of treatment to suppress and treat such disorders.

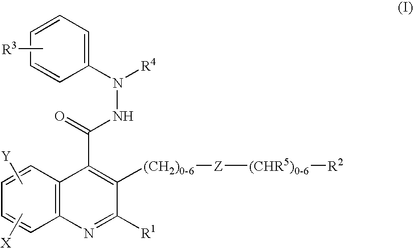

The present invention relates to substituted quinoline-4-carboxylic acid hydrazides defined herein, pharmaceutical compositions comprising them and their use in treating diseases mediated by neurokinin-2 and/or neurokinin-3 (NK-3) receptors. These compounds can thus be used in methods of treatment to suppress and treat such disorders.

Background information on NK-3 receptor antagonists can be found in literature reviews such as Giardina and Raveglia, Exp. Opin. Ther. Patents (1997) 2(4): 307-323 and Giardina et al., Exp. Opin. Ther. Patents (2000) 10(6): 939-960. These references also contain pertinent information on preclinical validation of therapies that can be treated with NK-3 antagonists.

Published International application WO2005/000247 (SmithKline Beecham Corporation) discloses the compounds of formula (A):

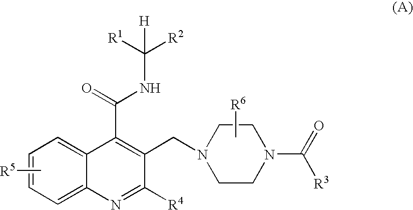

where R¹, R², R³, R⁴, R⁵ and R⁶ are defined therein, as NK2 and NK3 receptor antagonists useful in the treatment of respiratory diseases.

Published International application WO2004/072045 (Merck Sharp & Dohme Ltd) discloses the compounds of formula (B):

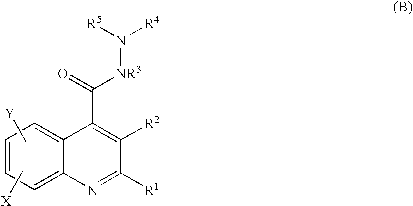

where R¹, R², R³, R⁴, R⁵, X and Y are defined therein, as NK2 and NK3 receptor antagonists useful in the treatment of schizophrenia, COPD, asthma and irritable bowel syndrome.

The present invention thus provides a compound of Formula (I):

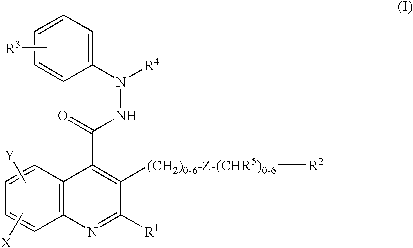

wherein:

R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulfur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;

or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a}, where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen;

or, when R¹ is CONR^{a}R^{b} or SO₂NR^{a}R^{b}, R^{a}, R^{b} and the nitrogen atom to which they are attached form a piperidine, piperazine, pyrrolidine, morpholine, aziridine, azetidine or azepine ring, optionally substituted by 1, 2 or 3 groups independently chosen from hydroxy, C₁₋₄alkyl or C₁₋₄alkoxy;

R² is hydrogen, hydroxy, halogen, CN or CO₂H;

or R² is C₁₋₆alkyl, C₂₋₆alkenyl, C₂₋₆alkynyl, C₃₋₈cycloalkyl, C₁₋₆alkoxy, C(O)OC₁₋₆alkyl, Het, heteroaryl or aryl, optionally substituted by C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, OHet, C(O)C₁₋₆alkyl, C(O)OC₁₋₆alkyl, S(O)₂C₁₋₆alkyl, hydroxy or one to eight halogen atoms, where OHet is optionally substituted by C₁₋₄alkyl;

or R² is NR^{d}R^{e},

where R^{d} and R^{e} are independently selected from hydrogen, C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl and S(O)₂C₁₋₆alkyl, optionally substituted by NR^{f}R^{g} halogen, C₁₋₆alkyl, hydroxy, C₁₋₆alkoxy, CN or CO₂H, where R^{f} and R^{g} are independently selected from hydrogen and C₁₋₆alkyl,
or R^{d} and R^{e}, together with the nitrogen atom to which they are attached, form a nitrogen-containing 3- to 7-membered heterocycle optionally containing a further nitrogen, oxygen or sulfur atom or a S(O) or S(O)₂ group;
which heterocycle is optionally substituted by 1, 2 or 3 groups independently chosen from C₁₋₆alkyl, oxo, halogen and (CH₂)₀₋₆R^{h}, where R^{h} is hydroxy, C₁₋₆alkoxy, C₃₋₈cycloalkyl, bicyclo[3.3.1]non-9-yl, C(O)Rⁱ, C(O)ORⁱ, NRⁱR^{j}, CONRⁱR^{j}, Het, heteroaryl, aryl, SO₂Het, SO₂heteroaryl, SO₂(CH₂)₀₋₃aryl or SO₂C₁₋₆alkyl, where C₁₋₆alkyl and C₃₋₈cycloalkyl, either as separate moieties or as part of a moiety, and heteroaryl, Het and aryl are optionally substituted by 1 to 8 halogen atoms, hydroxy, methoxy, oxo, C₁₋₄alkyl, CF₃ or CH₂CF₃, and C₃₋₈cycloalkyl is optionally fused or spiro-fused to Het, heteroaryl or aryl, where Het and aryl, either as separate moieties or as part of a moiety, are optionally substituted by C₁₋₆alkyl, hydroxy or halogen, and optionally fused to Het, heteroaryl, aryl or C₃₋₈cycloalkyl, and where Rⁱ and R^{j} are independently selected from hydrogen, C₁₋₆alkyl, (CH₂)₀₋₃aryl, (CH₂)₀₋₃heteroaryl, (CH₂)₀₋₃Het and C(O)C₁₋₆ alkyl;
which heterocycle is further optionally fused or spiro-fused to Het, heteroaryl, aryl or C₃₋₈cycloalkyl, optionally substituted by C₁₋₆alkyl, C₃₋₈cycloalkyl, hydroxy, C₁₋₆alkoxy, halogen, oxo, C(O)OC₁₋₆alkyl, Het, CF₃, CHF₂ or CH₂CF₃;
which heterocycle is further optionally bridged by —(CH₂)₁₋₂—;

R³ is hydrogen, hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, optionally substituted by 1 to 8 halogen atoms;

R⁴ is hydrogen, C₁₋₆alkyl, C(O)C₁₋₆alkyl, C(O)OC₁₋₆alkyl, (CH₂)₀₋₃phenyl or heteroaryl, optionally substituted by 1 to 3 halogen atoms;

R⁵ is hydrogen, C₁₋₆alkyl, hydroxy or C₁₋₆alkoxy;

X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NR^{k}R^{m}, CO₂R^{k} and (CH₂)₀₋₄NRⁿR^{p}, SO₂R^{k}, SO₂NR^{k}R^{m}, optionally substituted by 1 to 8 halogen atoms;

R^{k} and R^{m} are independently chosen from hydrogen, C₁₋₆alkyl, C₂₋₆alkenyl, C₂₋₆alkynyl, C₃₋₈cycloalkyl and (CH₂)₀₋₄aryl;
Rⁿ and R^{p} are independently chosen from hydrogen, C₁₋₆alkyl, C₂₋₆alkenyl, C₂₋₆alkynyl, C₃₋₈cycloalkyl, aryl, C(O)R^{q}, COOR^{q} and S(O)₂R^{q};
R^{q} is hydrogen, C₁₋₆alkyl, C₂₋₆alkenyl, C₂₋₆alkynyl, C₃₋₈cycloalkyl or (CH₂)₀₋₄aryl;

Z is a bond, O, S, SO, SO₂, CO, NR^{c}SO₂ or SO₂NR^{c}, NR^{c}, NR^{c}CO or CONR^{c}, where R^{c} is hydrogen or C₁₋₆alkyl, optionally substituted by one to four halogen atoms;

or a pharmaceutically acceptable salt thereof.

In one embodiment of the present invention, there is provided a compound of Formula (Io):

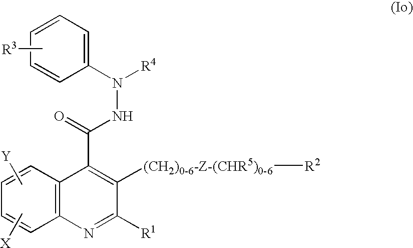

wherein:

R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulfur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;

or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a}, where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen;

or, when R¹ is CONR^{a}R^{b} or SO₂NR^{a}R^{b}, R^{a}, R^{b} and the nitrogen atom to which they are attached form a piperidine, piperazine, pyrrolidine, morpholine, aziridine, azetidine or azepine ring, optionally substituted by 1, 2 or 3 groups independently chosen from hydroxy, C₁₋₄alkyl or C₁₋₄alkoxy;

R² is hydrogen, hydroxy, halogen, CN or CO₂H;

or R² is C₁₋₆alkyl, C₂₋₆alkenyl, C₂₋₆alkynyl, C₃₋₈cycloalkyl, C₁₋₆alkoxy, C(O)OC₁₋₆alkyl, Het, heteroaryl or aryl, optionally substituted by C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl, S(O)₂C₁₋₆alkyl or one to eight halogen atoms;

or R² is NR^{d}R^{e},

where R^{d} and R^{e} are independently selected from hydrogen, C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl and S(O)₂C₁₋₆alkyl, optionally substituted by NR^{f}R^{g}, halogen, C₁₋₆alkyl, hydroxy, C₁₋₆alkoxy, CN or CO₂H, where R^{f} and R^{g} are independently selected from hydrogen and C₁₋₆alkyl, or R^{d} and R^{e}, together with the nitrogen atom to which they are attached, form a saturated nitrogen-containing 3- to 7-membered heterocycle optionally containing a further nitrogen, oxygen or sulfur atom or a S(O) or S(O)₂ group;
which heterocycle is optionally substituted by 1, 2 or 3 groups independently chosen from C₁₋₆alkyl, oxo, halogen and (CH₂)₀₋₆R^{h}, where R^{h} is hydroxy, C₁₋₆alkoxy, C₃₋₈cycloalkyl, C(O)Rⁱ, C(O)ORⁱ, NRⁱR^{j}, CONRⁱR^{j}, Het, heteroaryl, aryl, SO₂Het, SO₂heteroaryl, SO₂(CH₂)₀₋₃aryl or SO₂C₁₋₆alkyl, where C₁₋₆alkyl and C₃₋₈cycloalkyl, either as separate moieties or as part of a moiety, are optionally substituted by 1 to 8 halogen atoms or C₁₋₄alkyl, and C₃₋₈cycloalkyl is optionally fused or spiro-fused to Het, heteroaryl or aryl, where Het and aryl, either as separate moieties or as part of a moiety, are optionally substituted by C₁₋₆alkyl, hydroxy or halogen, and optionally fused to Het, heteroaryl, aryl or C₃₋₈cycloalkyl, and where Rⁱ and R^{j} are independently selected from hydrogen, C₁₋₆alkyl, (CH₂)₀₋₃aryl, (CH₂)₀₋₃heteroaryl, (CH₂)₀₋₃Het and C(O)C₁₋₆ alkyl;
which heterocycle is further optionally fused or spiro-fused to Het, heteroaryl, aryl or C₃₋₈cycloalkyl, optionally substituted by C₁₋₆alkyl, C₃₋₈cycloalkyl, hydroxy, C₁₋₆alkoxy, halogen, oxo, CF₃, CHF₂ or CH₂CF₃;
which heterocycle is further optionally bridged by —(CH₂)₁₋₂—;

R³ is hydrogen, hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, optionally substituted by 1 to 8 halogen atoms;

R⁴ is hydrogen, C₁₋₆alkyl, C(O)C₁₋₆alkyl, C(O)OC₁₋₆alkyl or (CH₂)₀₋₃phenyl;

R⁵ is hydrogen, C₁₋₆alkyl, hydroxy or C₁₋₆alkoxy;

X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NR^{k}R^{m}, CO₂R^{k} and (CH₂)₀₋₄NRⁿR^{p}, SO₂R^{k}, SO₂NR^{k}R^{m}, optionally substituted by 1 to 8 halogen atoms;

R^{k} and R^{m} are independently chosen from hydrogen, C₁₋₆alkyl, C₂₋₆alkenyl, C₂₋₆alkynyl, C₃₋₈cycloalkyl and (CH₂)₀₋₄aryl;
Rⁿ and R^{p} are independently chosen from hydrogen, C₁₋₆alkyl, C₂₋₆alkenyl, C₂₋₆alkynyl, C₃₋₈cycloalkyl, aryl, C(O)R^{q}, COOR^{q} and S(O)₂R^{q};
R^{q} is hydrogen, C₁₋₆alkyl, C₂₋₆alkenyl, C₂₋₆alkynyl, C₃₋₈cycloalkyl or (CH₂)₀₋₄aryl;

Z is a bond, O, S, SO, SO₂, CO, NR^{c}SO₂ or SO₂NR^{c}, NR^{c}, NR^{c}CO or CONR^{c}, where R^{c} is hydrogen or C₁₋₆alkyl, optionally substituted by one to four halogen atoms;

or a pharmaceutically acceptable salt thereof.

In one embodiment of the invention, R¹ is aryl or heteroaryl, optionally substituted by 1, 2 or 3 groups independently chosen from hydroxy, halogen, nitro, cyano, amino, CF₃, C₁₋₄alkyl, C₂₋₄alkenyl or C₂₋₄alkynyl. Preferably, R¹ is aryl, optionally substituted by hydroxy, halogen or CF₃. More preferably, R¹ is phenyl, optionally substituted by hydroxy, halogen or CF₃. Most preferably, R¹ is phenyl.

In another embodiment of the invention, R² is CN, CO₂H, C(O)OC₁₋₆alkyl, Het, heteroaryl, aryl or NR^{d}R^{e}, where Het, heteroaryl and aryl are optionally substituted by C₁₋₆alkyl C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl, S(O)₂C₁₋₆alkyl or one to eight halogen atoms, and where NR^{d}R^{e} is as hereinbefore defined.

Preferably, R² is CN, CO₂H, C(O)OCH₃, piperidinyl, phenyl, pyrrolyl, pyrazolyl, imidazolyl, triazolyl, tetrazolyl or NR^{d}R^{e}, where piperidinyl, phenyl, pyrrolyl, pyrazolyl, imidazolyl, triazolyl and tetrazolyl are optionally substituted by C₁₋₆alkyl, and where NR^{d}R^{e} is as hereinbefore defined.

More preferably, R² is CN, CO₂H, C(O)OCH₃, phenyl, imidazolyl, triazolyl or NR^{d}R^{e}, where imidazolyl and triazolyl are optionally substituted by methyl or ethyl, and where NR^{d}R^{e} is as hereinbefore defined.

Most preferably, R² is CN, CO₂H, C(O)OCH₃, phenyl, 1-methyl-1H-imidazol-2-yl, 1-methyl-1H-1,2,4,-triazol-3-yl, 1-methyl-1H-1,2,4-triazol-5-yl or NR^{d}R^{e}, where NR^{d}R^{e} is as hereinbefore defined.

When R² is NR^{d}R^{e}, preferably R^{d} and R^{e} are independently selected from C₁₋₆alkyl, (CH₂)₀₋₃aryl, (CH₂)₀₋₃heteroaryl and (CH₂)₀₋₃Het, optionally substituted by N(C₁₋₆alkyl)₂, C₁₋₆alkyl, hydroxy, CN or CO₂H,

or R^{d} and R^{e}, together with the nitrogen atom to which they are attached, form a saturated nitrogen-containing 5- or 6-membered heterocycle optionally containing a further nitrogen, oxygen or sulfur atom, which heterocycle is optionally substituted by C₁₋₄alkyl, oxo, halogen or (CH₂)₀₋₆R^{h},
where R^{h} is as hereinbefore defined,
which heterocycle is further optionally fused or spiro-fused to Het, heteroaryl or aryl, optionally substituted by hydroxy, halogen, oxo or CF₃,
which heterocycle is further optionally bridged by —(CH₂)₁₋₂—.

More preferably, R^{d} and R^{e} are independently selected from C₁₋₄alkyl, (CH₂)₀₋₂aryl, (CH₂)₀₋₂heteroaryl and Het, optionally substituted by N(C₁₋₄alkyl)₂, C₁₋₄alkyl, hydroxy, CN or CO₂H, or R^{d} and R^{e}, together with the nitrogen atom to which they are attached, form a saturated nitrogen-containing 5- or 6-membered heterocycle optionally containing a further nitrogen or sulfur atom, which heterocycle is optionally substituted by methyl, ethyl, propyl, oxo, chlorine, fluorine or (CH₂)₀₋₆R^{h}, where R^{h} is as hereinbefore defined, which heterocycle is further optionally fused or spiro-fused to pyrrolidinyl, tetrahydrofuranyl, imidazolidinyl, imidazolyl, triazolyl or phenyl, optionally substituted by halogen, oxo or CF₃.

Most preferably, R^{d} and R^{e} are independently selected from methyl, ethyl, propyl, butyl, (CH₂)₁₋₂phenyl, (CH₂)thienyl, (CH₂)₁₋₂pyridyl and piperidine, optionally substituted by N(CH₃)₂, methyl, hydroxy, CN or CO₂H,

or R^{d} and R^{e}, together with the nitrogen atom to which they are attached, form a piperidinyl, piperazinyl, thiazolidinyl or thiomorpholinyl ring,
optionally substituted by methyl, ethyl, isopropyl, oxo, fluorine or (CH₂)₀₋₆R^{h}, where R^{h} is as hereinbefore defined, which heterocycle is further optionally fused or spiro-fused to pyrrolidinyl, tetrahydrofuranyl, imidazolidinyl, imidazolyl, triazolyl or phenyl, optionally substituted by oxo or CF₃.

Especially, R^{d} is methyl, ethyl, propyl or butyl, and R^{e} is methyl, ethyl, propyl, (CH₂)phenyl, (CH₂)₂phenyl, (CH₂)thienyl, (CH₂)pyridyl, (CH₂)₂pyridyl and piperidinyl, optionally substituted by N(CH₃)₂, methyl, hydroxy, CN or CO₂H,

or R^{d} and R^{e}, together with the nitrogen atom to which they are attached, form a piperidin-1-yl, piperazin-1-yl, 1,3-thiazolidin-3-yl or thiomorpholin-4-yl ring,
optionally substituted by methyl, ethyl, isopropyl, oxo, fluorine or (CH₂)₀₋₆R^{h}, where R^{h} is as hereinbefore defined,
which heterocycle is further optionally fused to pyrrolidinyl, 4-trifluoromethylimidazolyl, 1,2,4-triazolyl, 3-trifluoromethyl-1,2,4-triazolyl or phenyl,
or further optionally spiro-fused to 2-oxotetrahydrofuranyl or 2-oxoimidazolidinyl.

More especially, R^{d} is methyl, ethyl, n-propyl or n-butyl and R^{e} is (CH₂)₂OH, (CH₂)CN, (CH₂)₂CN, (CH₂)CH(OH)CH₂OH, (CH₂)phenyl, CH(CO₂H)phenyl, CH₂CH(OH)phenyl, (CH₂)₂N(CH₃)₂, (CH₂)₃N(CH₃)₂, (CH₂)thienyl, (CH₂)pyridyl, (CH₂)₂pyridyl or 1-methylpiperidin-4-yl,

or R^{d} and R^{e}, together with the nitrogen atom to which they are attached, form a piperidin-1-yl, piperazin-1-yl, 1,3-thiazolidin-3-yl or thiomorpholin-4-yl, 3-(hexahydropyrrolo[1,2-a]pyrazin-2(1H)-yl), 2-(trifluoromethyl)-5,6-dihydroimidazo[1,2-a]pyrazin-7(8H)-yl, 3-(5,6-dihydro[1,2,4]triazolo[4,3-a]pyrazin-7(8H)-yl), 3-(trifluoromethyl)-5,6-dihydro[1,2,4]triazolo[4,3-a]pyrazin-7(8H)-yl, 2-(trifluoromethyl)-5,6-dihydro[1,2,4]triazolo[1,5-a]pyrazin-7(8H)-yl, or 3,4-dihydroisoquinolin-2-(1H)-yl ring, where the piperidin-1-yl and piperazin-1-yl rings are optionally substituted by methyl, ethyl, isopropyl, oxo, fluorine or (CH₂)₀₋₆R^{h}, where R^{h} is as hereinbefore defined, which heterocycle is further optionally spiro-fused to 2-oxotetrahydrofuranyl or 2-oxoimidazolidinyl.

When R^{d} and R^{e}, together with the nitrogen atom to which they are attached, form a heterocycle as hereinbefore defined, and that heterocycle is substituted by (CH₂)₀₋₆R^{h}, preferably R^{h} is hydroxy, C₁₋₄alkoxy, C₃₋₆cycloalkyl, C(O)Rⁱ, C(O)ORⁱ, NRⁱR^{j}, CONRⁱR^{j}, Het, heteroaryl, aryl, SO₂Het, SO₂heteroaryl, SO₂(CH₂)₀₋₁aryl or SO₂C₁₋₄alkyl, where C₁₋₄ alkyl, either as a separate moiety or as part of a moiety, is optionally substituted by 1 to 4 halogen atoms, where Het and aryl, either as separate moieties or as part of a moiety, are optionally substituted by C₁₋₄alkyl, hydroxy or halogen, and optionally fused to Het, and where Rⁱ and R^{j} are as hereinbefore defined.

In another embodiment of the invention, R³ is hydrogen, hydroxy, halogen, C₁₋₄alkyl, C₂₋₄alkenyl or C₂₋₄alkynyl, optionally substituted by 1 to 8 halogen atoms. Preferably, R³ is hydrogen, halogen, C₁₋₄alkyl, C₂₋₄alkenyl or C₂₋₄alkynyl. More preferably, R³ is hydrogen, halogen or C₁₋₄ alkyl. Most preferably, R³ is hydrogen.

In another embodiment of the invention, R⁴ is C(O)C₁₋₆alkyl, C(O)OC₁₋₆alkyl, phenyl or benzyl. Preferably, R⁴ is C(O)CH₂CH₃, C(O)OCH₃, C(O)OCH₂CH₃, phenyl or benzyl. More preferably, R⁴ is C(O)OCH₃.

In another embodiment of the invention, R⁵ is hydrogen, C₁₋₄alkyl or hydroxy. More preferably, R⁵ is hydrogen or hydroxy. Most preferably, R⁵ is hydrogen.

In another embodiment of the invention, X is hydrogen, hydroxy, nitro, cyano, CF₃, halogen, C₁₋₆alkyl, C₂₋₆alkenyl, C₂₋₆alkynyl, C₃₋₈cycloalkyl or C₁₋₆alkoxy. Preferably, X is hydrogen, halogen, C₁₋₆alkyl or C₂₋₆alkenyl. More preferably, X is hydrogen or halogen. Most preferably, X is hydrogen.

In another embodiment of the invention, Y is hydrogen, hydroxy, nitro, cyano, CF₃, halogen, C₁₋₆alkyl, C₂₋₆alkenyl, C₂₋₆alkynyl, C₃₋₈cycloalkyl or C₁₋₆alkoxy. Preferably, Y is hydrogen or halogen, C₁₋₆alkyl or C₂₋₆alkenyl. More preferably, Y is hydrogen or halogen. Most preferably, Y is hydrogen.

In another embodiment of the invention, Z is a bond, O or S. More preferably, Z is a bond or O.

In one embodiment of the present invention, there is provided the compound of formula (Ia):

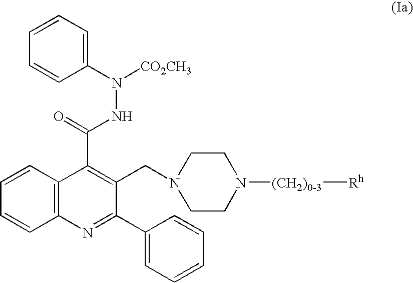

or a pharmaceutically acceptable salt thereof,
wherein R^{h} is as defined in relation to formula (I),
or wherein (CH₂)₀₋₃R^{h} is C₁₋₆alkyl.

Preferably, R^{h} is t-butyl, hydroxy, trifluoromethyl, C₃₋₆cycloalkyl, C(O)Rⁱ, C(O)ORⁱ, CONRⁱR^{j}, Het, heteroaryl, aryl, SO₂heteroaryl, SO₂(CH₂)₀₋₃aryl or SO₂C₁₋₄alkyl,

where C₃₋₆cycloalkyl is optionally substituted by C₁₋₄alkyl and optionally spiro-fused to Het,
where SO₂C₁₋₄alkyl is optionally substituted by 1 to 6 halogen atoms,
where Het and aryl, either as separate moieties or as part of a moiety, are optionally substituted by C₁₋₄alkyl hydroxy or methoxy,
and where Rⁱ and R^{j} are independently selected from C₁₋₄alkyl, (CH₂)₀₋₁aryl, heteroaryl and Het, or (CH₂)₀₋₃R^{h} is methyl, ethyl or isopropyl.

Examples of suitable R^{h} groups are t-butyl, hydroxy, cyclopropyl, cyclopentyl, cyclohexyl, methylcyclohexyl, 1,4-dioxaspiro[4.5]dec-8-yl, pyridyl, pyrimidyl, tetrahydropyranyl, phenyl, hydroxyphenyl, C(O)CH₃, C(O)CH₂CH₃, C(O)CH(CH₃)₂, C(O)C(CH₃)₃, C(O)phenyl, C(O)CH₂phenyl, C(O)N(CH₃)₂, C(O)morpholinyl, C(O)furanyl, C(O)OCH₃, C(O)OC(CH₃)₃, C(O)OCH₂CH(CH₃)₂, C(O)CH₂phenyl, SO₂CH₃, SO₂CH₂CH₃, SO₂CH₂CH₂CH₃, SO₂phenyl, SO₂CH₂phenyl, SO₂CF₃, SO₂pyridyl, SO₂thienyl, SO₂(methylpyrazolyl), trifluoromethyl, dimethylcyclopentyl, methyltetrahydrofuranyl, methyltetrahydropyranyl, thiane, thianedioxide, methylthiazole, methylfurazan and methoxypyrimidine.

In another embodiment of the present invention, there is provided the compound of formula (Ib):

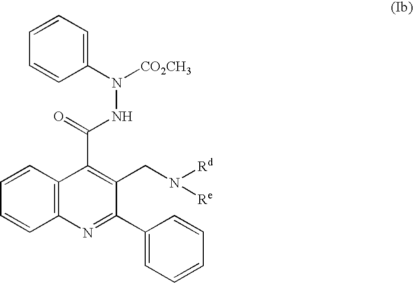

or a pharmaceutically acceptable salt thereof,
where R^{d} and R^{e} are as defined in relation to formula (I).

Preferably, R^{d} and R^{e}, together with the nitrogen atom to which they are attached, form a saturated nitrogen-containing 5- or 6-membered heterocycle optionally containing a further nitrogen, oxygen or sulfur atom, which heterocycle is optionally substituted by C₁₋₄alkyl, oxo, halogen, or (CH₂)₀₋₆R^{h} where R^{h} is as defined in relation to formula (I), which heterocycle is optionally fused or spiro-fused to Het, heteroaryl or aryl, optionally substituted by hydroxy, oxo, halogen or CF₃.

More preferably, R^{d} and R^{e}, together with the nitrogen atom to which they are attached, form a pyrrolidinyl, piperidinyl, substituted piperazinyl, thiazolidinyl or thiomorpholinyl heterocycle, which heterocycle is optionally substituted by methyl, oxo, fluorine or (CH₂)₀₋₆R^{h}, where R^{h} is hydroxy, methoxy, C(O)Rⁱ, C(O)ORⁱ, NRⁱR^{j}, CONRⁱR^{j}, Het, heteroaryl or aryl, where Rⁱ and R^{j} are as defined in relation to formula (I), which heterocycle is optionally fused to Het, heteroaryl or aryl, optionally substituted by CF₃, which heterocycle is further optionally spiro-fused to Het, optionally substituted by hydroxy or oxo.

Examples of suitable groups where R^{d} and R^{e}, together with the nitrogen atom to which they are attached, form a heterocycle as hereinbefore described are thiazolidin-3-yl, thiomorpholin-4-yl, 3-hydroxypyrrolidin-1-yl, 2-(hydroxymethyl)pyrrolidin-1-yl, 2-(methoxymethyl)pyrrolidin-1-yl, 2-carboxypyrrolidin-1-yl, 2-(aminocarbonyl)pyrrolidin-1-yl, 2-[(dimethylamino)carbonyl]pyrrolidin-1-yl, 2-pyridin-3-ylpyrrolidin-1-yl, 3-[acetyl(methyl)amino]pyrrolidin-1-yl, 4,4-difluoropiperidin-1-yl, 3-hydroxypiperidin-1-yl, 4-hydroxypiperidin-1-yl, 2-(hydroxymethyl)piperidin-1-yl, 3-carboxypiperidin-1-yl, 4-carboxypiperidin-1-yl, 3-(aminocarbonyl)piperidin-1-yl, 4-(aminocarbonyl)piperidin-1-yl, 4-(ethoxycarbonyl)piperidin-1-yl, 4-pyrrolidin-1-ylpiperidin-1-yl, 1,4′-bipiperidin-1′-yl, 3-(piperidin-1-ylmethyl)piperidin-1-yl, 3-(3-ethyl-1,2,4-oxadiazol-5-yl)piperidin-1-yl, 3-(4-methyl-4H-1,2,4-triazol-3-yl)piperidin-1-yl, 4-(2H-tetrazol-2-yl)piperidin-1-yl, 4-(2-ethyl-4,5,6,7-tetrahydropyrazolo[1,5-a]pyridine-3-yl)piperidin-1-yl, 3,4-dihydroisoquinolin-2(1H)-yl, 2-oxo-1,3,8-triazaspiro[4.5]dec-8-yl, 1-oxo-2-oxa-8-azaspiro[4.5]dec-8-yl, 2-oxo-piperazin-1-yl, 3-oxo-piperazin-1-yl, 2-oxo-4-tertbutyloxycarbonylpiperazin-1-yl, 3-oxo-4-methylpiperazin-1-yl, 3-oxo-4-pyridin-4-ylpiperazin-1-yl, 4-[(1-methyl-1H-1,2,4-triazol-5-yl)methyl]-3-oxopiperazin-1-yl, hexahydropyrrolo[1,2-a]pyrazin-2(1H)-yl, 5,6-dihydro[1,2,4]triazolo[4,3-a]pyrazin-7(8H)-yl, 3-(trifluoromethyl)-5,6-dihydro[1,2,4]triazolo[4,3-a]pyrazin-7(8H)-yl, 2-(trifluoromethyl)-5,6-dihydro[1,2,4]triazolo[1,5-a]pyrazin-7(8H)-yl, and 2-(trifluoromethyl-5,6-dihydroimidazo[1,2-a]pyrazin-7(8H)-yl.

Further examples of suitable groups where R^{d} and R^{e}, together with the nitrogen atom to which they are attached, form a heterocycle as herinbefore described are:

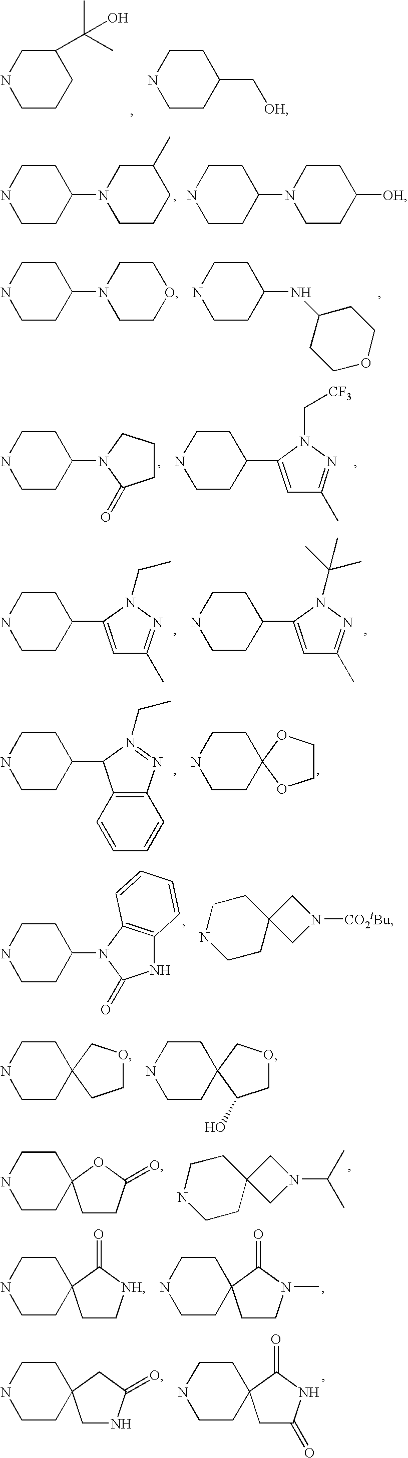

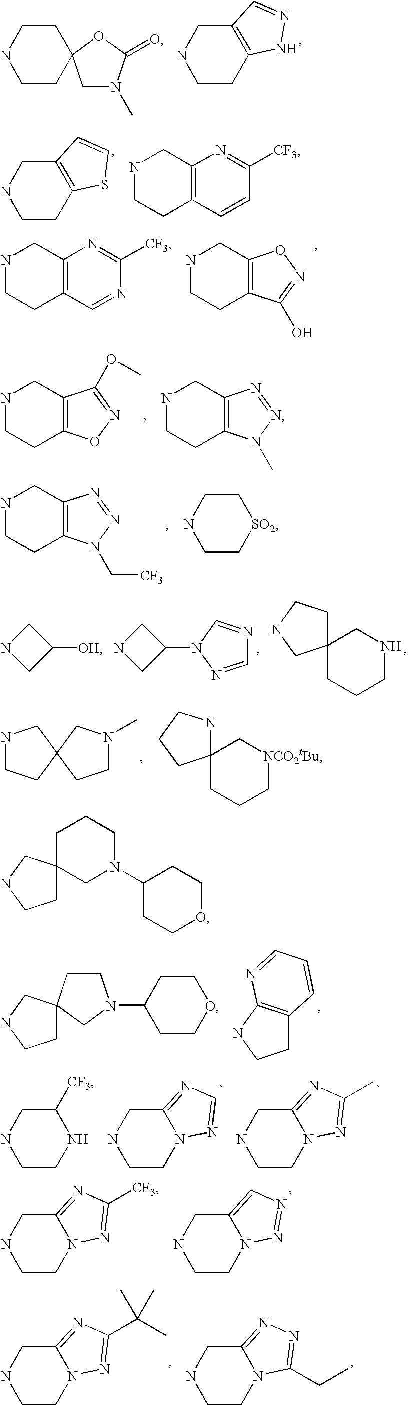

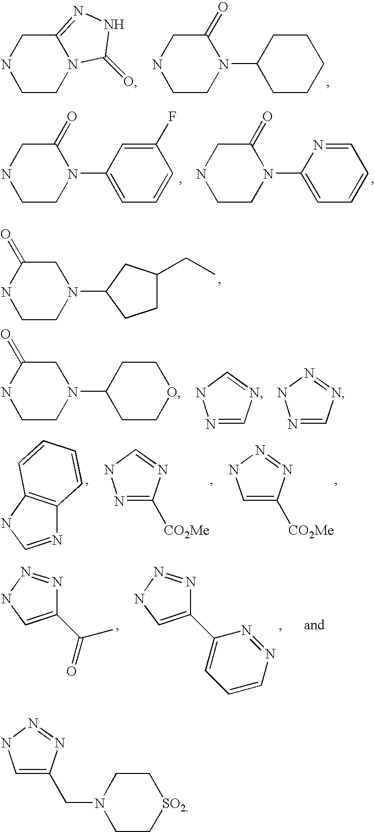

Also, preferably, R^{d} and R^{e} are independently selected from C₁₋₆alkyl, (CH₂)₀₋₃aryl, (CH₂)₀₋₃heteroaryl and (CH₂)₀₋₃Het, optionally substituted by N(C₁₋₆alkyl)₂, halogen, C₁₋₆alkyl, hydroxy, CN or CO₂H.

More preferably, R^{d} and R^{e} are independently selected from C₁₋₄alkyl, (CH₂)₁₋₂aryl, (CH₂)₁₋₂ heteroaryl and Het, optionally substituted by N(C₁₋₄alkyl)₂, C₁₋₄alkyl, hydroxy, CN or CO₂H.

Most preferably, R^{d} is methyl, ethyl, propyl, butyl or cyanoethyl and R^{e} is C₁₋₃alkyl, (CH₂)₁₋₂phenyl, CH₂thienyl, (CH₂)₁₋₂pyridyl or piperidinyl, optionally substituted by N(CH₃)₂, methyl, hydroxy, CN or CO₂H.

Examples of suitable R^{d} groups are methyl, ethyl, n-butyl and cyanoethyl.
Examples of suitable R^{e} groups are cyanomethyl, hydroxyethyl, cyanoethyl, dimethylaminoethyl, dimethylaminopropyl, 2,3-dihydroxypropyl, phenylmethyl, carboxy(phenyl)methyl, 2-hydroxy-2-phenylethyl, 2-thienylmethyl, 3-pyridylmethyl, 4-pyridylmethyl, 2-pyridylethyl and 1-methylpiperidin-4-yl.

In another embodiment of the present invention, there is provided the compound of formula (Ic):

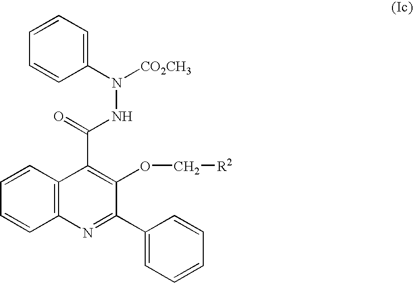

or a pharmaceutically acceptable salt thereof,
wherein R² is as defined in relation to formula (I).

Preferably, R² is C₁₋₆alkyl, CN, CO₂H, C(O)OC₁₋₆alkyl, aryl, heteroaryl or OHet, optionally substituted by C₁₋₄alkyl, hydroxy or halogen. More preferably, R² is (CH₂)₁₋₄OH, (CH₂)₁₋₄Br, CN, CO₂H, C(O)OCH₃, phenyl, imidazolyl, triazolyl or O-piperidinyl, optionally substituted by methyl, ethyl or ^{t}butyl. Most preferably, R² is CN, CO₂H, C(O)OCH₃, phenyl, 1-methyl-1H-imidazol-2-yl, 1-methyl-1H-1,2,4-triazol-5-yl, 1-methyl-1H-1,2,4-triazol-3-yl, 1-hydroxyethyl. 1-bromoethyl or

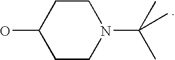

In another embodiment of the present invention, there is provided the compound of formula (Id):

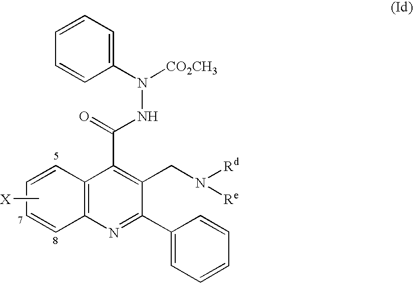

or a pharmaceutically acceptable salt thereof,

wherein X is selected from NO₂, CN, CF₃ and halogen,
and R^{d} and R^{e} are defined in relation to formula (I).

Preferably, X is CN or halogen. More preferably, X is CN, fluorine or bromine.

Preferably, X is at the 5-, 7- or 8-position of the quinoline system.

Preferably, R^{d} and R^{e}, together with the nitrogen atom to which they are attached, form a saturated nitrogen-containing 6-membered heterocycle optionally containing a further nitrogen atom, which heterocycle is optionally substituted by C₁₋₄alkyl, C₃₋₈cycloalkyl, bicyclo[3.3.1]non-9-yl or Het, which optional substituent is optionally substituted by 1 to 8 halogen atoms or C₁₋₄alkyl;

which heterocycle is further optionally fused to Het or heteroaryl, optionally substituted by hydroxy or oxo;
which heterocycle is further optionally bridged by —CH₂—.

More preferably, R^{d} and R^{e}, together with the nitrogen atom to which they are attached, form a piperazinyl ring, optionally substituted by C₃₋₄alkyl, C₄₋₆cycloalkyl, bicyclo[3.3.1]non-9-yl or tetrahydropyranyl, which optional substituent is optionally substituted by 1 to 3 halogen atoms or C₁₋₂alkyl;

which heterocycle is further optionally fused to morpholinyl, pyrrolidinyl or triazolyl, optionally substituted by hydroxy or oxo;
which heterocycle is further optionally bridged by —CH₂—.

Most preferably, R^{d} and R^{e}, together with the nitrogen atom to which they are attached, form a piperazinyl ring, optionally substituted by ⁱpropyl, ^{t}butyl, cyclohexyl, bicyclo[3.3.1]non-9-yl or 4-tetrahydropyranyl, which optional substituent is optionally substituted by 1 to 3 fluorine atoms or methyl;

which heterocycle is further optionally fused to morpholinyl, pyrrolidinyl or 1,2,4-triazolyl, optionally substituted by hydroxy or oxo;
which heterocycle is further optionally bridged by —CH₂—.

In another embodiment of the present invention, there is provided the compound of formula (Ie):

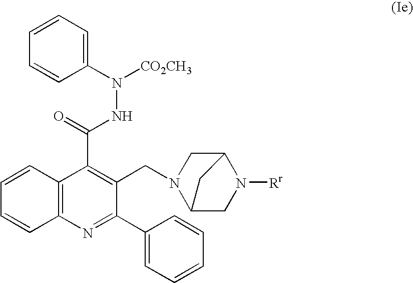

or a pharmaceutically acceptable salt thereof,
wherein R^{r} is hydrogen, C₁₋₆alkyl or (CH₂)₀₋₆R^{h},
where R^{h} is as defined in relation to formula (I), and where C₁₋₆alkyl, C₃₋₈cycloalkyl and heteroaryl are optionally substituted by 1 to 8 halogen atoms or C₁₋₄alkyl.

Preferably, R^{r} is hydrogen, C₁₋₄alkyl or (CH₂)₀₋₁R^{h}, where R^{h} is C₄₋₆cycloalkyl, C(O)Rⁱ, Het or heteroaryl, where C₁₋₄alkyl and heteroaryl are optionally substituted by 1 to 8 fluorine atoms or C₁₋₄alkyl, and where Rⁱ is as defined in relation to formula (I). More preferably, R^{r} is hydrogen, C₁₋₃alkyl, C₄₋₆cycloalkyl, C(O)C₁₋₄alkyl, Het or CH₂heteroaryl, where C₁₋₃alkyl and CH₂heteroaryl are optionally substituted by 1 to 3 fluorine atoms, methyl or butyl. Most preferably, R^{r} is hydrogen, methyl, ⁱpropyl, CH₂CH₂CF₃, cyclohexyl, C(O)C(CH₃)₃, 4-tetrahydropyranyl,

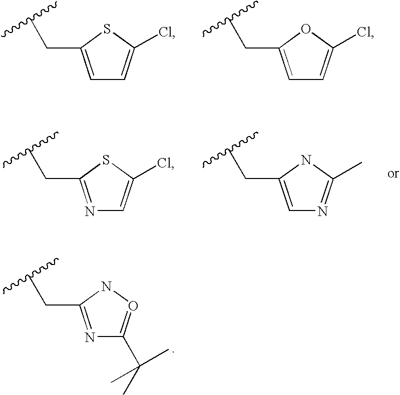

In another embodiment of the present invention, there is provided the compound of formula (If):

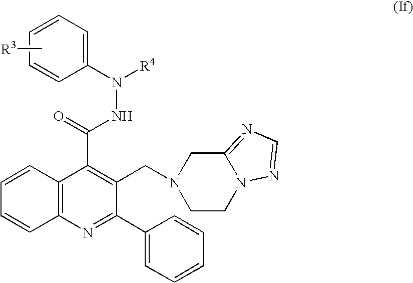

or a pharmaceutically acceptable salt thereof,
wherein R³ and R⁴ are as defined in relation to formula (I).

Preferably, R³ is hydrogen, hydroxy or halogen. More preferably, R³ is hydrogen or fluorine.

Preferably, R⁴ is C₁₋₆alkyl, C(O)OC₁₋₄alkyl, phenyl or heteroaryl, optionally substituted by 1 to 3 halogen atoms. More preferably, R⁴ is C₁₋₄alkyl, C(O)OC₁₋₄alkyl, phenyl, pyridyl or thiazolyl, optionally substituted by 1 to 3 fluorine atoms. Most preferably, R⁴ is ethyl, C(O)OCH₃, phenyl, 3-fluorophenyl, 3-pyridyl or 2-thiazolyl.

In another embodiment of the present invention, there is provided the compound of formula (Ig):

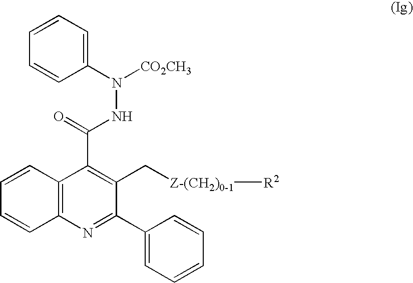

or a pharmaceutically acceptable salt thereof,
wherein Z and R² are as defined in relation to formula (I).

Preferably Z is O, S, SO or SO₂.

Preferably, R² is C₁₋₆alkyl, Het or heteroaryl, optionally substituted by C₁₋₆alkyl or Het. More preferably, R² is C₁₋₄alkyl, pyrrolidinyl, piperidinyl or triazolyl, optionally substituted by C₁₋₄alkyl or tetrahydropyranyl. Most preferably, R² is methyl, 3-pyrrolidinyl, 4-piperidinyl or 5-triazolyl, optionally substituted by ethyl or 4-tetrahydropyranyl.

In another embodiment of the present invention, there is provided the compound of formula (Ih):

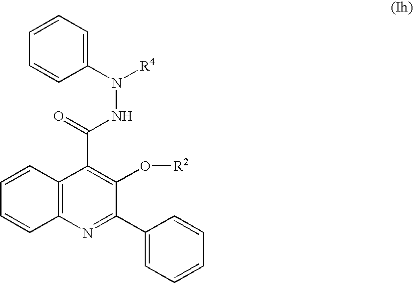

or a pharmaceutically acceptable salt thereof,
wherein R² and R⁴ are as defined in relation to formula (I).

Preferably, R² is C-linked Het, optionally substituted by C₁₋₆alkyl, C(O)OC₁₋₆alkyl or (CH₂)₀₋₃Het. More preferably, R² is C-linked piperidinyl, optionally substituted by C₁₋₄alkyl, C(O)OC₁₋₄alkyl or Het. Most preferably, R² is 3- or 4-piperidinyl, optionally substituted by butyl, C(O)Obutyl or tetrahydropyranyl. Especially, R² is 4-piperidinyl, optionally substituted by ^{t}butyl, C(O)O^{t}butyl or 4-tetrahydropyranyl.

Preferably, R⁴ is hydrogen or C(O)OC₁₋₆alkyl. More preferably, R⁴ is hydrogen or C(O)OC₁₋₄alkyl. Most preferably, R⁴ is hydrogen or C(O)OCH₃.

Particularly preferred compounds of the present invention include those named in the Examples and Tables hereinbelow.

The present invention includes within its scope solvates of the compounds of formula (I), for example hydrates, and salts thereof.

The present invention also includes within its scope any enantiomers, diastereomers, geometric isomers and tautomers of the compounds of formula (I). It is to be understood that all such insomers and mixtures thereof are encompassed within the scope of the invention.

The independent syntheses of any optical isomers or their chromatographic separations may be achieved as known in the art. Their absolute stereochemistry may be determined by the X-ray crystallography of crystalline products or crystalline intermediates which are derivatized, if necessary, with a reagent containing an asymmetric center of known absolute configuration.

As used herein, the term “C₁₋₆alkyl” means linear or branched chain alkyl groups having from 1 to 6 carbon atoms and includes all of the hexyl and pentyl alkyl isomers as well as n-, iso-, sec- and t-butyl, n- and isopropyl, ethyl and methyl. “C₁₋₄alkyl” and “C₁₋₂alkyl” shall be understood in an analogous manner, as shall “C₁₋₆alkoxy” and “C₁₋₄ alkoxy”.

The term “C₂₋₆alkenyl” means linear or branched chain alkenyl groups having from 2 to 6 carbon atoms and includes all of the hexenyl and pentenyl isomers as well as 1-butenyl, 2-butenyl, 3-butenyl, isobutenyl, 1-propenyl, 2-propenyl, and ethenyl (or vinyl).

The term “C₂₋₆alkynyl” means linear or branched chain alkynyl groups having from 2 to 6 carbon atoms and includes all of the hexynyl and pentynyl isomers as well as 1-butynyl, 2-butynyl, 3-butynyl, 1-propynyl, 2-propynyl, and ethynyl (or acetylenyl).

The term “C₃₋₈cycloalkyl” means a cyclic alkane ring having three to eight total carbon atoms (i.e., cyclopropyl, cyclobutyl, cyclopentyl, cyclohexyl, cycloheptyl, or cyclooctyl). The term “C₄₋₇cycloalkyl” refers to a cyclic ring selected from cyclobutyl, cyclopentyl, cyclohexyl and cycloheptyl.

The term “halogen” refers to fluorine, chlorine, bromine and iodine.

The term “aryl” means phenyl or naphthyl.

The term “heteroaryl” means a 5-membered unsaturated ring containing 1, 2, 3 or 4 nitrogen atoms and/or an oxygen or sulfur atom provided no more than two nitrogen atoms are present, or a 6-membered unsaturated ring containing 1, 2 or 3 nitrogen atoms, and includes the following groups: furanyl, imidazolyl, isothiazolyl, isoxazolyl, oxadiazolyl, oxazolyl, pyrazinyl, pyrazolyl, pyridazinyl, pyridazinyl, pyridyl, pyrimidyl, pyrrolyl, tetrazolyl, thiadiazolyl, thiazolyl, thienyl, triazolyl and pyrrolidinyl.

The term “Het” means a heteroaliphatic ring of 3 to 7 ring atoms, which ring contains 1, 2 or 3 heteroatoms selected from N, O or S or a group S(O), S(O)₂, NH or NC₁₋₄alkyl. Examples of Het include aziridinyl, azetidinyl, pyrrolidinyl, piperidinyl, azepinyl, piperazinyl, imidazolidinyl, pyrazolidinyl, tetrahydrofuranyl, dioxolanyl, pyran, dioxanyl, morpholine, oxathiolanyl, dithianyl, oxathianyl, thiomorpholinyl, trioxanyl, trithianyl.

The terms “thiophenyl” and “thienyl” have the same meaning herein and are used interchangeably. Similarly, the following pair of terms has the same meaning: “pyridinyl” and “pyridyl”.

Exemplary compounds of the present invention include those listed in the Examples section and their pharmaceutically acceptable salts.

These compounds and those defined by the immediately preceding definitions are useful in therapy, especially as NK-2 and/or NK-3 antagonists, particularly as NK-3 antagonists.

The terms “administration of” and or “administering a” compound should be understood to mean providing a compound of the invention to the individual in need of treatment.

The term “subject,” (alternatively referred to herein as “patient”) as used herein refers to an animal, preferably a mammal, most preferably a human, who has been the object of treatment, observation or experiment.

The compounds of the present invention may be administered in the form of pharmaceutically acceptable salts. The term “pharmaceutically acceptable salt” is intended to include all acceptable salts such as acetate, lactobionate, benzenesulfonate, laurate, benzoate, malate, bicarbonate, maleate, bisulfate, mandelate, bitartrate, mesylate, borate, methylbromide, bromide, methylnitrate, calcium edetate, methylsulfate, camsylate, mucate, carbonate, napsylate, chloride, nitrate, clavulanate, N-methylglucamine, citrate, ammonium salt, dihydrochloride, oleate, edetate, oxalate, edisylate, pamoate (embonate), estolate, palmitate, esylate, pantothenate, fumarate, phosphate/diphosphate, gluceptate, polygalacturonate, gluconate, salicylate, glutamate, stearate, glycollylarsanilate, sulfate, hexylresorcinate, subacetate, hydrabamine, succinate, hydrobromide, tannate, hydrochloride, tartrate, hydroxynaphthoate, teoclate, iodide, tosylate, isothionate, triethiodide, lactate, panoate, valerate, and the like which can be used as a dosage form for modifying the solubility or hydrolysis characteristics or can be used in sustained release or pro-drug formulations. Depending on the particular functionality of the compound of the present invention, pharmaceutically acceptable salts of the compounds of this invention include those formed from cations such as sodium, potassium, aluminum, calcium, lithium, magnesium, zinc, and from bases such as ammonia, ethylenediamine, N-methyl-glutamine, lysine, arginine, ornithine, choline, N,N′-dibenzylethylene-diamine, chloroprocaine, diethanolamine, procaine, N-benzylphenethyl-amine, diethylamine, piperazine, tris(hydroxymethyl)aminomethane, and tetramethylammonium hydroxide. These salts may be prepared by standard procedures, e.g. by reacting a free acid with a suitable organic or inorganic base. Where a basic group is present, such as amino, an acidic salt, i.e. hydrochloride, hydrobromide, acetate, palmoate, and the like, can be used as the dosage form.

Also, in the case of an alcohol group being present, pharmaceutically acceptable esters can be employed, e.g. acetate, maleate, pivaloyloxymethyl, and the like, and those esters known in the art for modifying solubility or hydrolysis characteristics for use as sustained release or prodrug formulations.

The compounds of the present invention may be administered by oral, parenteral (e.g., intramuscular, intraperitoneal, intravenous, ICV, intracisternal injection or infusion, subcutaneous injection, or implant), by inhalation spray, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. In addition to the treatment of warm-blooded animals such as mice, rats, horses, cattle, sheep, dogs, cats, monkeys, etc., the compounds of the invention are effective for use in humans.

The pharmaceutical compositions for the administration of the compounds of this invention may conveniently be presented in dosage unit form and may be prepared by any of the methods well known in the art of pharmacy. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases. As used herein, the term “composition” is intended to encompass a product comprising the specified ingredients in the specified amounts, as well as any product which results, directly or indirectly, from combination of the specified ingredients in the specified amounts.

The pharmaceutical compositions containing the active ingredient may be in a form suitable for oral use, for example, as tablets, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents, for example, corn starch, or alginic acid; binding agents, for example starch, gelatin or acacia, and lubricating agents, for example magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxyethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the invention may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavouring and colouring agents.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present invention may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug. Such materials are cocoa butter and polyethylene glycols.

For topical use, creams, ointments, jellies, solutions or suspensions, etc., containing the compounds of the present invention are employed. (For purposes of this application, topical application shall include mouthwashes and gargles.)

The pharmaceutical composition and method of the present invention may further comprise other therapeutically active compounds as noted herein which are usually applied in the treatment of the above mentioned pathological conditions.

In the treatment or prevention of conditions which require NK-3 receptor modulation an appropriate dosage level will generally be about 0.01 to 500 mg per kg patient body weight per day which can be administered in single or multiple doses. Preferably, the dosage level will be about 0.1 to about 250 mg/kg per day; more preferably about 0.5 to about 100 mg/kg per day. A suitable dosage level may be about 0.01 to 250 mg/kg per day, about 0.05 to 100 mg/kg per day, or about 0.1 to 50 mg/kg per day. Within this range the dosage may be 0.05 to 0.5, 0.5 to 5 or 5 to 50 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, general health, sex, diet, mode and time of administration, rate of excretion, drug combination, the severity of the particular condition, and the host undergoing therapy.

The present invention also provides pharmaceutical compositions comprising a compound of formula (I) or a pharmaceutically acceptable salt thereof and a pharmaceutically acceptable excipient.

Thus, there is provided a compound of formula (I) or a pharmaceutically acceptable salt thereof for use in a method of treatment of the human or animal body by therapy.

Likewise, there is provided the use of a compound of formula (I) for the manufacture of a medicament for treating a neurokinin-2 and/or neurokinin-3 mediated disease.

There is also disclosed a method of treatment of a subject suffering from a neurokinin-2 and/or neurokinin-3 mediated disease, which comprises administering to that patient a therapeutically effective amount of a compound of formula (I) or a pharmaceutically acceptable salt thereof.

Examples of diseases mediated by neurokinin-2 and/or neurokinin 3 include CNS disorders such as depression (which term includes bipolar (manic) depression (including type I and type II), unipolar depression, single or recurrent major depressive episodes with or without psychotic features, catatonic features, melancholic features, atypical features (e.g. lethargy, over-eating/obesity, hypersomnia) or postpartum onset, seasonal affective disorder and dysthymia, depression-related anxiety, psychotic depression, and depressive disorders resulting from a general medical condition including, but not limited to, myocardial infarction, diabetes, miscarriage or abortion); anxiety disorders (including generalised anxiety disorder (GAD), social anxiety disorder (SAD), agitation, tension, social or emotional withdrawal in psychotic patients, panic disorder, and obsessive compulsive disorder); phobias (including agoraphobia and social phobia); psychosis and psychotic disorders (including schizophrenia, schizo-affective disorder, schizophreniform-diseases, -acute psychosis, alcohol psychosis, autism, delirium, mania (including acute mania), manic depressive psychosis, hallucination, endogenous psychosis, organic psychosyndrome, paranoid and delusional disorders, puerperal psychosis, and psychosis associated with neurodegenerative diseases such as Alzheimer's disease); post-traumatic stress disorder; attention deficit hyperactive disorder (ADHD); cognitive impairment (e.g. the treatment of impairment of cognitive functions including attention, orientation, memory (memory disorders, amnesia, amnesic disorders and age-associated memory impairment) and language function, and including cognitive impairment as a result of stroke, Alzheimer's disease, Aids-related dementia or other dementia states, as well as other acute or sub-acute conditions that may cause cognitive decline such as delirium or depression (pseudodementia states)); convulsive disorders such as epilepsy (which includes simple partial seizures, complex partial seizures, secondary generalised seizures, generalised seizures including absence seizures, myoclonic seizures, clonic seizures, tonic seizures, tonic clonic seizures and atonic seizures); psychosexual dysfunction (including inhibited sexual desire (low libido), inhibited sexual arousal or excitement, orgasm dysfunction, inhibited female orgasm and inhibited male orgasm, hypoactive sexual desire disorder (HSDD), female sexual desire disorder (FSDD), and sexual dysfunction side-effects induced by treatment with antidepressants of the SSRI-class); sleep disorders (including disturbances of circadian rhythm, dyssomnia, insomnia, sleep apnea and narcolepsy); disorders of eating behaviours (including anorexia nervosa and bulimia nervosa); neurodegenerative diseases (such as Alzheimer's disease, ALS, motor neuron disease and other motor disorders such as Parkinson's disease (including relief from locomotor deficits and/or motor disability, including slowly increasing disability in purposeful movement, tremors, bradykinesia, hyperkinesia (moderate and severe), akinesia, rigidity, disturbance of balance and co-ordination, and a disturbance of posture), dementia in Parkinson's disease, dementia in Huntington's disease, neuroleptic-induced Parkinsonism and tardive dyskinesias, neurodegeneration following stroke, cardiac arrest, pulmonary bypass, traumatic brain injury, spinal cord injury or the like, and demyelinating diseases such as multiple sclerosis and amyotrophiclateral sclerosis); withdrawal from abuse of drugs including smoking cessation or reduction in level or frequency of such activities (such as abuse of cocaine, ethanol, nicotine, benzodiazepines, alcohol, caffeine, phencyclidine and phencyclidine-like compounds, opiates such as cannabis, heroin, morphine, sedative, hypnotic, amphetamine or amphetamine-related drugs such as dextroamphetamine, methylamphetamine or a combination thereof); pain (which includes neuropathic pain (including diabetic neuropathy; sciatica; non-specific lower back pain; multiple sclerosis pain; pain associated with fibromyalgia or cancer; AIDS-related and HIV-related neuropathy; chemotherapy-induced neuropathy; neuralgia, such as post-herpetic neuralgia and trigeminal neuralgia; sympathetically maintained pain and pain resulting from physical trauma, amputation, cancer, toxins or chronic inflammatory conditions such as rheumatoid arthritis and osteoarthritis; reflex sympathetic dystrophy such as shoulder/hand syndrome), acute pain (e.g. musculoskeletal pain, post operative pain and surgical pain), inflammatory pain and chronic pain, pain associated with normally non-painful sensations such as “pins and needles” (paraesthesias and dysesthesias), increased sensitivity to touch (hyperesthesia), painful sensation following innocuous stimulation (dynamic, static or thermal allodynia), increased, sensitivity, to noxious stimuli (thermal, cold, mechanical hyperalgesia), continuing pain sensation after removal of the stimulation (hyperpathia) or an absence of or deficit in selective sensory pathways (hypoalgesia), pain associated with migraine, and non-cardiac chest pain); certain CNS-mediated disorders such as emesis, irritable bowel syndrome, and non-ulcer dyspepsia; COPD, asthma, cough, gastro-oesophageal reflex induced cough, and exacerbated asthma; urinary incontinence; hypertension; and conditions associated with platelet hyperaggregability such as tissue ulceration, nephrotic syndrome, diabetes, migraine, coronary artery disease, pre-eclampsia and stroke. Preferably, the compounds of the invention are useful for the treatment of depression; anxiety disorders; phobias; psychosis and psychotic disorders; post-traumatic stress disorder; attention deficit hyperactive disorder (ADHD); withdrawal from abuse of drugs including smoking cessation or reduction in level or frequency of such activities; and irritable bowel syndrome. More preferably, the compounds of the invention are useful for the treatment of depression; anxiety disorders; phobias; and psychosis and psychotic disorders (especially schizophrenia, schizo-affective disorder, and schizophreniform diseases. Most preferably, the compounds of the invention are useful for the treatment of schizophrenia.

The compounds for use in the present invention are generally active in the following tests. They normally have an IC₅₀ of less than 1 μM and preferably less than 100 nM.

Details of the NK-2 receptor and its heterologous expression can be found in Gerard et al., J. Biol. Chem., 265: 20455-20462, 1990 and Huang et al., Biochem., 33: 3007-3013, 1994. The latter paper also contains details of mutant scanning.

Details of the NK-3 receptor and its heterologous expression can be found in Huang et al, BBRC, 1992, 184: 966-972 and Sadowski et al., Neuropeptides, 1993, 24: 317-319.

A membrane preparation is prepared as follows. A 10-layer cell factory is seeded with CHO cells stably expressing NK-3 receptors. The CHO cells are prepared in a triple T175 flask in 11 growth medium which contains Iscore's modified Dulbecco's medium containing 10 ml/1200 mM L-Glutamine, 10 ml/l penicillin-streptomycin, one vial of hypoxanthine-thymidine 500×/l, 1 mg/ml geneticin and 10% fetal bovine serum (inactivated). The cells are grown for 3 days in an incubator. The medium is washed off and the factory is rinsed twice with 400 ml PBS (Ca, Mg-free). 400 ml enzyme free dissoc. solution (EFDS) is added and the factory is maintained for 10 min at room temperature. The cells are dislodged and the suspension poured into 500 ml centrifuge bottles. The process is repeated with 200 ml EFDS and the mixtures pooled giving 6 bottles in all, which are spun in a centrifuge for 10 min at 2200 rpm.

The supernatants are aspirated and the residual cell pellets are frozen at −80° for 30 min to improve cell lysis and then resuspended in 40 ml Tris with inhibitors per cell factory. The cells are homogenized in 40 ml aliquots with 8 strokes of a glass-teflon grinder at setting 40. The homogenate is transferred to 50 ml centrifuge tubes and placed on a rocker for 15 min at r.t. The homogenate is rehomogenised and held on ice if necessary before being centrifuged again as above.

The supernatant is transferred to Sorvall tubes for an SS-34 roter and held on ice.

40 ml cold Tris with inhibitors is used to resuspend and combine the pellets which are again spun as above. The supernatants are again transferred to Sorvall tubes which, with those above, are spun at 18000 rpm for 20 min.

The supernatants are discarded and the pellets resuspended in a Storage Buffer consisting of 2.50 ml 1M Tris pH7.4, 50 μl 1000× protease inhibitors (4 mg/ml leupeptin (Sigmo), 40 mg/ml Bacitracin (Sigma) and 10 mM phosphoranidon (Peninsula) all dissolved in water) plus 0.5 ml 0.5M MnCl₂ made up to 50 ml with H₂O_{dd}. A 10 ml syringe is used with 20-, 23- and 25-gauge needles sequentially.

A Bradford protein assay in conducted on 2-10 μl aliquots with BSA as standard before 500-1000 μl aliquots are snap-frozen in liquid nitrogen for storage at −80° C.

The membrane binding assay is carried out as follows. The amount of membranes needed to specifically bind ≦10% of ¹²⁵I-NeurokinB is predetermined. The frozen stocks are then diluted to allow addition in 50 μl.

The test compounds are dissolved in DMSO. An automated apparatus (Tecan) is programmed to add 5 μl of compound or DMSO, approximately 100,000 cpm of isotope in 20 μl buffer which is prepared from 50 μMTris, pH7.5, 150 ↑M NaCl, bovine serum albumin to 0.02%, and protease inhibitors as in the storage buffer, made up as 0.5M stock, and 175 μl assay buffer (as the storage buffer but containing 5 μM MnCl₂ and without NaCl) into deep well Marsh boxes (Marsh Biomedical Products) in a 96-well format. Excess unlabelled competing peptide is added by hand for non-specific binding as indicated below. The binding reaction is initiated by adding 50 μl of cell membranes. The tubes are incubated with shaking for 1 h at r.t. and filtered on a Tomtec 96 well cell harvester using Mach III filtermats (Tomtec) or using either a Packard 96-well harvester or Tomtec 9600 using Unifilter GF/C (Packard), presoaked in 0.25% polyethyleneimine and washed five times with 1× wash buffer (0.1M·Tris, pH7.4 and 1M NaCl, 1×=100 ml of 10× stock per litre of cold distilled water). If using Unifilter plates, 60 μl Microscint 20 (Packard) is added to each well and the plate is then heat-sealed before counting in a Packard Topcount. Alternatively the filters from the filtermat are placed in 75×100 mm plastic tubes and counted on a Cobra gamma counter.

For the assay, typically 10 μg of membrane is used at 25,000 cpm which is filtered over a Unifilter GF/C presoaked in 0.5% BSA.

Assays for binding at the neurokinin-2 receptor can be carried out in an analogous manner.

Compounds of formula (I) of the type:

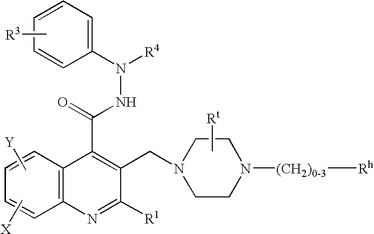

can be made by reacting a compound of formula (II) with a compound of formula (III):

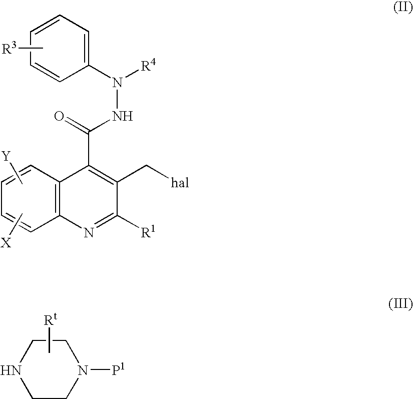

where R¹, R³, R⁴, X and Y are as defined above, hal is a halogen atom (such as chlorine or bromine), R^{t} is hydrogen or oxo, and P¹ is a suitable protecting group, such as tert-butyloxycarbonyl. The reaction may optionally be carried out in the presence of a deprotonating agent, such as LHMDS, in a suitable solvent, such as TUF. The product of the reaction may then be deprotected under suitable conditions and further functionalised by reacting with the compound of formula (IV):

L¹-CH₂)₀₋₃R^{h} (IV)

where R^{h} is as defined above and L¹ is a suitable leaving group. Examples of the compound of formula (IV) include alkyl anhydrides such as acetic anhydride, arylsulfonyl chlorides, such as benzensulfonyl chloride, and dialkylhaloacetamides such as 2-chloro-N,N-dimethylacetamide. Alternatively, the deprotected product of the reaction of compounds (1I) and (III), may be alkylated by treatment with an aldehyde or a ketone, such as tetrahydropyran-4-one, in the presence of a reducing agent, such as sodium triacetoxyborohydride.

Compounds of formula (I) of the type:

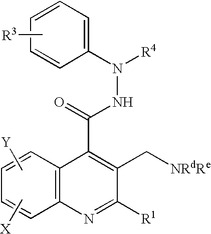

where R¹, R³, R⁴, X, Y, R^{d} and R^{e} are as defined above can be made by reacting a compound of formula (II) with a compound of formula (V):

HNR^{d}R^{e} (V)

where R^{d} and R^{e} are as defined above. The reaction may be carried out at raised temperature or under microwave irradiation in a suitable solvent, such as TUF.

Compounds of formula (I) of the type:

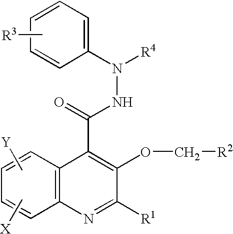

where R¹, R², R³, R⁴, X and Y are as defined above can be prepared by reacting a compound of formula (VI) with a compound of formula (VII):

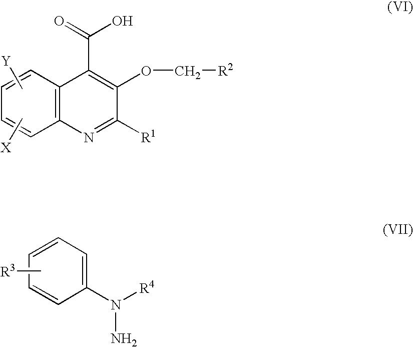

where R¹, R², R³, R⁴, X and Y are as defined above and P² is a suitable protecting group. The reaction may be carried out by pre-reacting the compound of formula (VI) with oxalyl chloride and DMF and then adding the compound of formula (VII). The reaction may be carried out in a suitable solvent, such as dichloromethane.

Alternatively, the compounds of formula (I) of the same type can be prepared by reacting a compound of formula (VIII) with a compound of formula (IX):

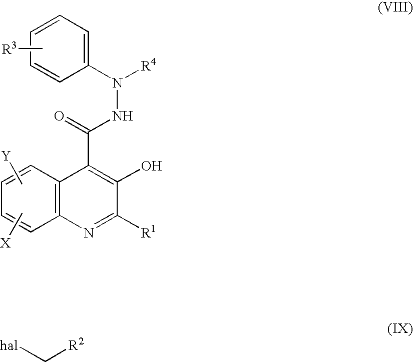

where R¹, R², R³, R⁴, X and Y are as defined above and hal is a halogen atom such as chlorine. The reaction may be carried out in the presence of a base, such as potassium carbonate or sodium iodide, in a suitable solvent, such as TUF.

Compounds of formula (I) can be converted into other compounds of formula (I) using techniques known in the art.

For instance, a compound of formula (I) where R² is C(O)OC₁₋₆alkyl may be converted into a compound of formula (I) where R² is COOH by alkaline or acid hydrolysis under conditions readily apparent to the skilled person.

The compounds of formulae (II) to (IX) are known in the art or can be made by known methods from known compounds.

Where they are not commercially available, the compounds of formulae (II) to (IX) may be prepared by methods analogous to those described in the accompanying Examples, or by standard methods well known from the art.

Where a mixture of products is obtained from any of the processes described above for the preparation of compounds according to the invention, the desired product can be separated therefrom at an appropriate stage by conventional methods such as preparative HPLC or column chromatography utilising, for example, silica and/or alumina in conjunction with an appropriate solvent system.

During any of the above synthetic sequences, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 3^{rd} ed., 1999. The protecting groups may be removed at a convenient subsequent stage using methods known from the art.

¹H nmr spectra were recorded on Bruker AM series spectrometers operating at (reported) frequencies between 300 and 600 MHz. Chemical shifts (6) for signals corresponding to non-exchangeable protons (and exchangeable protons where visible) are recorded in parts per million (ppm) relative to tetramethylsilane and are measured using the residual solvent peak as reference. Signals are reported in the order: number of protons; multiplicity (s, singlet; d, doublet; t, triplet; q, quartet; m, multiplet; br, broad; and combinations thereof); coupling constant(s) in hertz. Mass spectral (MS) data were obtained on a Waters Micromass ZQ or a Waters Micromass ZMD operating in negative (ES⁻) or positive (ES⁺) ionisation mode and results are reported as the ratio of mass over charge (m/z) for the parent ion only. Preparative scale HPLC separations were carried out using mass triggered HPLC on a preparative Agilent 100 separation module. Compounds were either eluted with linear gradients of acetonitrile/0.1% TFA and water/0.1% TFA or with acetonitrile and water (containing ammonium carbonate to give a pH of 10). In all cases flow rates between 15 and 25 mL/min were used.

Abbreviations used herein, particularly the Schemes and Examples, including the following: —

Ac₂O, acetic anhydride; DCE, 1,1-dichloroethene; DMF, N,N-dimethylformamide; DMSO, dimethyl sulfoxide; EtOAc, ethyl acetate; Et₂O, diethyl ether; ES⁺ electrospray; h, hour(s); HPLC, high performance liquid chromatography; LHMDS, lithium hexamethyldisilazide; MeCN, acetonitrile; MeOH, methanol; min, minute(s); NBS, N-bromosuccinimide; PhMe, toluene; RT, room temperature; TFA, trifluoroacetic acid; TUF, tetrahydrofuran.

The following Examples illustrate the present invention:

### Description 1: 3-Methyl-2-phenylquinoline-4-carboxylic acid

To a slurry of isatin (20 g, 0.136 mol) in acetic acid (370 mL) was added propiophenone (18 mL, 0.136 mol) and the mixture was heated in an oil bath at 75° C. for 5 min before adding HCl concentrated (124 mL). Heating was continued at 105° C. for 16 h and the mixture was cooled to RT followed by addition of H₂O (800 mL). The solid which formed was collected by filtration, washed with Et₂O and dried in vacuo at 60° C. to leave 15.6 g (43%) of the title compound as a brown solid. m/z (ES⁺) 264 [M+H⁺].

### Description 2: 3-Methyl-N′,2-diphenylquinoline-4-carbohydrazide

To a suspension of 3-methyl-2-phenylquinoline-4-carboxylic acid (Description 1, 5.0 g, 0.019 mol) in CH₂Cl₂ (60 mL) was added oxalyl chloride (3.32 mL, 0.038 mol) followed by DMF (2 drops). The mixture was stirred at RT for 1 h and the solvent was then evaporated in vacuo. To a solution of the residue in CH₂Cl₂ (30 mL) was added Et₃N (5.30 mL, 0.038 mol) followed by a solution of phenylhydrazine (2.80 mL, 0.028 mol) in CH₂Cl₂ (30 mL). The mixture was stirred at RT for 5 h, diluted with H₂O/brine and the product extracted with CH₂Cl₂ (×5). The combined organic layers were dried (MgSO₄), evaporated in vacuo and the residue was purified by flash chromatography on silica gel eluting with 30% EtOAc/isohexane to leave 4.0 g of the title compound (59%). m/z (ES⁺) 354 [M+H⁺]

### Description 3: Methyl 2-[(3-methyl-2-phenylquinolin-4-yl)carbonyl]-1-phenylhydrazinecarboxylate

To a suspension of 3-methyl-N′,2-diphenylquinoline-4-carbohydrazide (Description 2, 4.0 g, 0.011 mol) in PhMe (120 mL) was added methyl chloroformate (1.75 mL, 0.022 mol) and the mixture was refluxed at 110° C. for 16 h. The mixture was diluted with H₂O and the product extracted with EtOAc (×3). The combined organic phases were dried (MgSO₄) and evaporated in vacuo to give 4.26 g of the title compound (95%). m/z (ES⁺) 412 [M+H⁺]

### Description 4: Dimethyl 1-{[3-(bromomethyl)-2-phenylquinolin-4-yl]carbonyl}-2-phenylhydrazine-1,2-dicarboxylate

A solution of methyl 2-[(3-methyl-2-phenylquinolin-4-yl)carbonyl]-1-phenylhydrazinecarboxylate (Description 3, 4.26 g. 0.010 mol) in THF (100 ml) was cooled at −78° C. and LHMDS 1M in THF (15.6 mL, 0.015 mol) was added followed by methyl chloroformate (1.6 mL, 0.020 mol). The cooling bath was removed and the reaction allowed to warm to +15° C. and then partitioned between H₂O and EtOAc and extracted further with EtOAc. The combined organic layers were dried (MgSO₄) and evaporated to dryness. The residue obtained was dissolved in CCl₄ (100 mL), NBS (3.70 g, 0.020 mol) was added and the mixture was exposed to the light of a quartz lamp for 90 min. The reaction mixture was then left cooling to RT, the solid was filtered off washing with CCl₄ and the filtrate evaporated to dryness. This was dissolved in EtOAc, washed with H₂O, dried (MgSO₄) and evaporated in vacuo. The residue was purified by flash chromatography on silica gel eluting with 20% EtOAc/isohexane to leave 4.12 g of the title compound as a white solid (76%). m/z (ES⁺) 548/550 [M+H⁺].

### Description 5: 3-(Bromomethyl)-2-phenylquinoline-4-carboxylic acid

A suspension of 3-methyl-2-phenylquinoline-4-carboxylic acid (Description 1, 0.200 g, 0.76 mmol) and NBS (0.270 g, 1.52 mmol) in DCE (20 mL) was exposed to the light of a quartz lamp for 5 h. The cooled (RT) reaction mixture was evaporated in vacuo and the residue was dissolved in EtOAc and water and the product was extracted with EtOAc (×2). The organic layers were combined, dried (MgSO₄) and evaporated in vacuo. The residue was purified by flash chromatography on silica gel eluting with EtOAc/isohexane/AcOH (70:30:1) to give the title compound as a mixture with succinimide and the 3-(chloromethyl)-2-phenylquinoline-4-carboxylic acid analogue. m/z (ES⁺) 342/344 [M+H⁺]

### Description 6: Methyl 2-{[3-(chloromethyl)-2-phenylquinolin-4-yl]carbonyl}-1-phenylhydrazinecarboxylate

To a suspension of 3-(bromomethyl)-2-phenylquinoline-4-carboxylic acid (Description 5, 0.100 g, 0.29 mmol) in CH₂Cl₂ (2 mL) was added oxalyl chloride (0.051 mL, 0.58 mmol) followed by DMF (1 drop) and the mixture stirred at RT for 1 h. The solvent was then evaporated in vacuo and the residue dissolved in CH₂Cl₂ (2 mL), Et₃N (0.081 mL, 0.58 mmol) was added followed by methyl 1-phenylhydraziniumcarboxylate hydrochloride (Description 10, 0.060 g, 0.29 mmol) and the mixture stirred at RT overnight. The reaction mixture was partitioned between water and CH₂Cl₂, extracted with CH₂Cl₂ (×2) and the organic layers were dried (MgSO₄) and evaporated in vacuo. The residue was purified by flash chromatography on silica gel eluting with isohexane/EtOAc (70:30) to give 0.043 g of the title compound (33%). m/z (ES⁺) 446/448 [M+H⁺]

### Description 7: Methyl 2-[(3-bromomethyl-2-phenylquinolin-4-yl)carbonyl]-1-phenylhydrazinecarboxylate

A suspension of methyl 2-[(3-methyl-2-phenylquinolin-4-yl)carbonyl]-1-phenylhydrazinecarboxylate (Description 3, 0.15 g, 0.365 mmol) and NBS (0.129 g, 0.725 mmol) in CCl₄ (20 ml) was illuminated by the light of a quartz lamp for 1.5 h allowing the temperature to increase to approx. 75° C. The solvent was removed in vacuo and a solution of the residue dissolved in CH₂Cl₂ was purified on silica gel eluting with 10% to 20% EtOAc in isohexane to give the title compound after evaporation and crystallisation from Et₂O:isohexane (1:1). m/z (ES⁺) 490/492(M+H).

### Description 8: 2-Benzyl 1-methyl 1-phenylhydrazine-1,2-dicarboxylate

To a stirred cooled (5° C.) solution of phenyl hydrazine (20 g, 0.185 mol) in EtOAc and saturated NaHCO₃ (200 mL) was added K₂CO₃ (10 g, 0.72 mol) followed by the slow addition (over 0.5 h) of a solution of benzyl chloroformate (34.8 g, 0.204 mol) in EtOAc (100 mL). The mixture was stirred at 0° C. to 5° C. for 0.5 h and the organic phase was dried (MgSO₄). The solvent was removed in vacuo and the residue was washed with isohexane (100 mL twice) and dried to give 43.9 g of the title compound. ¹H NMR (CDCl₃, 500 Mz) δ 7.42-7.05 (11H, m), 5.23 (2H, m), 3.79 (3H, m) as a mixture of rotamers.

### Description 9: 1-Benzyl 1,2-dimethyl 2-phenylhydrazine 1,1,2-tricarboxylate

To a solution of the product of Description 8 was suspended in PhMe (500 mL) was added methyl chloroformate (17.5 mL, 0.226 mol). The mixture was heated at 100° C. for 1.5 h and the resulting clear solution was cooled to RT and evaporated to dryness to give the title compound as a foam (58 g).

### Description 10: Methyl 1-phenylhydraziniumcarboxylate chloride

A mixture of the product of Description 9 (58 g) and 10% Pd on C (4 g) in MeOH (400 mL) was hydrogenated for 1 h at 30-40 psi of H₂. The mixture was filtered and 1 M HCl in MeOH (200 mL) was added to the filtrate. The solution was evaporated to dryness and the solid residue was washed with Et₂O to give 31.9 g of the title compound. ¹H NMR (CDCl₃, 500 Mz) δ 9.2 (3H, broad s), 7.55 (2H, d, J 7.5), 7.45 (2H, t, J 6.7), 7.35 (1H, t, J 7.5), 3.73 (3H, s) as a mixture of rotamers.

### Description 11: 3-(Benzyloxy)-2-phenylquinoline-4-carboxylic acid

To a suspension of 3-hydroxy-2-phenylquinoline-4-carboxylic acid (2.65 g, 0.01 mol, as described in Giardina et al., J. Med. Chem. 1999, 42, 1053-1065) and K₂CO₃ (5.53 g, 0.04 mol) in THF (50 mL) was added benzyl bromide (2.99 mL, 0.025 mol) and NaI (0.01 g) and the mixture was heated under reflux for 14 h. After this time, the reaction mixture was reduced in volume to ˜20 mL and a further portion of benzyl bromide (1 mL. 0.008 mol) was added and heating was continued under reflux for a further 24 h. The reaction mixture was then filtered, concentrated in vacuo, dissolved in methanol (100 mL) and treated with 2N NaOH solution (25 mL) under reflux temperature for 4 h. The solvents were removed under vacuum and the residue was partitioned between H₂O (100 mL) and Et₂O (2×100 mL). The aqueous layer was acidified with concentrated HCl and the solid produced was collected by filtration and washed first with H₂O then Et₂O in the sinter funnel and then dried in vacuo at 80° C. to leave 2.45 g of the title compound as a white solid. m/z (ES⁺) 356 [M+H⁺]

### Description 12: Methyl 2-[(3-hydroxy-2-phenylquinolin-4-yl)carbonyl]-1-phenylhydrazinecarboxylate

To a solution of methyl 2-{[3-(benzyloxy)-2-phenylquinolin-4-yl]carbonyl}-1-phenylhydrazinecarboxylate (Example 95, 2.4 g, 0.048 mmol) in MeOH (80 mL) under an atmosphere of N₂ was added 10% Pd on C catalyst (0.2 g) then the reaction mixture was shaken on a Parr apparatus under 50 psi of H₂ for 18 h. The reaction mixture was filtered and the filtrate was concentrated in vacuo. The residue was recrystallised from isopropanol to give 0.73 g of the title compound as a white solid. m/z (ES⁺) 414 [M+H⁺].

### Description 13: 4-Pyridylsulfonyl chloride

To a cooled (0° C.) solution of 4-mercaptopyridine (250 mg, 2.25 mmol) in conc. HCl (2 mL) was added 35% H₂O₂ solution (0.25 mL, 2.57 mmol) with stirring. After 30 min, chlorine gas was passed through the solution for 1.5 h then ice was added and the volume adjusted by addition of water to 10 mL. A portion of this crude mixture was used immediately for the preparation of Example 12.

### EXAMPLE 1

### tert-Butyl-4-[(4-{[2-(methoxycarbonyl)-2-phenylhydrazino]carbonyl}-2-phenylquinolin-3-yl)methyl]piperazine-1-carboxylate

### Method F

To a solution of dimethyl 1-{[3-(bromomethyl)-2-phenylquinolin-4-yl]carbonyl}-2-phenylhydrazine-1,2-dicarboxylate (Description 4, 1.5 g, 2.74 mmol) in Et₂O (20 mL) was added a solution of N-butoxycarbonylpiperazine (1.02 g, 5.48 mmol) in Et₂O (10 mL). The suspension was stirred at RT for 16 h when CH₂Cl₂ (30 mL) was added followed by pyrrolidine (2.5 mL, 41.3 mmol). The clear solution was stirred at RT for 11 h then the solvent was removed in vacuo. The residue was partioned between CH₂Cl₂ and saturated NaHCO₃ and the organic phase was dried (MgSO₄) and evaporated to a small volume. The solution was applied to a column containing silica gel and the product eluted with increasing concentrations (10% to 50%) EtOAc in isohexane to give the title compound as a foam (1.54 g) after evaporation. m/z (ES⁺) 596 [M+H⁺].

### EXAMPLE 2

### Methyl 1-phenyl-2-{[2-phenyl-3-(piperazin-1-ylmethyl)quinolin-4-yl]carbonyl}hydrazinecarboxylate

tert-Butyl-4-[(4-{[2-(methoxycarbonyl)-2-phenylhydrazino]carbonyl}-2-phenylquinolin-3-yl)methyl]piperazine-1-carboxylate (Example 1, 0.842 g, 1.41 mmol) was dissolved in anhydrous TFA (20 mL). After 1 h, the solution was evaporated to dryness and the residue dissolved in EtOAc (50 mL) and aqueous K₂CO₃ (50 mL). The organic phase was washed with saturated brine, dried (MgSO₄) and evaporated to dryness. Upon addition of Et₂O, a colourless solid formed. The suspension was evaporated to dryness to give the title compound. ¹H NMR (500 MHz, DMSO d6) δ 11.5 (1H, s), 8.3 (2H, broad s), 8.07 (1H, d, J 3.4), 7.84 (1H, t), 7.7 (1H, broad s), 7.54-7.46 (9H, m), 7.34 (1H, t), 3.82 (3H, s), 3.7-3.5 (2H, v. broad m), 2.6 (4H, broad s), 2.1 (4H, broad s). m/z (ES⁺) 496 [M+H⁺].

### EXAMPLE 3

### Methyl 2-[(3-{[4-(phenylsulfonyl)piperazin-1-yl]methyl}-2-phenylquinolin-4-yl)carbonyl]-1-phenylhydrazine carboxylate

### Method A

Methyl 1-phenyl-2-{[2-phenyl-3-(piperazin-1-ylmethyl)quinolin-4-yl]carbonyl}hydrazinecarboxylate (Example 2, 15 mg, 0.030 mmol) was dissolved in CH₂Cl₂ (0.5 mL) and to this was added Et₃N (0.011 mL, 0.079 mmol) and benzenesulfonyl chloride (16 mg, 0.091 mmol). The solution was stirred at RT for 20 min, evaporated to dryness and a solution of the residue in DMSO (0.5 ml) was purified by preparative HPLC. The fractions from the column which contained product were applied to a Bond Elut™ cartridge (SCX, 0.5 g), washed with MeOH and the product eluted with 2 M NH₃ in MeOH (5 mL). The solution was evaporated to dryness to give 5.2 mg of the title compound. m/z (ES⁺) 637 (M+H).

### EXAMPLE 4

### Methyl 2-({3-[(4-acetylpiperazin-1-yl)methyl]-2-phenylquinolin-4-yl}carbonyl)-1-phenylhydrazinecarboxylate

### Method B

Methyl 1-phenyl-2-{[2-phenyl-3-(piperazin-1-ylmethyl)quinolin-4-yl]carbonyl}hydrazinecarboxylate (Example 2, 16.4 mg, 0.033 mmol) was dissolved in CH₂Cl₂ (0.5 mL) and to this was added Ac₂O (0.014 mL, 0.148 mmol). The solution was stirred at RT for 1 h and then evaporated to dryness. The residue dissolved in MeOH was applied to a Bond Elut™ cartridge (SCX, 0.5 g), washed with MeOH and the product eluted with 2 M NH₃ in MeOH (5 mL). The solution was evaporated to dryness to give 14.7 mg of the title compound. m/z (ES⁺) 538 (M+H).

The following compounds were prepared by a method analogous to that described in Example 3 (Method A) or that described in Example 4 (Method B).

| | | | MS | | |
|---|---|---|---|---|---|
| Ex. | reagent | R | (EI⁺) | method | Chemical name |
| 4 | acetic | COMe | 538 | B | Methyl 2-({3-[(4-acetylpiperazin-1- |
| | anhydride | | | | yl)methyl]-2-phenylquinolin-4-yl}carbonyl)- |
| | | | | | 1-phenylhydrazinecarboxylate |
| 5 | propionic | COEt | 552 | B | methyl 1-phenyl-2-({2-phenyl-3-[(4- |
| | anhydride | | | | propionylpiperazin-1-yl)methyl]quinolin-4- |
| | | | | | yl}carbonyl)hydrazinecarboxylate |
| 6 | isobutyric | COⁱPr | 566 | B | methyl 2-({3-[(4-isobutyrylpiperazin-1- |
| | anhydride | | | | yl)methyl]-2-phenylquinolin-4-yl}carbonyl)- |
| | | | | | 1-phenylhydrazinecarboxylate |
| 7 | pivaloyl | CO^{t}Bu | 581 | A | methyl 2-[(3-{[4-(2,2-dimethylpropanoyl) |
| | chloride | | | | piperazin-l-yl]methyl}-2-phenylquinolin-4- |
| | | | | | yl)carbonyl]-1-phenylhydrazinecarboxylate |
| 8 | benzoyl | COPh | 600 | B | methyl 2-({3-[(4-benzoylpiperazin-1- |
| | chloride | | | | yl)methyl]-2-phenylquinolin-4-yl}carbonyl)- |
| | | | | | 1-phenylhydrazinecarboxylate |
| 9 | phenyl- | COCH₂Ph | 614 | B | methyl 1-phenyl-2-[(2-phenyl-3-{[4- |
| | acetyl | | | | (phenylacetyl)piperazin-1-yl]methyl} |
| | chloride | | | | quinolin-4-yl)carbonyl]hydrazinecarboxylate |
| 10 | methyl | CO₂Me | 554 | B | methyl 4-[(4-{[2-(methoxycarbonyl)-2- |
| | chloro- | | | | phenylhydrazino]carbonyl}-2- |
| | formate | | | | phenylquinolin-3-yl)methyl]piperazine-1- |
| | | | | | carboxylate |
| 11 | isobutyric | CO₂CH₂ⁱPr | 596 | B | isobutyryl 4-[(4-{[2-(methoxycarbonyl)-2- |
| | anhydride | | | | phenylhydrazino]carbonyl}-2- |
| | | | | | phenylquinolin-3-yl)methyl]piperazine-1- |
| | | | | | carboxylate |
| 12 | 4-pyridyl-sulfonylchloride | 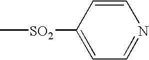 | 637 | A | methyl 1-phenyl-2-[(2-phenyl-3-{[4-(pyridin-4-ylsulfonyl)piperazin-1-yl]methyl}quinolin-4-yl)carbonyl]hydrazinecarboxylate |
| 13 | benzyl- | CO₂CH₂Ph | 630 | B | benzyl 4-[(4-{[2-(methoxycarbonyl)-2- |
| | chloro- | | | | phenylhydrazino]carbonyl}-2- |
| | formate | | | | phenylquinolin-3-yl)methyl]piperazine-1- |
| | | | | | carboxylate |
| 17 | methane- | SO₂Me | 574 | A | methyl 2-[(3-{[4-(methylsulfonyl)piperazin- |
| | sulfonyl | | | | 1-yl]methyl}-2-phenylquinolin-4- |
| | chloride | | | | yl)carbonyl]-1-phenylhydrazinecarboxylate |
| 14 | ethane- | SO₂Et | 588 | A | methyl 2-[(3-{[4-(ethylsulfonyl)piperazin-1- |
| | sulfonyl | | | | yl]methyl}-2-phenylquinolin-4-yl)carbonyl]- |
| | chloride | | | | 1-phenylhydrazinecarboxylate |
| 15 | propane- | SO₂ⁿPr | 602 | A | methyl 1-phenyl-2-[(2-phenyl-3-{[4- |
| | sulfonyl | | | | (propylsulfonyl)piperazin-1-yl]methyl} |
| | chloride | | | | quinolin-4-yl)carbonyl]hydrazinecarboxylate |
| 16 | triflic | SO₂CF₃ | 629 | A | methyl 1-phenyl-2-{[2-phenyl-3-({4- |
| | anhydride | | | | [(trifluoromethyl)sulfonyl]piperazin-1- |
| | | | | | yl}methyl)quinolin-4- |
| | | | | | yl]carbonyl}hydrazinecarboxylat |
| 3 | benzene | SO₂Ph | 636 | A | methyl 2-[(3-{[4-(phenylsulfonyl)piperazin- |
| | sulfonyl | | | | 1-yl]methyl}-2-phenylquinolin-4- |
| | chloride | | | | yl)carbonyl]-1-phenylhydrazinecarboxylate |
| 18 | benzyl- | SO₂CH₂Ph | 651 | A | methyl 2-[(3-{[4-(benzylsulfonyl)piperazin- |
| | sulfonyl | | | | 1-yl]methyl}-2-phenylquinolin-4- |
| | chloride | | | | yl)carbonyl]-1-phenylhydrazinecarboxylate |
| 19 | 2-thienyl-sulfonylchloride | 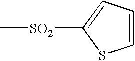 | 642 | A | methyl 1-phenyl-2-[(2-phenyl-3-{[4-(2-thienylsulfonyl)piperazin-1-yl]methyl}quinolin-4-yl)carbonyl]hydrazinecarboxylate |
| 20 | 6-chloro-pyridyl-3-sulfonylchloride | 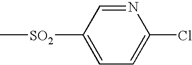 | 671/673 | A | methyl 2-{[3-({4-[(6-chloropyridin-3-yl)sulfonyl]piperazin-1-yl}methyl)-2-phenylquinolin-4-yl]carbonyl}-1-phenylhydrazinecarboxylate |
| 21 | pyridine-3-sulfonylchloride | 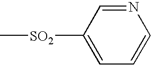 | 637 | A | methyl 1-phenyl-2-[(2-phenyl-3-{[4-(pyridin-3-ylsulfonyl)piperazin-1-yl]methyl}quinolin-4-yl)carbonyl]hydrazinecarboxylate |
| 22 | pyridine-2-sulfonylchloride | 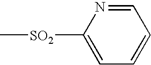 | 637 | A | methyl 1-phenyl-2-[(2-phenyl-3-{[4-(pyridin-2-ylsulfonyl)piperazin-1-yl]methyl}quinolin-4-yl)carbonyl]hydrazinecarboxylate |
| 23 | 1-methyl-4-sulfonylchloride | 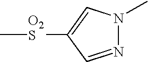 | 640 | A | methyl 2-{[3-({4-[(1-methyl-1-pyrazol-4-yl)sulfonyl]piperazin-1-yl}methyl)-2-phenylquinolin-4-yl]carbonyl}-1-phenylhydrazinecarboxylate |

### EXAMPLE 24

### Methyl 2-[(3-{[4-(tetrahydropyran-4-yl)piperazin-1-yl]methyl}-2-phenylquinolin-4-yl)carbonyl]-1-phenylhydrazine carboxylate

### Method C

To a solution of tetrahydropyran-4-one (0.1 mL, 1.08 mmol) and methyl 1-phenyl-2-{[2-phenyl-3-(piperazin-1-ylmethyl)quinolin-4-yl]carbonyl}hydrazinecarboxylate (Example 2, 134 mg, 0.27 mmol) in CH₂Cl₂ (0.5 mL) was added acetic acid (0.016 mL) and sodium triacetoxyborohydride (115 mg, 0.54 mmol). The solution was stirred at RT for 3 h when 1 M aqueous HCl (0.5 mL) was added followed 15 minutes later by EtOAc (30 mL), H₂O (30 mL) and sufficient NaHCO₃ to adjust the pH to >6. The organic phase was dried (MgSO₄) and evaporated to dryness. The residue was dissolved in the minimum CH₂Cl₂ and this solution was applied to a column containing silica gel and the product eluted by increasing concentrations of MeOH (0-5%) in EtOAc. The fractions containing product were evaporated to dryness and the residue was dissolved in 1:1 CH₂Cl₂: Et₂O to which was subsequently added 1 M HCl in Et₂O (0.24 mL). The suspension was evaporated to give the title compound as a fine colourless solid. ¹H NMR (360 MHz, DMSO d₆, 329° K) δ 11.4 (1H, s), 10.43 (1H, broad s), 8.07 (1H, d J 6.4), 8.0 (1H, broad s), 7.83 (1H, apparent t, J 6.2), 7.76 (1H, apparent t, J 6.2), 7.56-7.45 (8H, m), 7.33 (1H, apparent t, J 5.5), 5.7-4.7 (very broad signal), 3.9 (2H, dd, J 9, 3.5), 3.84 (3H, s), 3.69 (2H, broad s), 3.26 (2H, t, J 8.7), 3.18 (2H, broad s), 2.44-2.29 (6H, m), 1.85 (2H, dm, J 7.6), 1.60 (2H, m). m/z (ES⁺) 580 (M+H).

The following compounds were prepared in an analogous manner and were isolated by a combination of either flash chromatography on silica gel or preparative hplc and Bond Elut™ LR (SCX)

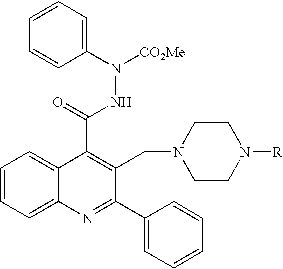

| | | | MS m/z | |
|---|---|---|---|---|
| Ex. | ketone | N—R | (M + H) | Chemical name |
| 25 | cyclopentanone | 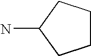 | 564 | methyl 2-({3-[(4-cyclopentylpiperazin-1-yl)methyl]-2-phenylquinolin-4-yl}carbonyl)-1-phenylhydrazinecarboxylate |
| 26 | 3-methylcyclo-hexane | 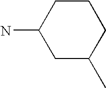 | 593 | methyl 2-[(3-{[4-(3-methylcyclohexyl)piperazin-1-yl]methyl}-2-phenylquinolin-4-yl)carbonyl]-1-phenylhydrazinecarboxylate |
| 27 | 1,4-cyclohexane-dione monoethylene ketal | 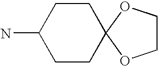 | 636 | methyl 2-[(3-{[4-(1,4-dioxaspiro[4.5]dec-8-yl)piperazin-1-yl]methyl}-2-phenylquinolin-4-yl)carbonyl]-1-phenylhydrazinecarboxylate |

### EXAMPLE 28

### Methyl 2-{[3-({4-[2-(dimethylamino)-2-oxoethyl]piperazin-1-yl}methyl)-2-phenylquinolin-4-yl]carbonyl}-1-phenylhydrazinecarboxylate hydrochloride salt

### Method D

A solution of methyl 1-phenyl-2-{[2-phenyl-3-(piperazin-1-ylmethyl)quinolin-4-yl]carbonyl}hydrazinecarboxylate (Example 2, 138 mg, 0.28 mmol) and Et₃N (0.1 mL) in CH₂Cl₂ (5 mL) was added 2-chloro-N,N-dimethylacetamide (0.086 mL, 0.84 mmol) and the solution was heated for a total of 8 h. The mixture was diluted with CH₂Cl₂ and washed with NaHCO₃ solution, dried (MgSO₄) and evaporated to dryness. A concentrated solution of the residue in CH₂Cl₂ was purified by flash chromatography on silica gel eluting with increasing concentrations of EtOAc in isohexane (20% to 100%). Fractions containing the product were evaporated to a small volume and 2 M HCl in Et₂O (0.1 mL). The product was precipitated by addition of further Et₂O (10 mL) and the suspension evaporated to give the title compound as a solid. ¹H NMR (360 MHz, DMSO d₆ at 329° C.) δ 11.49 (1H, s), 9.46 (1H, broad s), 8.05 (1H, d, J 8.4), 8.00 (1H, broad s), 7.88 (1H, t, J 7.4), 7.66 (1H, t, J 7.7), 7.6-7.45 (8H, m), 7.33 (1H, t, J 7.4), 4.08 (2H, s), 3.83 (3H, s), 3.66 (2H, broad s), 3.14 (2H, broad s), 2.90 (3H, s), 2.86 (3H, s), 2.61 (2H, broad s), 2.4-2.16 (4H, broad m). m/z (ES⁺) 581 (M+H).

The Examples in the following table were prepared using the product of Example 2 as starting material and the appropriate alkylating agent by a method analogous to Method D using either heating in an oil bath or in a microwave reactor and the product was isolated by chromatography on silica gel or by preparative HPLC.

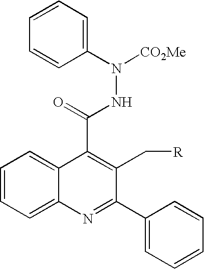

| | alkylation | | MS m/z | | |
|---|---|---|---|---|---|
| Ex. | agent | R | (M + H) | method | Chemical name |
| 29 | 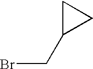 | 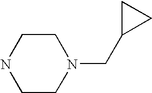 | 550 | D | methyl 2-[(3-{[4-(cyclopropylmethyl)piperazin-1-yl]methyl}-2-phenylquinolin-4-yl)carbonyl]-1-phenylhydrazinecarboxylate |

### Method E

To a solution of methyl 2-{[3-(chloromethyl)-2-phenylquinolin-4-yl]carbonyl}-1-phenylhydrazinecarboxylate (Description 6) in THF was added the amine (2 eq) and the reaction was refluxed at 65° C. overnight (Et₃N was added where the salt of the amine was used). Alternatively the reaction was carried out under microwave irradiation at 120-140° C. for 15-50 min. The solvent was evaporated in vacuo and the residue was purified either by LC-MS or by flash chromatography.

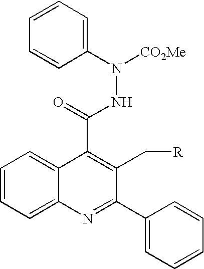

| | | MS m/z | | |
|---|---|---|---|---|
| Ex. | R | (M + H) | method | Chemical name |
| 30 | 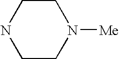 | 510 | E | methyl 2-({3-[(4-methylpiperazin-1-yl)methyl]-2-phenylquinolin-4-yl}carbonyl)-1-phenylhydrazinecarboxylate |
| 31 | 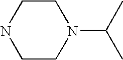 | 538 | E | methyl 2-({3-[(4-isopropylpiperazin-1-yl)methyl]-2-phenylquinolin-4-yl}carbonyl)-1-phenylhydrazinecarboxylate |
| 32 | 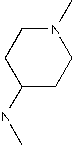 | 538 | F | methyl 2-[(3-{[methyl(1-methylpiperidin-4-yl)amino]methyl}-2-phenylquinolin-4-yl)carbonyl]-1-phenylhydrazinecarboxylate |
| 33 | 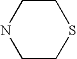 | 513 | F | methyl 1-phenyl-2-{[2-phenyl-3-(thiomorpholin-4-ylmethyl)quinolin-4-yl]carbonyl}hydrazinecarboxylate |
| 34 | 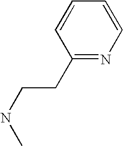 | 546 | E | methyl 2-[(3-{[methyl(2-pyridin-2-ylethyl)amino]methyl}-2-phenylquinolin-4-yl)carbonyl]-1-phenylhydrazinecarboxylate |
| 35 | 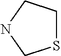 | 499 | E | methyl 1-phenyl-2-{[2-phenyl-3-(1,3-thiazolidin-3-ylmethyl)quinolin-4-yl]carbonyl}hydrazinecarboxylate |
| 36 | 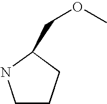 | 525 | E | methyl 2-[(3-{[(2R)-2-(methoxymethyl)pyrrolidin-1-yl]methyl}-2-phenylquinolin-4-yl)carbonyl]-1-phenylhydrazinecarboxylate |
| 37 | 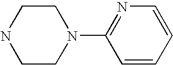 | 573 | E | methyl 1-phenyl-2-({2-phenyl-3-[(4-pyridin-2-ylpiperazin-1-yl)methyl]quinolin-4-yl}carbonyl)hydrazinecarboxylate |
| 38 | 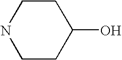 | 511 | E | methyl 2-({3-[(4-hydroxypiperidin-1-yl)methyl]-2-phenylquinolin-4-yl}carbonyl)-1-phenylhydrazinecarboxylate |
| 39 | 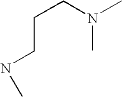 | 526 | E | methyl 2-[(3-{[[3-(dimethylamino)propyl](methyl)amino]methyl}-2-phenylquinolin-4-yl)carbonyl]-1-phenylhydrazinecarboxylate |
| 40 | 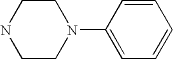 | 572 | E | methyl 1-phenyl-2-({2-phenyl-3-[(4-phenylpiperazin-1-yl)methyl]quinolin-4-yl}carbonyl)hydrazinecarboxylate |
| 41 | 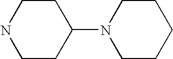 | 578 | E | methyl 2-{[3-(1,4′-bipiperidin-1′-ylmethyl)-2-phenylquinolin-4-yl]carbonyl}-1-phenylhydrazinecarboxylate |
| 42 | 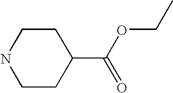 | 567 | E | ethyl 1-[(4-{[2-(methoxycarbonyl)-2-phenylhydrazino]carbonyl}-2-phenylquinolin-3-yl)methyl]piperidine-4-carboxylate |
| 43 | 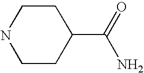 | 538 | E | methyl 2-[(3-{[4-(aminocarbonyl)piperidin-1-yl]methyl}-2-phenylquinolin-4-yl)carbonyl]-1-phenylhydrazinecarboxylate |
| 44 | 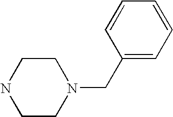 | 586 | E | methyl 2-({3-[(4-benzylpiperazin-1-yl)methyl]-2-phenylquinolin-4-yl}carbonyl)-1-phenylhydrazinecarboxylate |
| 45 | 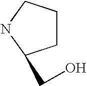 | 511 | E | methyl 2-[(3-{[(2S)-2-(hydroxymethyl)pyrrolidin-1-yl]methyl}-2-phenylquinolin-4-yl)carbonyl]-1-phenylhydrazinecarboxylate |
| 46 | 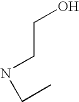 | 499 | E | methyl 2-[(3-{[ethyl(2-hydroxyethyl)amino]methyl}-2-phenylquinolin-4-yl)carbonyl]-1-phenylhydrazinecarboxylate |
| 47 | 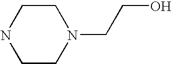 | 540 | E | methyl 2-[(3-{[4-(2-hydroxyethyl)piperazin-1-yl]methyl}-2-phenylquinolin-4-yl)carbonyl]-1-phenylhydrazinecarboxylate |
| 48 | 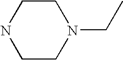 | 524 | E | methyl 2-({3-[(4-ethylpiperazin-1-yl)methyl]-2-phenylquinolin-4-yl}carbonyl)-1-phenylhydrazinecarboxylate |
| 49 | 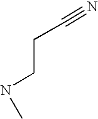 | 494 | E | methyl 2-[(3-{[(2-cyanoethyl)(methyl)amino]methyl}-2-phenylquinolin-4-yl)carbonyl]-1-phenylhydrazinecarboxylate |
| 50 | 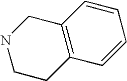 | 543 | E | methyl 2-{[3-(3,4-dihydroisoquinolin-2(1H)-ylmethyl)-2-phenylquinolin-4-yl]carbonyl}-1-phenylhydrazinecarboxylate |
| 51 | 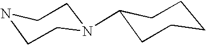 | 578 | E | methyl 2-({3-[(4-cyclohexylpiperazin-1-yl)methyl]-2-phenylquinolin-4-yl}carbonyl)-1-phenylhydrazinecarboxylate |
| 52 | 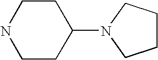 | 564 | E | methyl 1-phenyl-2-({2-phenyl-3-[(4-pyrrolidin-1-ylpiperidin-1-yl)methyl]quinolin-4-yl}carbonyl)hydrazinecarboxylate |
| 53 | 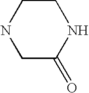 | 510 | E | methyl 2-({3-[(3-oxopiperazin-1-yl)methyl]-2-phenylquinolin-4-yl}carbonyl)-1-phenylhydrazinecarboxylate |
| 54 | 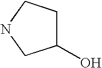 | 497 | E | methyl 2-({3-[(3-hydroxypyrrolidin-1-yl)methyl]-2-phenylquinolin-4-yl}carbonyl)-1-phenylhydrazinecarboxylate |
| 55 | 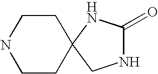 | 565 | E | methyl 2-({3-[(2-oxo-1,3,8-triazaspiro[4.5]dec-8-yl)methyl]-2-phenylquinolin-4-yl}carbonyl)-1-phenylhydrazinecarboxylate |
| 56 | 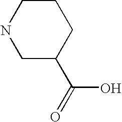 | 539 | E | 1-[(4-{[2-(methoxycarbonyl)-2-phenylhydrazino]carbonyl}-2-phenylquinolin-3-yl)methyl]piperidine-3-carboxylic acid |
| 57 | 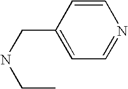 | 546 | E | methyl 2-[(3-{[ethyl(pyridin-4-ylmethyl)amino]methyl}-2-phenylquinolin-4-yl)carbonyl]-1-phenylhydrazinecarboxylate |
| 58 | 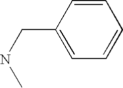 | 531 | E | methyl 2-[(3-{[benzyl(methyl)amino]methyl}-2-phenylquinolin-4-yl)carbonyl]-1-phenylhydrazinecarboxylate |
| 59 | 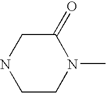 | 524 | E | methyl 2-({3-[(4-methyl-3-oxopiperazin-1-yl)methyl]-2-phenylquinolin-4-yl}carbonyl)-1-phenylhydrazinecarboxylate |
| 60 | 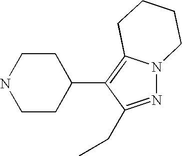 | 643 | E | methyl 2-[(3-{[4-(2-ethyl-4,5,6,7-tetrahydropyrazolo[1,5-a]pyridin-3-yl)piperidin-1-yl]methyl}-2-phenylquinolin-4-yl)carbonyl]-1-phenylhydrazinecarboxylate |
| 61 | 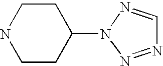 | 563 | E | methyl 1-phenyl-2-[(2-phenyl-3-{[4-(2H-tetrazol-2-yl)piperidin-1-yl]methyl}quinolin-4-yl)carbonyl]hydrazinecarboxylate |
| 62 | 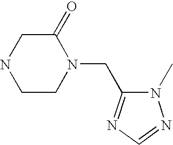 | 605 | E | methyl 2-{[3-({4-[(1-methyl-1H-1,2,4-triazol-5-yl)methyl]-3-oxopiperazin-1-yl}methyl)-2-phenylquinolin-4-yl]carbonyl}-1-phenylhydrazinecarboxylate |
| 63 | 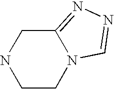 | 534 | E | methyl 2-{[3-(5,6-dihydro[1,2,4]triazolo[4,3-a]pyrazin-7(8H)-ylmethyl)-2-phenylquinolin-4-yl]carbonyl}-1-phenylhydrazinecarboxylate |
| 64 | 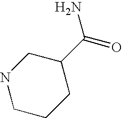 | 538 | E | methyl 2-[(3-{[3-(aminocarbonyl)piperidin-1-yl]methyl}-2-phenylquinolin-4-yl)carbonyl]-1-phenylhydrazinecarboxylate |
| 65 | 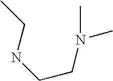 | 526 | E | methyl 2-[(3-{[[2-(dimethylamino)ethyl](ethyl)amino]methyl}-2-phenylquinolin-4-yl)carbonyl]-1-phenylhydrazinecarboxylate |
| 66 | 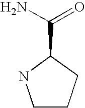 | 524 | E | methyl 2-[(3-{[(2R)-2-(aminocarbonyl)-1-pyrrolidinyl]methyl}-2-phenylquinolin-4-yl)carbonyl]-1-phenylhydrazinecarboxylate |
| 67 | 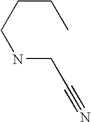 | 522 | E | methyl 2-[(3-{[butyl(cyanomethyl)amino]methyl}-2-phenylquinolin-4-yl)carbonyl]-1-phenylhydrazinecarboxylate |
| 68 | 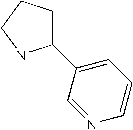 | 558 | E | methyl 1-phenyl-2-({2-phenyl-3-[(2-pyridin-3-ylpyrrolidin-1-yl)methyl]quinolin-4-yl}carbonyl)hydrazinecarboxylate |
| 69 | 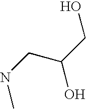 | 515 | E | methyl 2-[(3-{[(2,3-dihydroxypropyl)(methyl)amino]methyl}-2-phenylquinolin-4-yl)carbonyl]-1-phenylhydrazinecarboxylate |
| 70 | 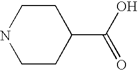 | 539 | E | 1-[(4-{[2-(methoxycarbonyl)-2-phenylhydrazino]carbonyl}-2-phenylquinolin-3-yl)methyl]piperidine-4-carboxylic acid |
| 71 | 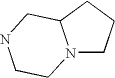 | 536 | E | methyl 2-{[3-(hexahydropyrrolo[1,2-a]pyrazin-2(1H)-ylmethyl)-2-phenylquinolin-4-yl]carbonyl}-1-phenylhydrazinecarboxylate |
| 72 | 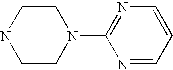 | 574 | E | methyl 1-phenyl-2-({2-phenyl-3-[(4-pyrimidin-2-ylpiperazin-1-yl)methyl]quinolin-4-yl}carbonyl)hydrazinecarboxylate |
| 73 | 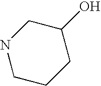 | 511 | E | methyl 2-({3-[(3-hydroxypiperidin-1-yl)methyl]-2-phenylquinolin-4-yl}carbonyl)-1-phenylhydrazinecarboxylate |
| 74 | 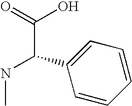 | 575 | E | (2S)-[[(4-{[2-(methoxycarbonyl)-2-phenylhydrazino]carbonyl}-2-phenylquinolin-3-yl)methyl](methyl)amino](phenyl)acetic acid |
| 75 | 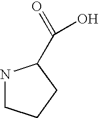 | 525 | E | 1-[(4-{[2-(methoxycarbonyl)-2-phenylhydrazino]carbonyl}-2-phenylquinolin-3-yl)methyl]proline |
| 76 | 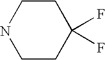 | 531 | E | methyl 2-({3-[(4,4-difluoropiperidin-1-yl)methyl]-2-phenylquinolin-4-yl}carbonyl)-1-phenylhydrazinecarboxylate |
| 77 | 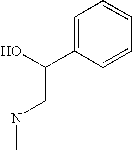 | 561 | E | methyl 2-[(3-{[(2-hydroxy-2-phenylethyl)(methyl)amino]methyl}-2-phenylquinolin-4-yl)carbonyl]-1-phenylhydrazinecarboxylate |
| 78 | 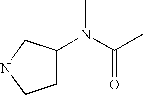 | 552 | E | methyl 2-{[3-({3-[acetyl(methyl)amino]pyrrolidin-1-yl}methyl)-2-phenylquinolin-4-yl]carbonyl}-1-phenylhydrazinecarboxylate |
| 79 | 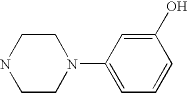 | 588 | E | methyl 2-[(3-{[4-(3-hydroxyphenyl)piperazin-1-yl]methyl}-2-phenylquinolin-4-yl)carbonyl]-1-phenylhydrazinecarboxylate |
| 80 | 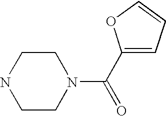 | 590 | E | methyl 2-[(3-{[4-(2-furoyl)piperazin-1-yl]methyl}-2-phenylquinolin-4-yl)carbonyl]-1-phenylhydrazinecarboxylate |
| 81 | 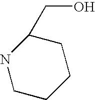 | 525 | E | methyl 2-[(3-{[2-(hydroxymethyl)piperidin-1-yl]methyl}-2-phenylquinolin-4-yl)carbonyl]-1-phenylhydrazinecarboxylate |
| 82 | 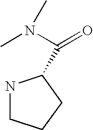 | 552 | E | methyl 2-[(3-{[(2S)-2-[(dimethylamino)carbonyl]-1-pyrrolidinyl]methyl}-2-phenylquinolin-4-yl)carbonyl]-1-phenylhydrazinecarboxylate |
| 83 | 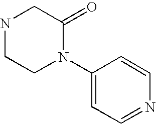 | 587 | E | methyl 2-({3-[(3-oxo-4-pyridin-4-ylpiperazin-1-yl)methyl]-2-phenylquinolin-4-yl}carbonyl)-1-phenylhydrazinecarboxylate |
| 84 | 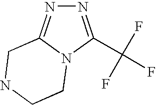 | 602 | E | methyl 1-phenyl-2-[(2-phenyl-3-{[3-(trifluoromethyl)-5,6-dihydro[1,2,4]triazolo[4,3-a]pyrazin-7(8H)-yl]methyl}quinolin-4-yl)carbonyl]hydrazinecarboxylate |
| 85 | 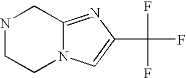 | 601 | E | methyl 1-phenyl-2-[(2-phenyl-3-{[2-(trifluoromethyl)-5,6-dihydroimidazo[1,2-a]pyrazin-7(8H)-yl]methyl}quinolin-4-yl)carbonyl]hydrazinecarboxylate |
| 86 | 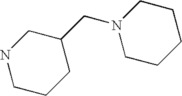 | 592 | E | methyl 1-phenyl-2-[(2-phenyl-3-{[3-(piperidin-1-ylmethyl)piperidin-1-yl]methyl}quinolin-4-yl)carbonyl]hydrazinecarboxylate |
| 87 | 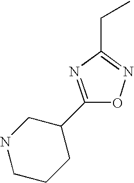 | 591 | E | methyl 2-[(3-{[3-(3-ethyl-1,2,4-oxadiazol-5-yl)piperidin-1-yl]methyl}-2-phenylquinolin-4-yl)carbonyl]-1-phenylhydrazinecarboxylate |
| 88 | 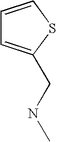 | 537 | E | methyl 2-[(3-{[methyl(2-thienylmethyl)amino]methyl}-2-phenylquinolin-4-yl)carbonyl]-1-phenylhydrazinecarboxylate |
| 89 | 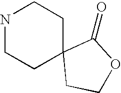 | 565 | E | methyl 2-({3-[(1-oxo-2-oxa-8-azaspiro[4.5]dec-8-yl)methyl]-2-phenylquinolin-4-yl}carbonyl)-1-phenylhydrazinecarboxylate |
| 90 | 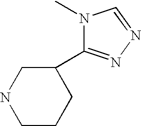 | 576 | E | methyl 2-[(3-{[3-(4-methyl-4H-1,2,4-triazol-3-yl)piperidin-1-yl]methyl}-2-phenylquinolin-4-yl)carbonyl]-1-phenylhydrazinecarboxylate |
| 91 | 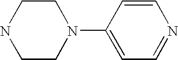 | 573 | E | methyl 1-phenyl-2-({2-phenyl-3-[(4-pyridin-4-ylpiperazin-1-yl)methyl]quinolin-4-yl}carbonyl)hydrazinecarboxylate |
| 92 | 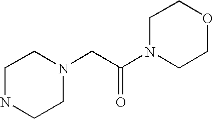 | 623 | E | methyl 2-[(3-{[4-(2-morpholin-4-yl-2-oxoethyl)piperazin-1-yl]methyl}-2-phenylquinolin-4-yl)carbonyl]-1-phenylhydrazinecarboxylate |
| 93 | 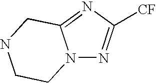 | 602 | E | methyl 1-phenyl-2-[(2-phenyl-3-{[2-(trifluoromethyl)-5,6-dihydro[1,2,4]triazolo[1,5-a]pyrazin-7(8H)-yl]methyl}quinolin-4-yl)carbonyl]hydrazinecarboxylate |

### EXAMPLE 94

### tert-Butyl-4-[(4-{[2-(methoxycarbonyl)-2-phenylhydrazino]carbonyl}-2-phenylquinolin-3-yl)methyl]-2-oxopiperazin-1-carboxylate

To a solution of 4-tert-butoxycarbonylpiperazin-2-one (125 mg, 0.80 mmol) in THF (4 mL) at −78° C. was added 1 M LHMDS in hexane (0.65 mL, 0.65 mmol). To the resultant clear solution was added a solution of dimethyl 1-{[3-(bromomethyl)-2-phenylquinolin-4-yl]carbonyl}-2-phenylhydrazine-1,2-dicarboxylate (Description 4, 336 mg, 0.61 mmol) in THF (4 mL). The solution was allowed to warm to RT overnight. A solution of 4-tert-butoxycarbonylpiperazin-2-one (125 mg, 0.80 mmol) in THF (4 mL) at −78° C. was added 1 M LHMDS in hexane (0.65 mL, 0.65 mmol) and this solution was added to the reaction mixture at RT and the solution was stirred at RT for an additional 16 h. Acetic acid (0.1 mL) was added and the product was partitioned between EtOAc and saturate brine. The organic phase was dried (MgSO₄), evaporated to dryness and the residue was purified by chromatography initially on silica gel (eluting with increasing concentrations of EtOAc in isohexane (0-100%) and subsequently by RP preparative HPLC to give the title compound as a foam (57 mg). ¹H NMR (500 MHz, DMSO d₆) δ 11.7 (1H, s), 8.11 (1H, d, J 8.4), 7.88 (1H, t, J 7.5), 7.72 (1H, broad s), 7.53-7.43 (10H, m), 7.34 (1H, t, J 7.1), 4.93 (2H, broad s), 3.80 (3H, s), 3.41 (2H, s), 3.21 (2H, broad m), 2.79 (1H, m), 2.7 (1H, m). m/z (EI⁺) 610 (M+H).

### EXAMPLE 95

### Methyl 1-phenyl-2-{[2-phenyl-3-(2-oxo-piperazin-1-ylmethyl)quinolin-4-yl]carbonyl}hydrazinecarboxylate

tert-Butyl-4-[(4-{[2-(methoxycarbonyl)-2-phenylhydrazino]carbonyl}-2-phenylquinolin-3-yl)methyl]-2-oxopiperazin-1-carboxylate (Example 94, 47 mg, 0.077 mmol) was dissolved in TFA (5 mL) and the solution stirred for 0.3 h. The solvent was removed by evaporation and the residue was dissolved in MeOH (5 mL). The solution was applied to a Bond Elut™ SCX (0.5 g) washed with MeOH (5 mL) and the product eluted with 2M NH₃ in MeOH (5 mL). The solution was evaporated to give the title compound. ¹H NMR (500 MHz, CDCl₃) δ 11.7 (1H, s), 8.1 (1H, d, J 8.6), 7.86 (1H, t, J 7.5), 7.71 (1H, broad s), 7.56-7.42 (10H, m), 7.32 (1H, t, J 7.0), 4.95 (1H, broad s), 4.5 (1H, broad s), 3.8 (3H, s), 2.75 (3H, m), 2.6 (1H, m). m/z (EI⁺) 510 (M+H).

### EXAMPLE 96

### Methyl 2-{[3-(benzyloxy)-2-phenylquinolin-4-yl]carbonyl}-1-phenylhydrazinecarboxylate

To a solution of 3-benzyloxy-2-phenylquinoline-4-carboxylic acid (Description 11, 2.12 g, 6 mmol) in CH₂Cl₂ (60 mL) was added oxalyl chloride (0.782 mL, 9 mmol) and 1 drop of DMF. The solution was stirred at RT for 1 h then concentrated in vacuo and azeotroped with two further 50 mL portions of CH₂Cl₂. The residue was redissolved in CH₂Cl₂ (50 mL) and added to a solution containing methyl 1-phenylhydraziniumcarboxylate chloride (Description 9, 1.8 g, 9 mmol) and Et₃N (3.3 mL, 24 mmol) in CH₂Cl₂ (90 mL). The clear solution was stirred at RT for 14 h and then was partitioned between CH₂Cl₂ and saturated NaHCO₃ and the organic phase was dried (Na₂SO₄) and evaporated to a small volume. The solution was applied to a column containing silica gel and the product eluted with increasing concentrations (0% to 30%) EtOAc in isohexane to give the title compound as a foam (2.45 g) after evaporation. ¹H NMR (500 MHz, CDCl₃): δ 8.26 (2H, s), 8.17 (1H, d, J 8.3), 7.99 (2H, s), 7.72 (1H, t, J 7.1), 7.63 (1H, t, J 7.2), 7.49 (5H, s), 7.19 (2H, t, J 7.3), 6.92 (2H, d, J 7.2), 4.66 (2H, s), 3.88 (3H, s). m/z (ES⁺) 504 [M+H⁺].

### EXAMPLE 97

### Methyl 2-({3-[(1-methyl-1H-1,2,4-triazol-5-yl)methoxy]-2-phenylquinolin-4-yl}carbonyl)-1-phenylhydrazinecarboxylate

To a solution of methyl 1-phenyl-2-{[2-phenyl-3-(hydroxy)quinolin-4-yl]carbonyl}hydrazinecarboxylate (Description 12, 0.2 g, 0.48 mmol) in THF (5 mL) was added K₂CO₃ (0.2 g, 1.44 mmol) and 2-chloromethyl-1-methyl-triazole hydrochloride (0.088 g, 0.6 mmol) and NaI (5 mg). The solution was heated at 60° C. for 14 h then filtered through a sinter funnel and the filtrate was concentrated in vacuo. The residue was applied to a column containing silica gel and the product was eluted with increasing concentrations (0% to 50%) EtOAc in isohexane to give the title compound as a white solid (70 mg) after evaporation. ¹H NMR (500 MHz, CDCl₃): δ 10.60 (1H, br s), 8.07 (2H, t, J 9.3), 7.88 (2H, d, J 6.5), 7.68 (1H, t, J 7.4), 7.59 (4H, d, J 8.5), 7.52 (3H, d, J 7.2), 7.40 (2H, t, J 6.9), 7.30 (1H, d, J 6.9), 5.00 (2H, s), 3.89 (3H, s), 3.25 (3H, s). m/z (ES⁺) 509 [M+H⁺].

### EXAMPLE 98

### Methyl 2-{[3-(cyanomethoxy)-2-phenylquinolin-4-yl]carbonyl}-1-phenylhydrazinecarboxylate

This compound was prepared according to the procedure outlined in Example 97 replacing 2-chloromethyl-1-methyl-triazole hydrochloride with bromoacetonitrile. ¹H NMR (500 MHz, CDCl₃): 8.35 (1H, s), 8.19 (1H, d, J 8.4), 8.15 (H, br s), 7.99 (2H, d, J 6.3), 7.77 (1H, t, J 7.7), 7.65 (1H, t, J 7.6), 7.60 (2H, d, J 7.3), 7.55 (3H, q, J 6.9), 7.47 (2H, t, J 7.9), 7.35 (1H, t, J 7.4), 4.45 (2H, s), 3.93 (3H, s). m/z (ES⁺) 453 [M+H⁺].

### EXAMPLE 99

### Methyl-2-{[3-(2-methoxy-2-oxoethoxy)-2-phenylquinolin-4-yl]carbonyl}-1-phenylhydrazinecarboxylate

This compound was prepared according to the procedure outlined in Example 97 replacing 2-chloromethyl-1-methyl-triazole hydrochloride with methylbromoacetate. ¹H NMR (500 MHz, CDCl₃): 9.39 (1H, s), 8.13 (2H, d, J 8.1), 7.97 (2H, d, J 6.8), 7.69 (1H, d, J 7.7), 7.59 (2H, s), 7.50 (3H, d, J 7.5), 7.40 (2H, t, J 7.4), 7.29 (2H, s), 4.30 (2H, s), 3.88 (3H, s), 3.58 (3H, s). m/z (ES⁺) 486 [M+H⁺].

### EXAMPLE 100

### [(4-{[2-(Methoxycarbonyl)-2-phenylhydrazino]carbonyl}-2-phenylquinolin-3-yl)oxy]acetic acid

To a solution of methyl-2-{[3-(2-methoxy-2-oxoethoxy)-2-phenylquinolin-4-yl]carbonyl}-1-phenylhydrazinecarboxylate (Example 99, 0.248 g, 0.00051 mol) in methanol (10 mL) was added 2 N NaOH solution (2 mL) and the mixture was stirred at RT for 16 h. Water (20 mL) was added and the aqueous solution was washed with Et₂O (2×20 mL) then acidified with conc. HCl to pH 1 and extracted with CH₂Cl₂ (2×20 mL). The combined organic layers were dried (Na₂SO₄) and evaporated to give the title compound as a white solid (200 mg). ¹H NMR (500 MHz, CDCl₃): δ 9.10 (1H, br s), 8.13 (1H, d, J 8.1), 8.06 (1H, t, J 9.9), 7.91 (2H, s), 7.69 (1H, d, J 7.7), 7.59-7.26 (9H, m), 4.26 (2H, s), 3.86 (3H, s). m/z (ES⁺) 472 [M+H⁺].

### EXAMPLE 101

### Methyl 2-({3-[(1-methyl-1H-imidazol-2-yl)methoxy]-2-phenylquinolin-4-yl}carbonyl)-1-phenylhydrazinecarboxylate

This compound was prepared according to the procedure outlined in Example 97 replacing 2-chloromethyl-1-methyl-triazole hydrochloride with 2-chloromethyl-1-methyl-imidazole hydrochloride. ¹H NMR (500 MHz, DMSO d₆): δ 11.68 (1H, s), 8.10 (1H, d, J 8.3), 7.81 (3H, t, J 6.9), 7.73 (1H, t, J 7.5), 7.53-7.51 (6H, m), 7.42 (1H, s), 7.36 (2H, t, J 7.7), 7.28 (1H, t, J 7.4), 6.43 (1H, s), 4.68 (2H, s), 3.84 (3H, s), 3.01 (3H, s). m/z (ES⁺) 508 [M+H⁺].

### EXAMPLE 102

### Methyl 2-({3-[(1-methyl-1H-1,2,4-triazol-3-yl)methoxy]-2-phenylquinolin-4-yl}carbonyl)-1-phenylhydrazinecarboxylate

This compound was prepared according to the procedure outlined in Example 97 replacing 2-chloromethyl-1-methyl-triazole hydrochloride with 2-chloromethyl-5-methyl-imidazole hydrochloride. ¹H NMR (500 MHz, CDCl₃): δ 11.06 (1H, s), 8.25 (1H, d, J 8.3), 8.13 (1H, d, J 8.2), 7.89 (1H, s), 7.82 (2H, t, J 7.2), 7.71 (1H, t, J 7.7), 7.59-7.51 (6H, m), 7.40 (2H, t, J 7.7), 7.30 (1H, d, J 7.3), 4.86 (2H, s), 3.90 (3H, s), 3.71 (3H, s). m/z (ES⁺) 509 [M+H⁺].

### Description 14: 2-(1,1-Dimethylethyl) 1-Methyl 2-[(3-Methyl-2-phenylquinolin-4-yl)carbonyl]-1-phenylhydrazinedicarboxylate

NaH (60% in mineral oil, 0.2 g, 5 mmol) was added to a solution of methyl 2-[(3-methyl-2-phenylquinolin-4-yl)carbonyl]-1-phenylhydrazinecarboxylate (Description 3, 3.3 g, 8 mmol) in THF (66 mL) and the mixture was stirred at RT until the effervescence had subsided. Di-t-butylpyrocarbonate (1.77 g, 8.12 mmol) was added and the mixture was stirred at 60° C. for 1.25 h. The mixture was cooled and acetic acid (0.7 mL), EtOAc and water were added. The layers were separated and the organic layer was dried (MgSO₄) and the solvent was evaporated under reduced pressure to give the title compound as a foam.

### Description 15: 2-(1,1-Dimethylethyl) 1-Methyl 2-{[3-(Bromomethyl)-2-phenylquinolin-4-yl]carbonyl}-1-phenylhydrazinedicarboxylate

2-(1,1-Dimethylethyl) 1-methyl 2-[(3-methyl-2-phenylquinolin-4-yl)carbonyl]-1-phenylhydrazinedicarboxylate (Description 14, 8 mmol) and NBS (2.2 g, 12.4 mmol)) were suspended in CCl4 (40 mL) and purged thoroughly with nitrogen. The mixture was irradiated with the light from a quartz lamp until the conversion to the product was complete as determined by hplc (3 h.). The mixture was cooled and the solvent was evaporated under reduced pressure. The residue was dissolved in CH₂Cl₂ and filtered through a plug of silica gel, eluting with CH₂Cl₂. The eluent fractions containing product was evaporated under reduced pressure, triturated with ether and cooled to 0° C. The solid was collected and dried in vacuo to give the title compound as a colorless solid (3.7 g, 78%). ¹H NMR (500 MHz, CDCl₃) mixture of rotamers δ 8.44 (0.5H, d, J 8.3), 8.17 (1H, broad s), 7.80 (0.8H, t, J 7.4), 7.75-7.28 (11.2H, m), 5.0-4.6 (0.5H, m), 4.3 (1.3H, s), 4.04 (0.5H, s), 3.92 (2.9H, s), 3.87 (0.8H, s), 0.99-0.90 (9H, m).

### Description 16: Methyl 2-{[3-(Bromomethyl)-2-phenylquinolin-4-yl]carbonyl}-1-phenylhydrazinecarboxylate

2-(1,1-Dimethylethyl) 1-methyl 2-{[3-(bromomethyl)-2-phenylquinolin-4-yl]carbonyl}-1-phenylhydrazinedicarboxylate (Description 15, 0.45 g) was dissolved in TFA (5 mL) and stirred at RT for 1 h. The solvent was evaporated under reduced pressure and the residue was partitioned between aqueous NaHCO₃ (saturated) and EtOAc. The organic layer was dried (MgSO₄) and solvent was evaporated under reduced pressure. The residue was triturated with ether-hexane (1:1) and the solid was collected and dried in vacuo to give the title compound as a colorless solid (0.27 g, 73%). m/z (ES⁺) 492, 490 [M+H⁺].

### Description 17: 8-Fluoro-3-methyl-2-phenylquinoline-4-carboxylic Acid

KOH (88 g, 1560 mmol) in water (400 mL) was added over 30 min. to a suspension of 7-fluoroisatin (64.4 g, 390 mmol) in ethanol (200 mL). Propiophenone (51.9 mL, 390 mmol) was added and the mixture was heated under reflux for 24 h. The mixture was cooled, and hydrochloric acid (conc., 128 mL, 1404 mmol) then acetic acid (11.16 mL, 195 mmol) were added. The mixture was stirred at RT for 16 h. and the solid was collected, washing with water (600 mL), ethanol (200 mL) and hexane/Et₂O (1:1, 400 mL). The solid was flushed with toluene (3×500 mL) and dried in vacuo to give the title compound as an off-white solid (91.44 g, 325 mmol, 83%). ¹H NMR (500 MHz, DMSO-d6) δ 14.4 (1H, br s), 7.70-7.45 (8H, m), 2.40 (3H, s)

### Description 18: 8-Fluoro-3-methyl-N′,2-diphenylquinoline-4-carbohydrazide

Oxalyl chloride (12.47 mLl, 142 mmol) was added dropwise to a suspension of 8-fluoro-3-methyl-2-phenylquinoline-4-carboxylic acid (Description 17, 26.71 g, 95 mmol) in CH₂Cl₂ (320 mL) and DMF (0.5 mL) and the mixture was stirred at RT for 2 h. The solvent was evaporated under reduced pressure and the residue was flushed with toluene (250 mL). The residue was dissolved in CH₂Cl₂ (150 mL) and added dropwise over 30 min. to a stirred mixture of phenylhydrazine (9.81 mL, 100 mmol), potassium carbonate (26.2 g, 190 mmol), water (250 mL) and CH₂Cl₂ (150 mL). The mixture was stirred at RT for 6 h., the layers were separated and the aqueous layer was washed with brine (saturated, 500 mL), dried (MgSO₄), filtered and the solvent was evaporated under reduced pressure. The residue was dissolved in EtOAc (400 mL), washed with hydrochloric acid (1M, 2×400 mL), aqueous NaOH (1M, 2×400 mL) and brine (400 mL), dried (MgSO₄) and the solvent was evaporated under reduced pressure. 2-Propanol (300 mL) was added and the mixture was heated under reflux for 15 min. The mixture was cooled and the solid was collected and dried in vacuo to give the title compound as a cream solid (31.53 g, 89%). m/z (ES⁺) 372 [M+H⁺].

### Description 19: Methyl 2-[(8-Fluoro-3-methyl-2-phenylquinolin-4-yl)carbonyl]-1-phenylhydrazinecarboxylate

Prepared from 8-fluoro-3-methyl-N′,2-diphenylquinoline-4-carbohydrazide (Description 18) according to the method of Description 3. m/z (ES⁺) 430 [M+H⁺].

### Description 20: 2-(1,1-Dimethylethyl) 1-Methyl 2-[(8-Fluoro-3-methyl-2-phenylquinolin-4-yl)carbonyl]-1-phenylhydrazinedicarboxylate

Prepared from methyl 2-[(8-fluoro-3-methyl-2-phenylquinolin-4-yl)carbonyl]-1-phenylhydrazinecarboxylate (Description 19) according to the method of Description 14. m/z (ES⁺) 530 [M+H⁺].

### Description 21: 2-(1,1-Dimethylethyl) 1-Methyl 2-{[8-Fluoro-3-(bromomethyl)-2-phenylquinolin-4-yl]carbonyl}-1-phenylhydrazinedicarboxylate

Prepared from 2-(1,1-dimethylethyl) 1-methyl 2-[(8-fluoro-3-methyl-2-phenylquinolin-4-yl)carbonyl]-1-phenylhydrazinedicarboxylate (Description 20) according to the method of Description 15. m/z (ES⁺) 608, 610 [M+H⁺].

### Description 22: Methyl 2-{[8-Fluoro-3-(Bromomethyl)-2-phenylquinolin-4-yl]carbonyl}-1-phenylhydrazinecarboxylate

Prepared from 2-(1,1-dimethylethyl) 1-Methyl 2-{[8-fluoro-3-(bromomethyl)-2-phenylquinolin-4-yl]carbonyl}-1-phenylhydrazinedicarboxylate (Description 21) according to the method of Description 16. m/z (ES⁺) 508, 510 [M+H⁺].

### Description 23: 5-Fluoro-3-methyl-2-phenylquinoline-4-carboxylic Acid

Prepared from 4-fluorisatin and propiophenone according to the method of Description 17. m/z (ES⁺) 282 [M+H⁺].

### Description 24: 5-Fluoro-3-methyl-N′,2-diphenylquinoline-4-carbohydrazide

Prepared from 5-fluoro-3-methyl-2-phenylquinoline-4-carboxylic acid (Description 23) according to the method of Description 18. m/z (ES⁺) 372 [M+H⁺].

### Description 25: Methyl 2-[(5-Fluoro-3-methyl-2-phenylquinolin-4-yl)carbonyl]-1-phenylhydrazinecarboxylate

Prepared from 5-fluoro-3-methyl-N′,2-diphenylquinoline-4-carbohydrazide (Description 24) according to the method of Description 3. m/z (ES⁺) 430 [M+H⁺].

### Description 26: 2-(1,1-Dimethylethyl) 1-Methyl 2-[(5-Fluoro-3-methyl-2-phenylquinolin-4-yl)carbonyl]-1-phenylhydrazinedicarboxylate

Prepared from methyl 2-[(5-fluoro-3-methyl-2-phenylquinolin-4-yl)carbonyl]-1-phenylhydrazinecarboxylate (Description 25) according to the method of Description 14. m/z (ES⁺) 530 [M+H⁺].

### Description 27: 2-(1,1-Dimethylethyl) 1-Methyl 2-{[5-Fluoro-3-(bromomethyl)-2-phenylquinolin-4-yl]carbonyl}-1-phenylhydrazinedicarboxylate

Prepared from 2-(1,1-dimethylethyl) 1-methyl 2-[(5-fluoro-3-methyl-2-phenylquinolin-4-yl)carbonyl]-1-phenylhydrazinedicarboxylate (Description 26) according to the method of Description 15. m/z (ES⁺) 608/610 [M+H⁺].

### Description 28: Methyl 2-{[5-Fluoro-3-(Bromomethyl)-2-phenylquinolin-4-yl]carbonyl}-1-phenylhydrazinecarboxylate

Prepared from 2-(1,1-dimethylethyl) 1-methyl 2-{[5-fluoro-3-(bromomethyl)-2-phenylquinolin-4-yl]carbonyl}-1-phenylhydrazinedicarboxylate (Description 27) according to the method of Description 16. m/z (ES⁺) 508/510 [M+H⁺].

### Description 29: 7-Bromo-3-methyl-2-phenylquinoline-4-carboxylic Acid

Prepared from 7-bromoisatin and propiophenone according to the method of Description 17. ¹H NMR (400 MHz, DMSO-d6) δ 8.27 (1H, d, J 1.8), 7.82 (1H, dd, J 1.9, 8.9), 7.76 (1H, d, J 8.9), 7.61 (2H, dd, J 2.0, 7.9), 7.56-7.50 (3H, m), 2.37 (3H, s).

### Description 30: 7-Bromo-3-methyl-N′,2-diphenylquinoline-4-carbohydrazide

Prepared from 7-bromo-3-methyl-2-phenylquinoline-4-carboxylic acid (Description 29) according to the method of Description 18. ¹H NMR (400 MHz, CDCl₃) δ 8.34-8.31 (1H, m), 7.94 (1H, br s), 7.70-7.62 (2H, m), 7.50-7.42 (5H, m), 7.31 and 7.18 (2H, two t, J 7.9), 7.02-6.88 (3H, m), 6.69 and 6.55 (1H, two m), 2.43 and 2.32 (3H, two s).

### Description 31: Methyl 2-[(7-Bromo-3-methyl-2-phenylquinolin-4-yl)carbonyl]-1-phenylhydrazinecarboxylate

Prepared from 7-bromo-3-methyl-N′,2-diphenylquinoline-4-carbohydrazide (Description 30) according to the method of Description 3. m/z (ES⁺) 490/492 [M+H⁺].

### Description 32: 2-(1,1-Dimethylethyl) 1-Methyl 2-[(7-Bromo-3-methyl-2-phenylquinolin-4-yl)carbonyl]-1-phenylhydrazinedicarboxylate

Prepared from methyl 2-[(7-bromo-3-methyl-2-phenylquinolin-4-yl)carbonyl]-1-phenylhydrazinecarboxylate (Description 31) according to the method of Description 14. m/z (ES⁺) 490/492 [M-Boc+2H⁺].

### Description 33: 2-(1,1-Dimethylethyl) 1-Methyl 2-{[7-Bromo-3-(bromomethyl)-2-phenylquinolin-4-yl]carbonyl}-1-phenylhydrazinedicarboxylate

Prepared from 2-(1,1-dimethylethyl) 1-methyl 2-[(7-bromo-3-methyl-2-phenylquinolin-4-yl)carbonyl]-1-phenylhydrazinedicarboxylate (Description 32) according to the method of Description 15. m/z (ES⁺) 668/670/672 [M+H⁺].

### Description 34: Methyl 2-{[7-Bromo-3-(Bromomethyl)-2-phenylquinolin-4-yl]carbonyl}-1-phenylhydrazinecarboxylate

Prepared from 2-(1,1-dimethylethyl) 1-methyl 2-{[7-bromo-3-(bromomethyl)-2-phenylquinolin-4-yl]carbonyl}-1-phenylhydrazinedicarboxylate (Description 33) according to the method of Description 16. m/z (ES⁺) 568/570/572 [M+H⁺].

### Description 35: 3-(5,6-Dihydro[1,2,4]triazolo[1,5-a]pyrazin-7(8H)-ylmethyl)-2-phenylquinoline-4-carboxylic Acid

A mixture of 3-(bromomethyl)-2-phenylquinoline-4-carboxylic acid (Description 5, 3.4 g, 10.3 mmol), 5,6,7,8-tetrahydro-[1,2,4]triazolo[1,5-a]pyrazine (1.24 g, 9.1 mmol) and triethylamine (1.39 mL, 1.01 g, 10 mmol) in DMF (50 mL) was heated at 60° C. for 40 h. The mixture was cooled and the solvent was evaporated under reduced pressure. EtOAc (500 mL) was added and the mixture was heated to reflux, filtered and cooled. The solvent was evaporated under reduced pressure to a volume of 100 mL and the solid was collected and dried in vacuo to give the title compound as a solid (1.0 g, 28%). m/z (ES⁺) 386 [M+H⁺].

### Description 36: 2-Phenyl-3-(2-propenyl)quinoline-4-carboxylic Acid

Prepared from isatin and 1-phenyl-4-penten-1-one according to the method of Description 17.

### Description 37: N′,2-Diphenyl-3-(2-propenyl)quinoline-4-carbohydrazide

Prepared from 2-phenyl-3-(2-propenyl)quinoline-4-carboxylic acid (Description 36) according to the method of Description 18.

### Description 38: Methyl 2-{[2-Phenyl-3-(2-propenyl)quinolin-4-yl]carbonyl}-1-phenylhydrazinecarboxylate

Prepared from N′,2-diphenyl-3-(2-propenyl)quinoline-4-carbohydrazide (Description 37) according to the method of Description 3.

### Description 39: 2-(1,1-Dimethylethyl) 1-Methyl 2-{[2-Phenyl-3-(2-propenyl)quinolin-4-yl]carbonyl}-1-phenylhydrazinedicarboxylate

Prepared from methyl 2-{[2-phenyl-3-(2-propenyl)quinolin-4-yl]carbonyl}-1-phenylhydrazinecarboxylate (Description 38) according to the method of Description 14.

### Description 40: 2-(1,1-Dimethylethyl) 1-Methyl 2-{[3-(2-Oxoethyl)-2-phenylquinolin-4-yl]carbonyl}-1-phenylhydrazinedicarboxylate

Ozone was bubbled through a stirred, cooled (−78° C.) solution of 2-(1,1-dimethylethyl) 1-methyl 2-{[2-phenyl-3-(2-propenyl)quinolin-4-yl]carbonyl}-1-phenylhydrazinedicarboxylate (Description 39, 200 mg, 0.46 mmol) in CH₂Cl₂/methanol (9:1, 50 mL) until a blue color persisted. The mixture was flushed with oxygen, then nitrogen and dimethylsulfide (0.5 mL) was added. The mixture was allowed to warm to RT, washed with aqueous NaHCO₃ (saturated) and brine, dried (MgSO₄) and the solvent was evaporated under reduced pressure. The residue was purified by column chromatography on silica gel, eluting with 20% EtOAc in isohexanes, to give the title compound (100 mg). m/z (ES⁺) 540 [M+H⁺].

### Description 41: 2-(1,1-Dimethylethyl) 1-Methyl 2-[{3-[2-(4-Hydroxy)piperidinoethyl]-2-phenylquinolin-4-yl}carbonyl]-1-phenylhydrazinedicarboxylate

Prepared from 2-(1,1-dimethylethyl) 1-methyl 2-{[3-(2-oxoethyl)-2-phenylquinolin-4-yl]carbonyl}-1-phenylhydrazinedicarboxylate (Description 40, 100 mg, 0.185 mmol) and 4-piperidinol according to the method of Description 61.

### Description 42: Methyl 3-hydroxy-2-phenylquinoline-4-carboxylate

Sulfuric acid (conc., 6.0 mL) was added to a suspension of 3-hydroxy-2-phenylquinoline-4-carboxylic acid (15.0 g, 56.5 mmol) in methanol (100 mL) and the mixture was heated under reflux for 96 h. The mixture was cooled and the solvent was evaporated under reduced pressure. Water (200 mL) and CH₂Cl₂ (200 mL) were added, the aqueous layer was basified with solid sodium carbonate and the layers were separated. The aqueous layer was extracted with CH₂Cl₂ (4×) and the combined organic fractions dried (Na₂SO₄) and the solvent was evaporated under reduced pressure to give the title compound as a pale yellow solid. m/z (ES⁺) 280 [M+H⁺]

### Description 43: Methyl 3-[1-(1,1-Dimethylethyl)piperidin-4-yloxy]-2-phenylquinoline-4-carboxylate

Di-t-butyl azodicarboxylate (11.05 g, 48.0 mmol) and triphenylphosphine (12.58 g, 48.0 mmol) were added to a stirred solution of methyl 3-hydroxy-2-phenylquinoline-4-carboxylate (Description 42, 6.7 g, 24.0 mmol) and 1-(1,1-dimethylethyl)-4-piperidinol (4.53 g, 28.8 mmol) in THF (60 mL) and the mixture was stirred at RT for 72 h. Water (100 mL) was added and the mixture was extracted with EtOAc (3×100 mL). The combined organic fractions were washed with water (2×100 mL), brine (2×100 mL), dried (Na₂SO₄), and the solvent was evaporated under reduced pressure to give the title compound as an orange oil (4.2 g, 42%). m/z (ES⁺) 419 [M+H⁺]

### Description 44: 3-[1-(1,1-Dimethylethyl)piperidin-4-yloxy]-2-phenylquinoline-4-carboxylic Acid

KOH (2.68 g, 47.8 mmol) was added to a solution of methyl 3-[1-(1,1-dimethylethyl)piperidin-4-yloxy]-2-phenylquinoline-4-carboxylate (Description 43, 4.0 g, 9.56 mmol) in methanol (60 mL) and the mixture was heated under reflux for 72 h. The mixture was cooled and the solvent was evaporated under reduced pressure. Water (50 mL) and EtOAc (50 mL) were added and the aqueous phase was adjusted to pH 5 with acetic acid. The layers were separated and the aqueous layer was extracted with EtOAc (4×50 mL). The combined organic fractions were dried (Na₂SO₄), and the solvent was evaporated under reduced pressure to give the title compound as a pale brown solid (3.8 g, 98%). m/z (ES⁺) 405 [M+H⁺]

### Description 45: Methyl 3-[1-(1,1-Dimethylethyloxycarbonyl)piperidin-4-yloxy]-2-phenylquinoline-4-carboxylate

Prepared from 1,1-dimethylethyl 4-hydroxypiperidinecarboxylate and methyl 3-hydroxy-2-phenylquinoline-4-carboxylate according to the method of Description 43. m/z (ES⁺) 463 [M+H⁺]

### Description 46: 3-[1-(1,1-Dimethylethyloxycarbonyl)piperidin-4-yloxy]-2-phenylquinoline-4-carboxylic Acid

Prepared from methyl 3-[1-(1,1-dimethylethyloxycarbonyl)piperidin-4-yloxy]-2-phenylquinoline-4-carboxylate (Description 45) according to the method of Description 44. m/z (ES⁺) 449 [M+H⁺]

### Description 47: 1,1-Dimethylethyl 4-[(1E)-3-Oxo-3-phenyl-1-propenyl]-1-piperidinecarboxylate

To a solution of 4-piperidinemethanol (11.5 g, 0.1 mol) in CH₂Cl₂ (200 mL) was added di-t-butyldicarbonate (23.98 g, 0.11 mol) and the mixture was stirred at RT for 16 h. The solvent was removed by evaporation and the resultant solid was dried under vacuum for 3 h.

To a cooled (−60° C.) solution of DMSO (17.2 mL) in CH₂Cl₂ (55 mL) was slowly added a solution of oxalyl chloride (10.2 mL) in CH₂Cl₂ (140 mL). After stirring the cloudy solution at −60° C. for 20 min, a solution of 1-(1,1-dimethylethyl)carbonyl-4-piperidinemethanol (prepared above) in dichloromethane (55 mL) was added over 20 min. and then the mixture was stirred at −60° C. for an additional 20 min. Triethylamine (70 mL) was added and the solution was allowed to warm to RT. Water (100 mL) was added and the organic phase was washed with aqueous citric acid (1M, 2×100 mL), water and saturated brine, dried (MgSO₄). Removal of the solvent by evaporation gave 1-(1,1-dimethylethyl)carbonyl-4-piperidinecarboxaldehyde as an oil (23.9 g).

To a cooled (−78° C.) solution of LHMDS (1M in THF, 87 mL, 87 mmol) in THF (100 mL) was added a solution of acetophenone (10.44 g, 87 mmol) in THF (30 mL). After stirring the solution at −78° C. for 1 h., a solution of 1-(1,1-dimethylethyl)carbonyl-4-piperidinecarboxaldehyde (18.6 g, 87 mmol) in THF (50 mL) was added and the mixture was stirred at −78° C. for 30 min. then allowed to warm to −30° C. Aqueous citric acid (1M, 200 mL) was added and the mixture was warmed to RT. EtOAc (400 mL) was added and organic phase was washed with water and brine, dried (MgSO₄) and the solvent was evaporated under reduced pressure. The residue was dissolved in CH₂Cl₂ (200 mL), cooled in an ice bath and triethylamine (24.2 mL, 174 mmol) was added. Methanesulfonyl chloride (8 mL) was slowly added and the mixture was stirred at 0° C. for 60 min. and then heated under reflux for 30 min. To the cooled solution was added CH₂Cl₂ (200 mL) and saturated aqueous NaHCO₃ solution and the solution was stirred at RT for 30 min. The layers were separated and the organic phase was dried (MgSO₄) and the solvent was evaporated under reduced pressure. The residue was purified by chromatography on silica gel eluting with increasing amounts of EtOAc in isohexane (10-30%) to give the title compound as an oil (21.6 g, 78%). ¹H NMR (500 MHz, CDCl₃): δ 7.92 (2H, t, J 7.1), 7.58-7.46 (3H, m), 6.99 (1H, dd, J 6.5, 15.6), 6.87 (1H, dd, J 1.0, 15.5), 4.14-4.10 (2H, m), 2.80 (2H, m), 2.44-2.38 (1H, m), 1.80 (2H, d, J 12.3), 1.46 (9H, s), 1.46-1.40 (2H, m).

### Description 48: 1,1-Dimethylethyl 4-(3-Oxo-3-phenyl-1-propyl-1-piperidinecarboxylate

A mixture of 1,1-dimethylethyl 4-[(1E)-3-oxo-3-phenyl-1-propenyl]-1-Piperidinecarboxylate (Description 47, 21.1 g, 67 mmol) and 10% palladium on carbon (1.9 g) in EtOAc (300 mL) was shaken under hydrogen (30 psi) for 6 h. The mixture was filtered and the solvent was evaporated under reduced pressure to give the title compound as an oil (20.9 g). ¹H NMR (500 MHz, CDCl₃): δ 7.97-7.91 (2H, d, J 7.8), 7.58-7.52 (1H, t), 7.46 (2H, t, J 7.6), 4.12 (2H, m), 3.00 (2H, t, J 7.5), 2.68 (3H, m), 1.70 (4H, m), 1.42 (10H, m), 1.26 (2H, t, J 7.1).

### Description 49: 3-({1-[(1,1-Dimethylethoxy)carbonyl]piperidin-4-yl}methyl-2-phenylquinoline-4-carboxylic Acid

1,1-Dimethylethyl 4-(3-oxo-3-phenyl-1-propyl-1-piperidinecarboxylate (Description 48, 20.9 g, 65.9 mmol) was added to a solution of isatin (9.69 g, 65.9 mmol) in ethanol (68 mL) and KOH (14.76 g, 264 mmol) dissolved in water (68 mL). The solution was heated at 100° C. for 5 days, cooled to RT and diluted with water (300 mL). The mixture was washed with Et₂O (2×100 mL), neutralized by addition of acetic acid (16 mL) and extracted with EtOAc (5×100 mL). The combined organic fractions were dried (MgSO₄) and the solvent was evaporated under reduced pressure. The residue was triturated with Et₂O (20 mL) and the solid was collected and dried in vacuo to give the title compound (5.6 g). m/z 447 (M+H).

### Description 50: 3-({1-[(Phenylmethoxy)carbonyl]piperidin-4-yl}methyl)-2-phenylquinoline-4-carboxylic Acid

3-({1-[(1,1-Dimethylethoxy)carbonyl]piperidin-4-yl}methyl-2-phenylquinoline-4-carboxylic Acid (Description 49, 1.2 g) was dissolved in TFA (5 mL) and stirred at RT for 30 min. The solvent was evaporated under reduced pressure and the residue was dissolved in CH₂Cl₂ (10 mL) and aqueous potassium carbonate (10%, 10 mL) and benzyl chloroformate (0.52 mL) were added. The mixture was stirred at RT for 1 h, then the CH₂Cl₂ was evaporated under reduced pressure. The mixture was extracted EtOAc (3×30 mL). and the combined organic fractions were dried (MgSO₄) and the solvent was evaporated under reduced pressure to give the title compound as an oil (1.1 g).

### Description 51: 3-({1-[(1,1-Dimethylethoxy)carbonyl]piperidin-4-yl}methyl)-N′,2-diphenylquinoline-4-carbohydrazide

Oxalyl chloride (0.038 mL) was added slowly to a solution of DMF (0.5 mL) in CH₂Cl₂ (5 mL) and the mixture was stirred at RT for 20 min. 3-({1-[(1,1-Dimethylethoxy)carbonyl]piperidin-4-yl}methyl-2-phenylquinoline-4-carboxylic acid (Description 49, 100 mg, 0.224 mmol) in CH₂Cl₂ (5 mL) was added and the mixture was stirred at RT for 1.5 h. The CH₂Cl₂ was evaporated under reduced pressure and the residue was quickly partitioned between EtOAc and aqueous NaHCO₃ (saturated). The organic layer was washed with water (3×) and brine, dried (MgSO₄) and the solvent evaporated under reduced pressure. The residue was dissolved in CH₂Cl₂ (5 mL) and phenylhydrazine (48 mg) was added. The mixture was stirred at RT for 16 h, then the solvent evaporated under reduced pressure. The residue was partitioned between EtOAc and aqueous citric acid (10%). The organic fraction was dried (MgSO₄) and the solvent evaporated under reduced pressure. The residue was purified by column chromatography on silica gel, eluting with 10%-40% EtOAc in hexane, to give the title compound (42 mg). m/z (ES⁺) 537 [M+H⁺].

### Description 52: Methyl 2-{[3-({1-[(1,1-Dimethylethoxy)carbonyl]piperidin-4-yl}methyl)-2-phenylquinolin-4-yl]carbonyl}-1-phenylhydrazinecarboxylate

Prepared from 3-({1-[(1,1-dimethylethoxy)carbonyl]piperidin-4-yl}methyl)-N′,2-diphenylquinoline-4-carbohydrazide (Description 51) according to the method of Description 3. m/z (ES⁺) 595 [M+H⁺].

### Description 53: 3-({1-[(Phenylmethoxy)carbonyl]piperidin-4-yl}methyl)-N′,2-diphenylquinoline-4-carbohydrazide

Prepared from 3-({1-[(phenylmethoxy)carbonyl]piperidin-4-yl}methyl)-2-phenylquinoline-4-carboxylic acid (Description 50) according to the method of Description 18. m/z (ES⁺) 571 [M+H⁺].

### Description 54: Methyl 2-{[3-({1-[(Phenylmethethoxy)carbonyl]piperidin-4-yl}methyl)-2-phenylquinolin-4-yl]carbonyl}-1-phenylhydrazinecarboxylate

Prepared from 3-({1-[(phenylmethoxy)carbonyl]piperidin-4-yl}methyl)-N′,2-diphenylquinoline-4-carbohydrazide (Description 53) according to the method of Description 3. m/z (ES⁺) 629 [M+H⁺].

### Description 55: Methyl 2-({3-[(piperidin-4-yl)methyl]-2-phenylquinolin-4-yl}carbonyl)-1-phenylhydrazinecarboxylate

Palladium on carbon (10%, 60 mg) was added to a solution of methyl 2-{[3-({1-[(phenylmethethoxy)carbonyl]piperidin-4-yl}methyl)-2-phenylquinolin-4-yl]carbonyl}-1-phenylhydrazinecarboxylate (Description 54, 130 mg) and acetic acid (6 mL) in EtOAc (20 mL) and the mixture was stirred under hydrogen (1 atm.) for 72 h. The mixture was filtered, the solvent was evaporated under reduced pressure and the residue was purified by preparative HPLC to give the title compound. m/z (ES⁺) 495 [M+H⁺].

### Description 56: Methyl 2-{[3-(Azidomethyl)-2-phenylquinolin-4-yl]carbonyl}-1-phenylhydrazinecarboxylate

Sodium azide (0.1593 g, 2.45 mol) was added to a solution of methyl 2-{[3-(bromomethyl)-2-phenylquinolin-4-yl]carbonyl}-1-phenylhydrazinecarboxylate (Description 16) (1.20 g, 2.45 mmol) in anhydrous DMF (50 mL) and the mixture was stirred at RT for 5 h. The mixture was partitioned between water (200 mL) and EtOAc (200 mL). The organic layer was washed with brine (50 mL), dried (MgSO₄) and evaporated to give the title compound.

### Description 57: 1-(3-Fluorophenyl)-1-phenylhydrazine

A slurry solution of 1-bromo-3-fluorobenzene (0.32 mL, 2.86 mmol), phenyl hydrazine (0.33 mL, 3.43 mmol) and sodium t-butoxide (0.38 g, 4.01 mmol) in diisopropylamine (12 mL) was degassed by bubbling nitrogen through for 15 min. Palladium acetate (0.032 g, 0.143 mmol) and (1,1′-binaphthalene)-2,2′-diylbis(diphenylphosphine) (0.089 g, 0.143 mmol) were added and the mixture was heated at 80° C. for 4 h. The mixture was cooled, diluted with Et₂O, filtered through Celite and the solvent was evaporated under reduced pressure. The residue was purified by flash chromatography on silica gel eluting with isohexane/CH₂Cl₂ (50:50) to give the title compound (0.41 g, 70%). ¹H NMR (400 MHz, CDCl₃) δ 7.34 (2H, dd, J 8.4, 15.8), 7.27 (2H, d, J 1.2), 7.19-7.07 (2H, m), 6.93-6.87 (2H, m), 6.59-6.55 (1H, m), 4.15 (2H, br s).

### Description 58: 1,1-Bis(3-fluorophenyl)hydrazine

Prepared from 3-fluorophenylhydrazine hydrochloride according to the method of Description 57. m/z (ES⁺) 221 [M+H⁺].

### Description 59: 3-(1-Phenylhydrazino)pyridine

Prepared from 3-bromopyridine and phenylhydrazine according to the method of Description 57. m/z (ES⁺) 186 [M+H⁺].

### Description 60: 2-(1-Phenylhydrazino)-1,3-thiazole

NaH (60% dispersion in mineral oil, 0.92 g, 23.1 mmol) was added to a solution of phenylhydrazine (0.50 g, 4.62 mmol) in 1,4-dioxane (8.0 mL) and the mixture was stirred at RT for 20 min. 2-Bromo-1,3-thiazole (0.41 mL, 4.62 mmol) was added and the mixture was heated under reflux for 12 h. The mixture was cooled and the solvent was evaporated under reduced pressure. The residue was purified by flash chromatography on silica gel eluting with isohexane/EtOAc (70:30) to give the title compound (0.19 g, 21%). m/z (ES⁺) 192 [M+H⁺].

### Description 61: N-(Tetrahydro-2H-pyran-4-yl)-4-piperidinamine

Acetic acid (0.5 mL) and sodium triacetoxyborohydride (4.2 g, 20.0 mmol) were added to a solution of 1-(phenylmethyl)-4-piperidinamine (1.9 g, 10.0 mmol) and tetrahydro-4H-pyran-4-one (1.0 g, 10.0 mmol) in CH₂Cl₂ (50 mL) and the mixture was stirred at RT for 18 h. Water (50 mL) was added, the layers were separated and the aqueous layer was extracted with chloroform/1-propyl alcohol (90/10, 4×50 mL). The combined organic fractions were dried (Na₂SO₄), and the solvent was evaporated under reduced pressure. The residue was dissolved in ethanol (30 mL), palladium on carbon (10%, 300 mg) was added and the mixture was shaken under hydrogen (45 psi) for 48 h. The mixture was filtered and the solvent was evaporated under reduced pressure to give the title compound (1.14 g, 95%) as a colorless solid. m/z (ES⁺) 185 [M+H⁺].

### Description 62: 1,1-Dimethylethyl 3-(1-Hydroxy-1-methylethyl)-1-piperidinecarboxylate

Methyl magnesium bromide (1.4M in toluene/THF 75:25, 31.8 mL, 44.5 mmol) was added slowly to a stirred, cooled (−78° C.) solution of 3-ethyl 1-(1,1-dimethylethyl) 1,3-piperidinedicarboxylate (5.2 g, 20.2 mmol) in THF (30 mL) and the mixture was allowed to warm to RT and stirred for 1 h. Aqueous ammonium chloride (saturated, 10 mL) was added and the mixture was partitioned between aqueous ammonium chloride (saturated, 50 mL) and EtOAc (2×100 mL). The combined organic fractions were dried (MgSO₄) and the solvent was evaporated under reduced pressure to give the title compound as a colorless oil (4.67 g, 95%).

### Description 63: α,α-Dimethyl-3-piperidinemethanol

Methanolic hydrogen chloride (1M, 8 mL) was added to 1,1-dimethylethyl 3-(1-hydroxy-1-methylethyl)-1-piperidinecarboxylate (Description 62, 1.0 g, 4.1 mmol) in methanol (8 mL) and the mixture was stirred at RT overnight. The solvent was evaporated under reduced pressure and the residue was dissolved in methanol and applied to a Bond Elut™ cartridge (SCX, 10 g). The cartridge was washed with MeOH and then eluted with methanolic ammonia (2M, 50 mL). The solvent was under reduced pressure to give the title compound (350 mg, 60%); m/z (ES⁺) 144 [M+H⁺].

### Description 64: 1-(1,1-Dimethylethyl) 4-piperidinedicarboxylate Anhydride with 1H-Imidazole-1-carboxylic Acid

Carbonyldiimidazole (39.4 g, 243 mmol) was added in portions to a stirred solution of 1-(1,1-dimethylethyl) 4-piperidinedicarboxylate (42.5 g, 187 mmol) in CH₂Cl₂ (550 mL) and the mixture was stirred at 4° C. overnight. The mixture was poured into Et₂O (2 L) and ice water (2 L), the layers were separated and the organic layer was washed with cold aqueous NaHCO₃ (saturated, 500 mL) and brine (500 mL), dried (MgSO₄) and the solvent was evaporated under reduced pressure to give the title compound as a colorless solid (52 g).

### Description 65: 1,1-Dimethylethyl 4-(1,3-Dioxobutyl)piperidinecarboxylate

Acetone (41 mL) in THF (300 mL) was added dropwise to a stirred suspension of KH (30% in mineral oil, 75 g, 561 mmol) in THF (1.4 L) at RT. The mixture was cooled to −78° C. and treated with 1-(1,1-dimethylethyl) 4-piperidinedicarboxylate anhydride with 1H-imidazole-1-carboxylic acid (Description 64, 52 g) in THF (500 mL). The mixture was allowed to warm to RT and stirred overnight, then ethanol (50 mL) was added dropwise over 5 min. The mixture was poured into hydrochloric acid (1N, 2 L) and extracted with Et₂O (2×1 L). The combined organic fractions were washed with aqueous NaHCO₃ (saturated, 500 mL) and brine (500 mL), dried (MgSO₄) and the solvent was evaporated under reduced pressure. Isohexane (1 L) and MeCN (500 mL) were added, the layers were separated and the isohexane layer was extracted with MeCN (500 mL). The MeCN layers were combined and the solvent was evaporated under reduced pressure. The residue was filtered through a silica pad, eluting with 0-20% EtOAc in hexanes, to give the title compound as a colorless solid (36 g, 72%). ¹H NMR (CDCl₃) δ 5.50 (1H, s), 4.18-4.11 (2H, m), 2.79-2.62 (2H, m), 2.34-2.27 (1H, m), 2.07 (3H, s), 1.80 (2H, brd, J 14.3), 1.60-1.51 (2H, m), 1.46 (9H, s).

### Description 66: 1,1-Dimethylethyl 4-[1-(1,1-Dimethylethyl)-3-methyl-1H-pyrazol-5-yl]piperidinecarboxylate

(1,1-Dimethylethyl)hydrazine hydrochloride (14.8 g, 118 mmol) then triethylamine (17 mL, 120 mmol) were added to a solution of 1,1-dimethylethyl 4-(1,3-dioxobutyl)piperidinecarboxylate (Description 65, 8 g, 29.7 mmol) in ethanol (200 mL) and the mixture was stirred at RT overnight. The solvent was evaporated under reduced pressure and the residue was suspended in EtOAc (1 L), washed with aqueous NaOH (1N, 500 mL), water (500 mL) and brine (500 mL), dried (MgSO₄) and the solvent was evaporated under reduced pressure to give the title compound as a yellow oil (9.5 g). ¹H NMR (CDCl₃) δ 5.87 (1H, s), 4.27-4.18 (2H, m), 3.09-2.99 (1H, m), 2.79-2.68 (2H, m), 2.20 (3H, s), 1.85 (2H, brd, J 13.3), 1.63 (9H, s), 1.61-1.52 (2H, m), 1.48 (9H, s).

### Description 67: 4-[1-(1,1-Dimethylethyl)-3-methyl-1H-pyrazol-5-yl]piperidine

Methanolic hydrogen chloride (1N, 100 mL) was added to a solution of 1,1-dimethylethyl 4-[1-(1,1-dimethylethyl)-3-methyl-1H-pyrazol-5-yl]piperidinecarboxylate (Description 66, 9.5 g, 29 mmol) in methanol (100 mL) and the mixture was stirred at RT for 4 h. The solvent was evaporated under reduced pressure and the residue was triturated with EtOAc. The solid was collected, suspended in aqueous NaOH (4N, 20 mL) and extracted with CH₂Cl₂ (2×100 mL). The combined organic fractions were dried (MgSO₄) and the solvent was evaporated under reduced pressure to give the title compound (6.4 g). ¹H NMR (CDCl₃) δ 5.91 (1H, s), 3.17 (2H, brd, J 13.0), 3.03 (1H, tt, J 13.0 and 4.3), 2.69 (2H, dt, J 12.8 and 3.5), 2.21 (3H, s), 1.86 (2H, brd, J 13.4), 1.67 (9H, s), 1.68-1.56 (2H, m).

### Description 68: 4-[1-(-(2,2,2-Trifluoroethyl))-3-methyl-1H-pyrazol-5-yl]piperidine

Prepared from 1,1-dimethylethyl 4-(1,3-dioxobutyl)piperidinecarboxylate (Description 65) according to the methods of Description 66 and Description 67, substituting trifluoroethylhydrazine oxalate for (1,1-dimethylethyl)hydrazine hydrochloride. ¹H NMR (CDCl₃) δ 5.91 (1H, s), 4.58 (2H, dd, J 16.4 and 8.6), 3.17 (2H, brd, J 13.2), 2.70 (2H, dt, J 12.5 and 2.4), 2.60 (1H, tt, J 12.1 and 4.1), 2.23 (3H, s), 1.82 (2H, brd, J 14.4), 1.58 (2H, qd, J 12.5 and 3.8).

The following compounds were prepared from 1,1-dimethylethyl 1-piperazinecarboxylate and suitable ketones according to the methods of Description 61 and Description 67.

| | | | | | m/z |
|---|---|---|---|---|---|
| Desc. | Ketone | Product | Formula | m. wt. | (ES⁺) |
| 69 | bicyclo[3.3.1]nonane-9-one | 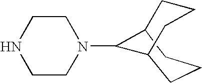 | C₁₃H₂₄N₂ | 208.34 | 209 |
| 70 | tetrahydro-4H-thiopyran-4-one | 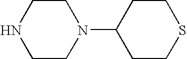 | C₉H₁₈N₂S | 186.32 | 187 |
| 71 | 2-methyltetra-hydro-3-furanone | 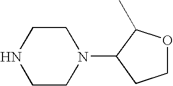 | C₉H₁₈N₂O | 170.25 | 171 |
| 72 | 3-methyl-tetrahydro-4H-pyran-4-one | 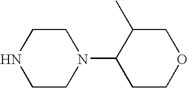 | C₁₀H₂₀N₂O | 184.258 | 185 |
| 73 | tetrahydro-4H-pyran-3-one | 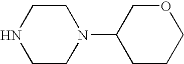 | C₉H₁₈N₂O | 170.25 | 171 |

### Description 74: 1,1-Dimethylethyl 4-[Tetrahydro-2H-thiopyran-4-yl]-1-piperazinecarboxylate

Prepared from 1,1-dimethylethyl 1-piperazinecarboxylate and tetrahydro-4H-thiopyran-4-one according to the method of Description 61.

### Description 75: 1-[Tetrahydro-1,1-dioxido-2H-thiopyran-4-yl]-1-piperazine Hydrochloride

4-Methylmorpholine-N-oxide (3.0 g, 25.6 mmol) and osmium tetroxide (2% in t-butanol, 1 mL) were added to a solution of 1,1-dimethylethyl 4-[tetrahydro-2H-thiopyran-4-yl]-1-piperazinecarboxylate (Description 74, 7.8 g, 27 mmol) in THF (30 mL) and the mixture was stirred at RT for 72 h. The mixture was poured into water (30 mL), extracted with EtOAc (3×30 mL) and the combined organic fractions were dried (Na₂SO₄), and the solvent was evaporated under reduced pressure. The residue was dissolved in methanolic hydrogen chloride (1M, 100 mL) and the mixture was stirred for at RT for 20 h. The solvent was evaporated under reduced pressure and the residue was triturated with cold iso-hexane. The solid was collected and dried in vacuo to give the title compound as a colorless solid (2.56 g, 43%). m/z (ES⁺) 219 [M+H⁺]

### Description 76: 1,1-Dimethylethyl 4-(4-Methyltetrahydro-2H-pyran-4-yl)piperazine-1-carboxylate

A mixture of tetrahydro-4H-pyran-4-one (25.0 g, 250 mmol), 1,1-dimethylethyl 1-piperazinecarboxylate (51.2 g, 275 mmol) and 1,2,3-triazole (20.7 g, 300 mmol) in toluene (200 mL) was heated under reflux with azeotropic removal of water overnight. The mixture was allowed to cool to RT and was then added over 40 min. via cannula to a solution of methyl magnesium chloride (3M in THF, 333 mL, 1 mol), keeping the internal temperature below 24° C., and the mixture was stirred at RT for 3 h. The mixture was poured slowly onto aqueous ammonium chloride solution (20%, 500 mL), keeping the temperature below 30° C. The layers was separated and the aqueous layer was extracted with EtOAc. The combined organic fractions were washed with aqueous NaOH (2M, 400 mL) and water (400 mL), dried (MgSO₄) and the solvent was evaporated under reduced pressure to give an orange oil (70 g). A sample (5 g) was purified by column chromatography on silica gel, eluting with CH₂Cl₂ (containing 1% ammonia) containing a gradient of methanol (0-3%), to give the title compound as a pale yellow oil (2.3 g, 81%); m/z (ES⁺) 285 [M+H⁺].

### Description 77: 1-(4-Methyltetrahydro-2H-pyran-4-yl)piperazine

Prepared from 1,1-dimethylethyl 4-(4-methyltetrahydro-2H-pyran-4-yl)piperazine-1-carboxylate (Description 76) according to the method of Description 67. m/z (ES⁺) 185 [M+H⁺].

### Description 78: 1-(1-Methylcyclohex-1-yl)piperazine

Prepared from cyclohexanone and 1,1-dimethylethyl 1-piperazinecarboxylate according to the methods of Description 76 and Description 67. m/z (ES⁺) 183 [M+H⁺].

### Description 79: α,α-Dimethyl-4-[(phenylmethoxy)carbonyl]-1-piperazineacetic Acid

A solution of 2-bromo-2-methylpropionic acid (21.47 g, 128.6 mmol) in toluene (50 mL) was added dropwise in the dark over 30 min. to a mixture of phenylmethyl 1-piperazinecarboxylate (28 g, 127.3 mmol), triethylamine (71 mL, 509.2 mmol), Hyflo™ (30 g) and silver (I) oxide (29.5 g, 127.3 mmol) in toluene (100 mL) and the mixture was stirred at RT overnight. The mixture was filtered through a pad of Hyflo™, washing with toluene. The solvent was evaporated under reduced pressure to give the title compound. m/z (ES⁺) 307 [M+H⁺].

### Description 80: Phenylmethyl 4-(2-Hydroxy-1,1-dimethylethyl)-1-piperazinecarboxylate

Isobutyl chloroformate (1 mL, 8 mmol) was added to a mixture of α,α-dimethyl-4-[(phenylmethoxy)carbonyl]-1-piperazineacetic acid (Description 79, 2 g, 7.35 mmol) and triethylamine (2 mL, 14.7 mmol) in THF (50 mL) and the mixture was stirred at RT for 90 min. The mixture was filtered through Hyflo™ and sodium borohydride (1.4 g) in water (50 mL) was added. The mixture was washed with NaHCO₃ solution (saturated) and the organic layer was washed with brine, dried (MgSO₄) and the solvent was evaporated under reduced pressure. The residue was purified by chromatography on silica gel, eluting with CH₂Cl₂/MeOH/NH₃ (Aq.) (99:1:0.1) to give the title compound as a colorless oil (1 g).

### Description 81: Phenylmethyl 4-(2-Fluoro-1,1-dimethylethyl)-1-piperazinecarboxylate

Phenylmethyl 4-(2-hydroxy-1,1-dimethylethyl)-1-piperazinecarboxylate (Description 80, 800 mg, 2.74 mmol) in CH₂Cl₂ (25 mL) was added to a stirred, cooled (−78° C.) solution of (N-ethylethanaminato)trifluorosulfur (0.542 mL, 4.10 mmol) in CH₂Cl₂ (25 mL) and the mixture was stirred at −78° C. for 30 min. The mixture was allowed to warm to RT, diluted with CH₂Cl₂ (25 mL), washed with water (50 mL) and NaOH (4M), dried (MgSO₄), and the solvent was evaporated under reduced pressure. The residue was purified by column chromatography on silica gel, eluting with CH₂Cl₂/MeOH/NH₃ (Aq.) (99:1:0.1) to give the title compound as a yellow oil (492 mg, 61%).

### Description 82: 4-(2-Fluoro-1,1-dimethylethyl)-1-piperazine Hydrochloride

Palladium on carbon (5%, 500 mg, 4.70 mmol) and formic acid (1 mL, 26.1 mmol) were added to a solution of phenylmethyl 4-(2-fluoro-1,1-dimethylethyl)-1-piperazinecarboxylate (Description 81, 492 mg, 1.671 mmol) in methanol (25 mL) and the mixture was stirred at RT for 30 min. The mixture was filtered through celite, washing with methanol (25 mL). Ethereal HCl (1M, 3 mL) was added and the solvent was evaporated under reduced pressure to give the title compound (320 mg, 97%). m/z (ES⁺) 161 [M+H⁺].

### Description 83: 1-[(2-Methyl-4-thiazolyl)methyl]piperazine

4-(Chloromethyl)-2-methylthiazole hydrochloride (92 g, 0.5 mol) in methanol (400 mL) was added slowly to a stirred solution of piperazine (500 g, 5.81 mol) in methanol (800 mL) and the mixture was heated under reflux for 3 h. The mixture was cooled and methanolic KOH (1 mol in 400 mL) was added. The mixture was stirred at RT overnight, filtered and the solvent was evaporated under reduced pressure. The residue was dissolved in iso-propanol, filtered and purified by flash column chromatography on silica gel, eluting with iso-propanol-triethylamine (5:1) and the residue was vacuum distilled. A fraction with b.p. 123-127° C./˜1 torr was collected to give the title compound as a light-yellow dense oil which formed hygroscopic low melting (30° C.) crystals on standing (57.1 g, 58%). ¹H NMR (D₆-DMSO) δ 7.20 (1H, s), 3.45 (2H, s), 3.15 (1H, br s), 2.60 (4H, t), 2.50 (3H, s), 2.30 (4H, m).

### Description 84: 2-Bromo-N-(3-fluorophenyl)acetamide

Bromoacetyl bromide (23.5 mL, 0.27 mol) was added slowly to a stirred, cooled mixture of 3-fluoroaniline (25 g, 0.225 mol) in EtOAc (250 mL) and aqueous KHCO₃ solution (20%, 250 mL). The mixture was allowed to warm to RT and the layers were separated. The organic layer was diluted with EtOAc, washed with aqueous citric acid (10%) and brine, dried (MgSO₄) and the solvent was evaporated under reduced pressure to give the title compound (51.72 g, 92%), ¹H NMR (360 MHz, CD₃OD) δ 7.54 (1H, dt, J 11, 2), 7.34-7.24 (2H, m), 6.84 (1H, t, J 7), 3.96 (2H, s).

### Description 85: 1-(3-Fluorophenyl)piperazinone Hydrochloride

Ethanolamine (4.5 mL, 75 mmol) was added to stirred 2-bromo-N-(3-fluorophenyl)acetamide (Description 84, 5.0 g, 21 mmol) in EtOAc (25 mL) and the mixture was stirred at 60° C. for 2 h. The mixture was cooled, EtOAc and water were added and the layers were separated. The organic layer was dried (MgSO₄) and the solvent was evaporated under reduced pressure. The residue was suspended in EtOAc (135 mL) and tributylphosphine (5.3 mL, 21 mmol) was added. The mixture was cooled to 0° C. and di-tert-butylazodicarboxylate (4.8 g, 21 mmol) was added dropwise over 1 h. The mixture was stirred at 0° C. for 0.5 h., then at 40° C. for 1.5 h. The mixture was cooled and ethanolic HCl (4M, 4.7 mL) was added. The mixture was stored at 0° C. for 0.5 h., and the solid was collected, washing with cold EtOAc, and dried in vacuo to give the title compound. ¹H NMR (400 MHz, CD₃OD) δ 3.68-3.71 (2H, m), 3.97-4.00 (2H, m), 4.02 (2H, s), 7.12 (1H, m), 7.18-7.22 (2H, m), 7.48 (1H, m).

### Description 86: (RS)-1,4-Bis(phenylmethyl)-2-piperazinemethanol

Lithium aluminium hydride (1M in THF, 50.0 mL, 50.0 mmol) was added dropwise to a stirred, cooled (0° C.) solution of (RS)-ethyl 1,4-bis(phenylmethyl)-2-piperazinecarboxylate (8.65 g, 25.5 mmol) in THF (100 mL) and the mixture was stirred at RT for 17 h. The mixture was cooled to 0° C. and water (2 mL), then aqueous NaOH (4M, 8 mL) and water (2 mL) were added slowly. The mixture was stirred at RT for 1 h., filtered through Celite®, washing with THF (100 mL), and the solvent was evaporated under reduced pressure to give the title compound as a colorless oil (6.8 g, 90%). m/z (ES⁺) 296 [M+H⁺]

### Description 87: (RS)-1,1-Dimethylethyl[1,4-Bis(phenylmethyl)-2-piperazinemethoxy]acetate

1,1-Dimethylethyl bromoacetate (20 mL, 140 mmol) was added to a rapidly stirred mixture of (RS)-1,4-bis(phenylmethyl)-2-piperazinemethanol (Description 86, 4.1 g, 13.98 mmol) and tetrabutylammonium sulfate (1.0 g, 2.96 mmol) in toluene (30 mL) and aqueous NaOH (50%, 30 mL). The mixture was stirred at RT for 4 h., then the layers were separated and the aqueous layer was extracted with toluene (2×20 mL). The combined organic fractions were dried (Na₂SO₄), and the solvent was evaporated under reduced pressure. The residue was purified by flash chromatography on silica gel, eluting with EtOAc/isohexane (5% to 20%), to give the title compound as a yellow oil (5.3 g, 93%). m/z (ES⁺) 410 [M+H⁺]

### Description 88: (RS)-Hexahydropyrazino[2,1-c][1,4]oxazin-4(3H)-one

Palladium on carbon (10%, 200 mg) was added to a solution of (RS)-1,1-dimethylethyl[1,4-bis(phenylmethyl)-2-piperazinemethoxy]acetate (Description 87, 1.3 g, 3.17 mmol) in ethanol (20 mL) and the mixture was shaken under hydrogen (45 psi) for 36 h. The mixture was filtered and the solvent was evaporated under reduced pressure. The residue was dissolved in ethanol (20 mL), sodium ethoxide (200 mg) was added and the mixture was stirred at 85° C. for 72 h. The mixture was cooled and the solvent was evaporated under reduced pressure. The residue was dissolved in methanol and applied to a Bond Elut™ cartridge (SCX, 5 g). The cartridge was washed with MeOH and then eluted with methanolic ammonia (2M, 25 mL). The solvent was under reduced pressure to give the title compound as a colorless solid (380 mg, 77%). m/z (ES⁺) 157 (M+H).

### Description 89: (RS)-Octahydropyrazino[2,1-c][1,4]oxazine

Borane-THF complex (1M in tetrahydrofuran, 30 mL, 30 mmol) was added to a stirred, cooled (0° C.) solution of (RS)-hexahydropyrazino[2,1-c][1,4]oxazin-4(3H)-one (Description 88, 1.6 g, 10.2 mmol) in THF (30 mL) and the mixture was stirred at 60° C. for 24 h. The mixture was cooled to 0° C., methanol was added slowly and the mixture was stirred at RT for 2 h. The solvent was evaporated under reduced pressure and the residue was dissolved in methanol and applied to a Bond Elut™ cartridge (SCX, 10 g). The cartridge was washed with MeOH and then eluted with methanolic ammonia (2M, 50 mL). The solvent was under reduced pressure to give the title compound as a colorless solid (1.0 g, 67%). m/z (ES⁺) 143 (M+H).

### Description 90: 2,2-Dimethyl-N-pyrazin-2-ylpropanamide

Pivaloyl chloride (6.46 mL, 53 mmol) was added dropwise to a stirred, cooled (0° C.) mixture of pyrazinamine (5.0 g, 53 mmol) and triethylamine (8.06 mL, 58 mmol) in CH₂Cl₂ (80 mL) and the red mixture was stirred at 0° C. for 1 h., then at RT for 2 h. The mixture was filtered, washing with CH₂Cl₂ and the solvent was evaporated under reduced pressure. The residue was purified by flash chromatography on silica gel eluting with CH₂Cl₂/methanol 95:5 to give the title compound (8.16 g, 87%). m/z (ES⁺) 180 [M+H⁺].

### Description 91: N′-Hydroxy-2,2-dimethyl-N-pyrazin-2-ylpropanimidamide

Phosphorous pentachloride (12.45 g, 59 mmol) was added to a solution of 2,2-dimethyl-N-pyrazin-2-ylpropanamide (Description 90, 8.16 g, 46 mmol) in 1,2-dichloroethane (200 mL) and the mixture was heated under reflux for 11 h. The mixture was cooled and the solvent was evaporated under reduced pressure. The residue was suspended in THF (150 mL) and aqueous hydroxylamine (50%, 18 mL) was added dropwise. The mixture was stirred at RT overnight then aqueous NaHCO₃ was added and the mixture was extracted with EtOAc (3×). The combined organic fractions were dried (MgSO₄) and the solvent was evaporated under reduced pressure to give the title compound (8.76, 98%). m/z (ES⁺) 195 [M+H⁺].

### Description 92: 2-(1,1-Dimethylethyl)-[1,2,4]triazolo[1,5-a]pyrazine

A mixture of N′-hydroxy-2,2-dimethyl-N-pyrazin-2-ylpropanimidamide (Description 91, 8.76 g, 46 mmol) and polyphosphoric acid (90 mL) was stirred at 130° C. overnight, cooled and added to ice. The mixture was neutralized with aqueous ammonium hydroxide and extracted with EtOAc (3×). The combined organic fractions were dried (MgSO₄) and the solvent was evaporated under reduced pressure. The residue was purified by flash chromatography on silica gel eluting with isohexane/EtOAc (70:30) to give the title compound (1.04 g, 13%). m/z (ES⁺) 177 [M+H⁺].

### Description 93: 2-(1,1-Dimethylethyl)-5,6,7,8-tetrahydro[1,2,4]triazolo[1,5-a]pyrazine

Palladium on carbon (10%) was added to a solution of 2-(1,1-dimethylethyl)-[1,2,4]triazolo[1,5-a]pyrazine (Description 92, 0.50 g, 2.83 mmol) in ethanol (15 mL) and the mixture was stirred under hydrogen (1 atmosphere) overnight. The mixture was filtered, washing with ethanol, and the solvent was evaporated under reduced pressure to give the title compound (0.47 g, 92%). m/z (ES⁺) 181 [M+H⁺].

### Description 94: N-Pyrazin-2-ylacetamide

Prepared from pyrazineamine and acetic anhydride according to the method of Description 90. m/z (ES⁺) 138 [M+H⁺].

### Description 95: N′-Hydroxy-N-pyrazin-2-ylethanimidamide

Prepared from N-pyrazin-2-ylacetamide (Description 94) according to the method of Description 91.

### Description 96: 2-Methyl[1,2,4]triazolo[1,5-a]pyrazine

Prepared from N′-hydroxy-N-pyrazin-2-ylethanimidamide (Description 95) according to the method of Description 92. m/z (ES⁺) 135 [M+H⁺].

### Description 97: 2-Methyl-5,6,7,8-tetrahydro[1,2,4]triazolo[1,5-a]pyrazine

Prepared from 2-methyl[1,2,4]triazolo[1,5-a]pyrazine (Description 96) according to the method of Description 93. m/z (ES⁺) 139 [M+H⁺].

### Description 98: 2,3,5,6,7,8-Hexahydro-1,2,4-triazolo[4,3-a]pyrazine-3-one

Palladium on carbon (10%, 0.05 g) 1 was added to a solution of 1,2,4-triazolo[4,3-a]pyrazin-3(2H)-one (0.12 g) in absolute ethanol (50 mL) and the mixture was shaken under hydrogen (50 psi) for 16 h. The mixture was filtered and the solvent was evaporated under reduced pressure to give the title compound as a colorless solid (0.112 g). ¹H NMR (500 MHz, DMSO-d6): δ 11.42 (1H, br s), 3.72 (2H, s), 3.48 (2H, s), 3.03 (2H, s).

### EXAMPLE 103

### Methyl 2-({3-[(3-Oxo-2,3,5,6,7,8-hexahydro[1,2,4]triazolo[4,3-a]pyridin-7-yl)methyl]-2-phenylquinolin-4-yl}carbonyl)-1-phenylhydrazinecarboxylate

A mixture of methyl 2-{[3-(bromomethyl)-2-phenylquinolin-4-yl]carbonyl}-1-phenylhydrazinecarboxylate (Description 16, 0.21 g, 0.43 mmol), 2,3,5,6,7,8-hexahydro-1,2,4-triazolo[4,3-a]pyrazine-3-one (Description 98, 50 mg, 0.36 mmol) and triethylamine (0.06 mL, 44 mg, 0.43 mmol) in THF (2 mL) was heated under reflux for 16 h. The mixture was cooled and EtOAc (30 mL), water (30 mL) and aqueous NaHCO₃ (saturated, 20 mL) were added. The layers were separated and the organic layer was washed with aqueous NaHCO₃ (saturated, 20 mL) and brine (20 mL), dried (Na₂SO₄) and the solvent was evaporated under reduced pressure. The residue was purified by preparative thin layer chromatography on silica gel, eluting with EtOAc, to give the title compound as a colorless solid (87 mg, 44%). m/z (ES⁺) 550 [M+H⁺].

The following compounds were prepared according to the method of Example 103, substituting a suitable 3-(bromomethyl)-2-phenylquinoline for methyl 2-{[3-(bromomethyl)-2-phenylquinolin-4-yl]carbonyl}-1-phenylhydrazinecarboxylate and a suitable amine for 1,2,4-triazolo[4,3-a]pyrazin-3(2H)-one.

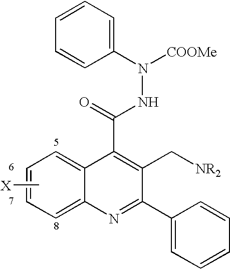

| | | | | | m/z | |
|---|---|---|---|---|---|---|
| Ex. | X | NR₂ | Formula | MWT | (ES⁺) | Name |
| 104 | 8-F | 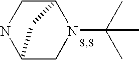 | C₃₄H₃₆FN₅O₃ | 581.7 | 582 | methyl 2-[(3-{[(1S,4S)-5-(1,1-dimethylethyl)-2,5-diazabicyclo[2.2.1]hept-2-yl]methyl}-8-fluoro-2-phenylquinolin-4-yl)carbonyl]-1-phenylhydrazinecarboxylate |
| 105 | 8-F | 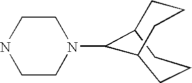 | C₃₈H₄₃FN₅O₃ | 635.79 | 636 | methyl 2-({3-[(4-bicyclo[3.3.1]non-9-ylpiperazin-1-yl)methyl]-8-fluoro-2-phenylquinolin-4-yl}carbonyl)-1-phenylhydrazinecarboxylate |
| 106 | 8-F | 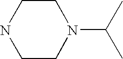 | C₃₂H₃₅FN₅O₃ | 555.66 | 556 | methyl 2-({8-fluoro-3-[(4-isopropylpiperazin-1-yl)methyl]-2-phenylquinolin-4-yl}carbonyl)-1-phenylhydrazinecarboxylate |
| 107 | 8-F | 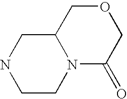 | C₃₂H₃₁FN₅O₅ | 583.63 | 584 | methyl 2-({8-fluoro-3-[(4-oxohexahydropyrazino[2,1-c][1,4]oxazin-8(1H)-yl)methyl]-2-phenylquinolin-4-yl}carbonyl)-1-phenylhydrazinecarboxylate |
| 108 | 8-F | 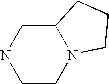 | C₃₂H₃₃FN₅O₃ | 553.64 | 554 | methyl 2-{[8-fluoro-3-(hexahydropyrrolo[1,2-a]pyrazin-2(1H)-ylmethyl)-2-phenylquinolin-4-yl]carbonyl}-1-phenylhydrazinecarboxylate |
| 109 | 8-F | 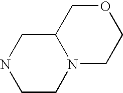 | C₃₂H₃₃FN₅O₄ | 569.64 | 570 | methyl 2-{[8-fluoro-3-(hexahydropyrazino[2,1-c][1,4]oxazin-8(1H)-ylmethyl)-2-phenylquinolin-4-yl]carbonyl}-1-phenylhydrazinecarboxylate |
| 110 | 8-F | 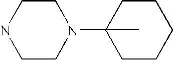 | C₃₆H₄₁FN₅O₃ | 609.75 | 610 | methyl 2-[(8-fluoro-3-{[4-(1-methylcyclohexyl)piperazin-1-yl]methyl}-2-phenylquinolin-4-yl)carbonyl]-1-phenylhydrazinecarboxylate |
| 111 | H | 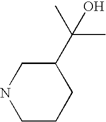 | C₃₃H₃₆N₄O₄ | 552.68 | 553 | methyl 2-[(3-{[3-(1-hydroxy-1-methylethyl)piperidin-1-yl]methyl}-2-phenylquinolin-4-yl)carbonyl]-1-phenylhydrazinecarboxylate |
| 112 | H | 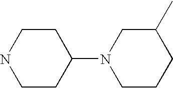 | C₃₆H₄₁N₅O₃ | 591.76 | 592 | methyl 2-({3-[(3-methyl-1,4′-bipiperidin-1′-yl)methyl]-2-phenylquinolin-4-yl}carbonyl)-1-phenylhydrazinecarboxylate |
| 113 | H | 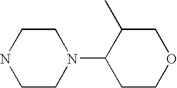 | C₃₅H₃₉N₅O₄ | 593.73 | 594 | methyl 2-[(3-{[4-(3-methyltetrahydro-2H-pyran-4-yl)piperazin-1-yl]methyl}-2-phenylquinolin-4-yl)carbonyl]-1-phenylhydrazinecarboxylate |
| 114 | H | 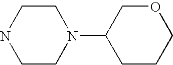 | C₃₄H₃₇N₅O₄ | 579.71 | 580 | methyl 1-phenyl-2-[(2-phenyl-3-{[4-(tetrahydro-2H-pyran-3-yl)piperazin-1-yl]methyl}quinolin-4-yl)carbonyl]hydrazinecarboxylate |
| 115 | H | 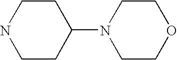 | C₃₄H₃₇N₅O₄ | 579.71 | 580 | methyl 2-({3-[(4-morpholin-4-ylpiperidin-1-yl)methyl]-2-phenylquinolin-4-yl}carbonyl)-1-phenylhydrazinecarboxylate |
| 116 | H | 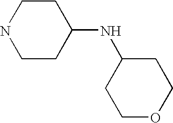 | C₃₅H₃₉N₅O₄ | 593.73 | 594 | methyl 1-phenyl-2-[(2-phenyl-3-{[4-(tetrahydro-2H-pyran-4-ylamino)piperidin-1-yl]methyl}quinolin-4-yl)carbonyl]hydrazinecarboxylate |
| 117 | H | 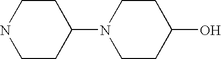 | C₃₅H₃₉N₅O₄ | 593.73 | 594 | methyl 2-({3-[(4-hydroxy-1,4′-bipiperidin-1′-yl)methyl]-2-phenylquinolin-4-yl}carbonyl)-1-phenylhydrazinecarboxylate |
| 118 | H | 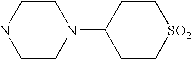 | C₃₄H₃₇N₅O₅S | 627.77 | 628 | methyl 2-[(3-{[4-(1,1-dioxidotetrahydro-2H-thiopyran-4-yl)piperazin-1-yl]methyl}-2-phenylquinolin-4-yl)carbonyl]-1-phenylhydrazinecarboxylate |
| 119 | H | 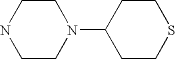 | C₃₄H₃₇N₅O₃S | 595.77 | 596 | methyl 1-phenyl-2-[(2-phenyl-3-{[4-(tetrahydro-2H-thiopyran-4-yl)piperazin-1-yl]methyl}quinolin-4-yl)carbonyl]hydrazinecarboxylate |
| 120 | H | 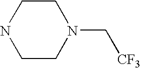 | C₃₁H₃₀F₃N₅O₃ | 577.61 | 578 | methyl 1-phenyl-2-[(2-phenyl-3-{[4-(2,2,2-trifluoroethyl)piperazin-1-yl]methyl}quinolin-4-yl)carbonyl]hydrazinecarboxylate |
| 121 | H | 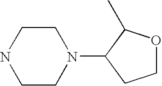 | C₃₄H₃₈N₅O₄ | 579.71 | 580 | 2,5-anhydro-1,3,4-trideoxy-3-{4-[(4-{[2-(methoxycarbonyl)-2-phenylhydrazino]carbonyl}-2-phenylquinolin-3-yl)methyl]piperazin-1-yl}pentitol |
| 122 | 8-F | 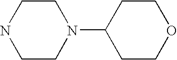 | C₃₄H₃₆FN₅O₄ | 597.7 | 598 | methyl 2-[(8-fluoro-2-phenyl-3-{[4-(tetrahydro-2H-pyran-4-yl)piperazin-1-yl]methyl}quinolin-4-yl)carbonyl]-1-phenylhydrazinecarboxylate |
| 123 | 8-F | 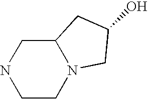 | C₃₂H₃₃FN₅O₄ | 569.64 | 570 | methyl 2-[(8-fluoro-3-{[7S)-7-hydroxyhexahydropyrrolo[1,2-a]pyrazin-2(1H)-yl]methyl}-2-phenylquinolin-4-yl)carbonyl]-1-phenylhydrazinecarboxylate |
| 124 | 8-F | 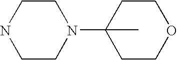 | C₃₅H₃₈FN₅O₄ | 611.72 | 612 | methyl 2-[(8-fluoro-3-{[4-(4-methyltetrahydro-2H-pyran-4-yl)piperazin-1-yl]methyl}-2-phenylquinolin-4-yl)carbonyl]-1-phenylhydrazinecarboxylate |
| 125 | H | 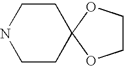 | C₃₂H₃₂N₄O₅ | 552.64 | 553 | methyl 2-{[3-(1,4-dioxa-8-azaspiro[4.5]dec-8-ylmethyl)-2-phenylquinolin-4-yl]carbonyl}-1-phenylhydrazinecarboxylate |
| 126 | H | 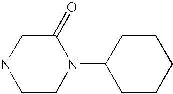 | C₃₅H₃₇N₅O₄ | 591.72 | 592 | methyl 2-({3-[(4-cyclohexyl-3-oxopiperazin-1-yl)methyl]-2-phenylquinolin-4-yl}carbonyl)-1-phenylhydrazinecarboxylate |
| 127 | H | 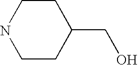 | C₃₁H₃₂N₄O₄ | 524.63 | 525 | methyl 2-[(3-{[4-(hydroxymethyl)piperidin-1-yl]methyl}-2-phenylquinolin-4-yl)carbonyl]-1-phenylhydrazinecarboxylate |
| 128 | H | 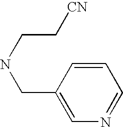 | C₃₄H₃₀N₆O₃ | 570.66 | 571 | methyl 2-[(3-{[(2-cyanoethyl)(pyridin-3-ylmethyl)amino]methyl}-2-phenylquinolin-4-yl)carbonyl]-1-phenylhydrazinecarboxylate |
| 129 | H | 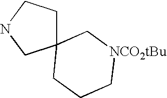 | C₃₈H₄₃N₅O₅ | 649.8 | 650 | 1,1-dimethylethyl 2-[(4-{[2-(methoxycarbonyl)-2-phenylhydrazino]carbonyl}-2-phenylquinolin-3-yl)methyl]-1,7-diazaspiro[4.5]decane-7-carboxylate |
| 130 | H | 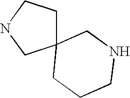 | C₃₃H₃₅N₅O₃ | 549.68 | 550 | methyl 2-{[3-(2,7-diazaspiro[4.5]dec-2-ylmethyl)-2-phenylquinolin-4-yl]carbonyl}-1-phenylhydrazinecarboxylate |
| 131 | H | 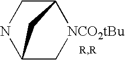 | C₃₅H₃₇N₅O₅ | 607.72 | 608 | 1,1-dimethylethyl (1R,4R)-5-[(4-{[2-(methoxycarbonyl)-2-phenylhydrazino]carbonyl}-2-phenylquinolin-3-yl)methyl]-2,5-diazabicyclo[2.2.1]heptane-2-carboxylate |
| 132 | H | 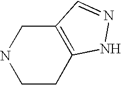 | C₃₁H₂₈N₆O₃ | 532.61 | 533 | methyl 1-phenyl-2-{[2-phenyl-3-(1,4,6,7-tetrahydro-5H-pyrazolo[4,3-c]pyridin-5-ylmethyl)quinolin-4-yl]carbonyl}hydrazinecarboxylate |
| 133 | H | 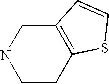 | C₃₂H₂₈N₄O₃S | 548.67 | 549 | methyl 2-{[3-(6,7-dihydrothieno[3,2-c]pyridin-5(4H)-ylmethyl)-2-phenylquinolin-4-yl]carbonyl}-1-phenylhydrazinecarboxylate |
| 134 | H | 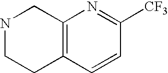 | C₃₄H₂₈F₃N₅O₃ | 611.63 | 612 | methyl 1-phenyl-2-[(2-phenyl-3-{[2-(trifluoromethyl)-5,8-dihydro-1,7-naphthyridin-7(6H)-yl]methyl}quinolin-4-yl)carbonyl]hydrazinecarboxylate |
| 135 | H | 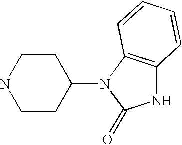 | C₃₇H₃₄N₆O₄ | 626.72 | 627 | methyl 2-[(3-{[4-(2-oxo-2,3-dihydro-1H-benzimidazol-1-yl)piperidin-1-yl]methyl}-2-phenylquinolin-4-yl)carbonyl]-1-phenylhydrazinecarboxylate |
| 136 | H | 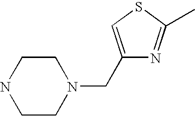 | C₃₄H₃₄N₆O₃S | 606.75 | 607 | methyl 2-{[3-({4-[(2-methyl-1,3-thiazol-4-yl)methyl]piperazin-1-yl}methyl)-2-phenylquinolin-4-yl]carbonyl}-1-phenylhydrazinecarboxylate |
| 137 | H | 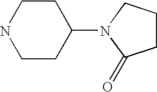 | C₃₄H₃₅N₅O₄ | 577.69 | 578 | methyl 2-[(3-{[4-(2-oxopyrrolidin-1-yl)piperidin-1-yl]methyl}-2-phenylquinolin-4-yl)carbonyl]-1-phenylhydrazinecarboxylate |
| 138 | H | 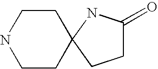 | C₃₃H₃₂N₄O₅ | 564.65 | 565 | methyl 2-({3-[(2-oxo-1-oxa-8-azaspiro[4.5]dec-8-yl)methyl]-2-phenylquinolin-4-yl}carbonyl)-1-phenylhydrazinecarboxylate |
| 139 | H | 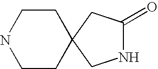 | C₃₃H₃₃N₅O₄ | 563.66 | 564 | methyl 2-({3-[(3-oxo-2,8-diazaspiro[4.5]dec-8-yl)methyl]-2-phenylquinolin-4-yl}carbonyl)-1-phenylhydrazinecarboxylate |
| 140 | H | 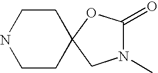 | C₃₃H₃₃N₅O₅ | 579.66 | 580 | methyl 2-({3-[(3-methyl-2-oxo-1-oxa-3,8-diazaspiro[4.5]dec-8-yl)methyl]-2-phenylquinolin-4-yl}carbonyl)-1-phenylhydrazinecarboxylate |
| 141 | H | 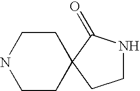 | C₃₃H₃₃N₅O₄ | 563.66 | 564 | methyl 2-({3-[(3-oxo-2,8-diazaspiro[4.5]dec-8-yl)methyl]-2-phenylquinolin-4-yl}carbonyl)-1-phenylhydrazinecarboxylate |
| 142 | H | 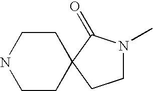 | C₃₄H₃₅N₅O₄ | 577.69 | 578 | methyl 2-({3-[(2-methyl-1-oxo-2,8-diazaspiro[4.5]dec-8-yl)methyl]-2-phenylquinolin-4-yl}carbonyl)-1-phenylhydrazinecarboxylate |
| 143 | H | 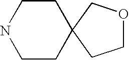 | C₃₃H₃₄N₄O₄ | 550.66 | 551 | methyl 2-{[3-(2-oxa-8-azaspiro[4.5]dec-8-ylmethyl)-2-phenylquinolin-4-yl]carbonyl}-1-phenylhydrazinecarboxylate |
| 144 | H | 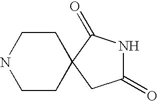 | C₃₃H₃₁N₅O₅ | 577.65 | 578 | methyl 2-({3-[(1,3-dioxo-2,8-diazaspiro[4.5]dec-8-yl)methyl]-2-phenylquinolin-4-yl}carbonyl)-1-phenylhydrazinecarboxylate |
| 145 | H | 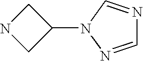 | C₃₀H₂₇N₇O₃ | 533.6 | 534 | methyl 1-phenyl-2-[(2-phenyl-3-{[3-(1H-1,2,4-triazol-1-yl)azetidin-1-yl]methyl}quinolin-4-yl)carbonyl]hydrazinecarboxylate |
| 146 | H | 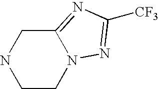 | C₃₁H₂₆F₃N₇O₃ | 601.59 | 602 | methyl 1-phenyl-2-[(2-phenyl-3-{[2-(trifluoromethyl)-5,6-dihydro[1,2,4]triazolo[1,5-a]pyrazin-7(8H)-yl]methyl}quinolin-4-yl)carbonyl]hydrazinecarboxylate |
| 147 | H | 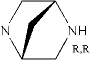 | C₃₀H₂₉N₅O₃ | 507.6 | 508 | methyl 2-({3-[(1R,4R)-2,5-diazabicyclo[2.2.1]hept-2-ylmethyl]-2-phenylquinolin-4-yl}carbonyl)-1-phenylhydrazinecarboxylate |
| 148 | H | 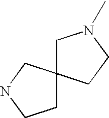 | C₃₃H₃₅N₅O₃ | 549.68 | 550 | methyl 2-({3-[(1-methyl-1,7-diazaspiro[4.4]non-7-yl)methyl]-2-phenylquinolin-4-yl}carbonyl)-1-phenylhydrazinecarboxylate |
| 149 | H | 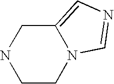 | C₃₁H₂₈N₆O₃ | 532.61 | 533 | methyl 2-({3-[(1-methyl-1,7-diazaspiro[4.4]non-7-yl)methyl]-2-phenylquinolin-4-yl}carbonyl)-1-phenylhydrazinecarboxylate |
| 150 | H | 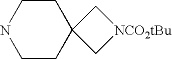 | C₃₇H₄₁N₅O₅ | 635.77 | 636 | 1,1-dimethylethyl 7-[(4-{[2-(methoxycarbonyl)-2-phenylhydrazino]carbonyl}-2-phenylquinolin-3-yl)methyl]-2,7-diazaspiro[3.5]nonane-2-carboxylate |
| 151 | H | 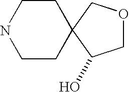 | C₃₃H₃₄N₄O₅ | 566.66 | 567 | methyl 2-[(3-{[(4R)-4-hydroxy-2-oxa-8-azaspiro[4.5]dec-8-yl]methyl}-2-phenylquinolin-4-yl)carbonyl]-1-phenylhydrazinecarboxylate |
| 152 | H | 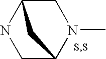 | C₃₁H₃₁N₅O₃ | 521.62 | 522 | methyl 2-[(3-{[(1S,4S)-5-methyl-2,5-diazabicyclo[2.2.1]hept-2-yl]methyl}-2-phenylquinolin-4-yl)carbonyl]-1-phenylhydrazinecarboxylate |
| 153 | H | 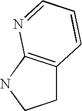 | C₃₂H₂₇N₅O₃ | 529.6 | 530 | methyl 2-{[3-(2,3-dihydro-1H-pyrrolo[2,3-b]pyridin-1-ylmethyl)-2-phenylquinolin-4-yl]carbonyl}-1-phenylhydrazinecarboxylate |
| 154 | H | 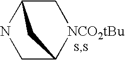 | C₃₅H₃₇N₅O₅ | 607.72 | 608 | 1,1-dimethylethyl (1S,4S)-5-[(4-{[2-(methoxycarbonyl)-2-phenylhydrazino]carbonyl}-2-phenylquinolin-3-yl)methyl]-2,5-diazabicyclo[2.2.1]heptane-2-carboxylate |
| 155 | H | 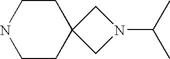 | C₃₅H₃₉N₅O₃ | 577.73 | 578 | methyl 2-({3-[(2-isopropyl-2,7-diazaspiro[3.5]non-7-yl)methyl]-2-phenylquinolin-4-yl}carbonyl)-1-phenylhydrazinecarboxylate |
| 156 | H | 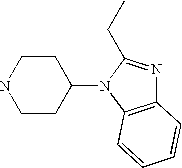 | C₃₉H₃₈N₆O₃ | 638.78 | 639 | methyl 2-[(3-{[4-(2-ethyl-1H-benzimidazol-1-yl)piperidin-1-yl]methyl}-2-phenylquinolin-4-yl)carbonyl]-1-phenylhydrazinecarboxylate |
| 157 | H | 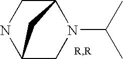 | C₃₃H₃₅N₅O₃ | 549.68 | 550 | methyl 2-[(3-{[(1R,4R)-5-isopropyl-2,5-diazabicyclo[2.2.1]hept-2-yl]methyl}-2-phenylquinolin-4-yl)carbonyl]-1-phenylhydrazinecarboxylate |
| 158 | H | 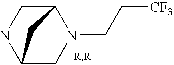 | C₃₃H₃₂F₃N₅O₃ | 603.65 | 604 | methyl 1-phenyl-2-[(2-phenyl-3-{[(1R,4R)-5-(3,3,3-trifluoropropyl)-2,5-diazabicyclo[2.2.1]hept-2-yl]methyl}quinolin-4-yl)carbonyl]hydrazinecarboxylate |
| 159 | H | 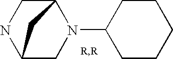 | C₃₆H₃₉N₅O₃ | 589.74 | 590 | methyl 2-[(3-{[(1R,4R)-5-cyclohexyl-2,5-diazabicyclo[2.2.1]hept-2-yl]methyl}-2-phenylquinolin-4-yl)carbonyl]-1-phenylhydrazinecarboxylate |
| 160 | H | 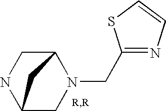 | C₃₄H₃₂N₆O₃S | 604.74 | 605 | methyl 1-phenyl-2-[(2-phenyl-3-{[(1R,4R)-5-(1,3-thiazol-2-ylmethyl)-2,5-diazabicyclo[2.2.1]hept-2-yl]methyl}quinolin-4-yl)carbonyl]hydrazinecarboxylate |
| 161 | H | 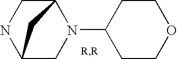 | C₃₅H₃₇N₅O₄ | 591.72 | 592 | methyl 1-phenyl-2-[(2-phenyl-3-{[(1R,4R)-5-(tetrahydro-2H-pyran-4-yl)-2,5-diazabicyclo[2.2.1]hept-2-yl]methyl}quinolin-4-yl)carbonyl]hydrazinecarboxylate |
| 162 | H | 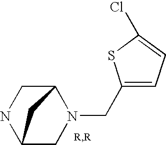 | C₃₅H₃₂C₁N₅O₃S | 638.19 | 638/640 | methyl 2-{[3-({(1R,4R)-5-[(5-chloro-2-thienyl)methyl]-2,5-diazabicyclo[2.2.1]hept-2-yl}methyl)-2-phenylquinolin-4-yl]carbonyl}-1-phenylhydrazinecarboxylate |
| 163 | H | 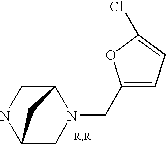 | C₃₅H₃₂C₁N₅O₄ | 622.13 | 622/624 | methyl 2-{[3-({(1R,4R)-5-[(5-chloro-2-furyl)methyl]-2,5-diazabicyclo[2.2.1]hept-2-yl}methyl)-2-phenylquinolin-4-yl]carbonyl}-1-phenylhydrazinecarboxylate |
| 164 | H | 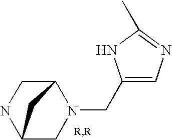 | C₃₅H₃₅N₇O₃ | 601.71 | 602 | methyl 2-{[3-({(1R,4R)-5-[(2-methyl-1H-imidazol-5-yl)methyl]-2,5-diazabicyclo[2.2.1]hept-2-yl}methyl)-2-phenylquinolin-4-yl]carbonyl}-1-phenylhydrazinecarboxylate |
| 165 | H | 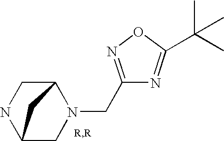 | C₃₇H₃₉N₇O₄ | 645.77 | 646 | methyl 2-({3-[((1R,4R)-5-{[5-(1,1-dimethylethyl)-1,2,4-oxadiazol-3-yl]methyl}-2,5-diazabicyclo[2.2.1]hept-2-yl)methyl]-2-phenylquinolin-4-yl}carbonyl)-1-phenylhydrazinecarboxylate |
| 166 | H | 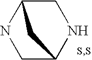 | C₃₀H₂₉N₅O₃ | 507.6 | 508 | methyl 2-({3-[(1R,4S)-2,5-diazabicyclo[2.2.1]hept-2-ylmethyl]-2-phenylquinolin-4-yl}carbonyl)-1-phenylhydrazinecarboxylate |
| 167 | H | 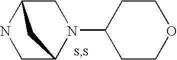 | C₃₅H₃₇N₅O₄ | 591.72 | 592 | methyl 1-phenyl-2-[(2-phenyl-3-{[(1S,4S)-5-(tetrahydro-2H-pyran-4-yl)-2,5-diazabicyclo[2.2.1]hept-2-yl]methyl}quinolin-4-yl)carbonyl]hydrazinecarboxylate |
| 168 | H | 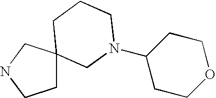 | C₃₈H₄₃N₅O₄ | 633.8 | 634 | methyl 1-phenyl-2-[(2-phenyl-3-{[7-(tetrahydro-2H-pyran-4-yl)-2,7-diazaspiro[4.5]dec-2-yl]methyl}quinolin-4-yl)carbonyl]hydrazinecarboxylate |
| 169 | H | 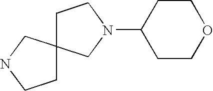 | C₃₇H₄₁N₅O₄ | 619.77 | 620 | methyl 1-phenyl-2-[(2-phenyl-3-{[7-(tetrahydro-2H-pyran-4-yl)-2,7-diazaspiro[4.4]non-2-yl]methyl}quinolin-4-yl)carbonyl]hydrazinecarboxylate |
| 170 | H | 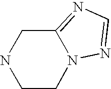 | C₃₀H₂₇N₇O₃ | 533.6 | 534 | methyl 2-{[3-(5,6-dihydro[1,2,4]triazolo[1,5-a]pyrazin-7(8H)-ylmethyl)-2-phenylquinolin-4-yl]carbonyl}-1-phenylhydrazinecarboxylate |
| 171 | H | 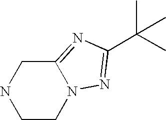 | C₃₄H₃₅N₇O₃ | 589.7 | 590 | methyl 2-[(3-{[2-(1,1-dimethylethyl)-5,6-dihydro[1,2,4]triazolo[1,5-a]pyrazin-7(8H)-yl]methyl}-2-phenylquinolin-4-yl)carbonyl]-1-phenylhydrazinecarboxylate |
| 172 | H | 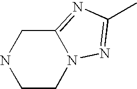 | C₃₁H₂₉N₇O₃ | 547.62 | 548 | methyl 2-({3-[(2-methyl-5,6-dihydro[1,2,4]triazolo[1,5-a]pyrazin-7(8H)-yl)methyl]-2-phenylquinolin-4-yl}carbonyl)-1-phenylhydrazinecarboxylate |
| 173 | 8-F | 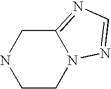 | C₃₀H₂₆FN₇O₃ | 551.59 | 552 | methyl 2-{[3-(5,6-dihydro[1,2,4]triazolo[1,5-a]pyrazin-7(8H)-ylmethyl)-8-fluoro-2-phenylquinolin-4-yl]carbonyl}-1-phenylhydrazinecarboxylate |
| 174 | H | 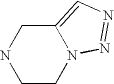 | C₃₀H₂₇N₇O₃ | 533.6 | 534 | methyl 2-{[3-(6,7-dihydro[1,2,3]triazolo[1,5-a]pyrazin-5(4H)-ylmethyl)-2-phenylquinolin-4-yl]carbonyl}-1-phenylhydrazinecarboxylate |
| 175 | 5-F | 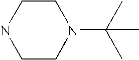 | C₃₃H₃₇FN₅O₃ | 569.69 | 570 | methyl 2-[(3-{[4-(1,1-dimethylethyl)piperazin-1-yl]methyl}-5-fluoro-2-phenylquinolin-4-yl)carbonyl]-1-phenylhydrazinecarboxylate |
| 176 | H | 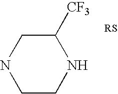 | C₃₀H₂₈F₃N₅O₃ | 563.59 | 564 | methyl 2-[(3-{[3-(trifluoromethyl)piperazin-1-yl]methyl}-2-phenylquinolin-4-yl)carbonyl]-1-phenylhydrazinecarboxylate |
| 177 | 5-F | 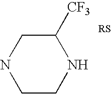 | C₃₀H₂₇F₄N₅O₃ | 581.58 | 582 | methyl 2-[(3-{[3-(trifluoromethyl)piperazin-1-yl]methyl}-5-fluoro-2-phenylquinolin-4-yl)carbonyl]-1-phenylhydrazinecarboxylate |
| 178 | H | 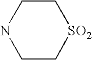 | C₂₉H₂₈N₄O₅S | 544.63 | 545 | methyl 2-({3-[(1,1-dioxidothiomorpholin-4-yl)methyl]-2-phenylquinolin-4-yl}carbonyl)-1-phenylhydrazinecarboxylate |
| 179 | H | 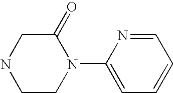 | C₃₄H₃₀N₆O₄ | 586.66 | 587 | methyl 2-({3-[(3-oxo-4-pyridin-2-ylpiperazin-1-yl)methyl]-2-phenylquinolin-4-yl}carbonyl)-1-phenylhydrazinecarboxylate |
| 180 | H | 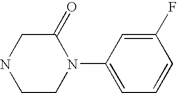 | C₃₅H₃₁FN₅O₄ | 603.66 | 604 | methyl 2-[(3-{[4-(3-fluorophenyl)-3-oxopiperazin-1-yl]methyl}-2-phenylquinolin-4-yl)carbonyl]-1-phenylhydrazinecarboxylate |
| 181 | H | 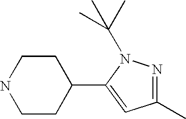 | C₃₈H₄₂N₆O₃ | 630.8 | 632 | methyl 2-{[3-({4-[1-(1,1-dimethylethyl)-3-methyl-1H-pyrazol-5-yl]piperidin-1-yl}methyl)-2-phenylquinolin-4-yl]carbonyl}-1-phenylhydrazinecarboxylate |
| 182 | H | 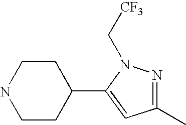 | C₃₆H₃₅F₃N₆O₃ | 656.71 | 657 | methyl 2-{[3-({4-[3-methyl-1-(2,2,2-trifluoroethyl)-1H-pyrazol-5-yl]piperidin-1-yl}methyl)-2-phenylquinolin-4-yl]carbonyl}-1-phenylhydrazinecarboxylate |
| 183 | H | 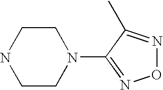 | C₃₂H₃₁N₇O₄ | 577.65 | 578 | methyl 2-[(3-{[4-(4-methyl-1,2,5-oxadiazol-3-yl)piperazin-1-yl]methyl}-2-phenylquinolin-4-yl)carbonyl]-1-phenylhydrazinecarboxylate |
| 184 | H | 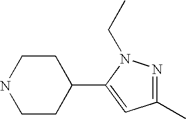 | C₃₆H₃₈N₆O₃ | 602.74 | 604 | methyl 2-[(3-{[4-(1-ethyl-3-methyl-1H-pyrazol-5-yl)piperidin-1-yl]methyl}-2-phenylquinolin-4-yl)carbonyl]-1-phenylhydrazinecarboxylate |
| 185 | H | 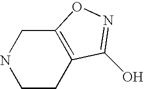 | C₃₁H₂₇N₅O₅ | 549.59 | 550 | methyl 2-({3-[(3-hydroxy-4,7-dihydroisoxazolo[5,4-c]pyridin-6(5H)-yl)methyl]-2-phenylquinolin-4-yl}carbonyl)-1-phenylhydrazinecarboxylate |
| 186 | H | 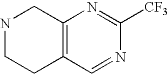 | C₃₃H₂₇F₃N₆O₃ | 612.62 | 613 | methyl 1-phenyl-2-[(2-phenyl-3-{[2-(trifluoromethyl)-5,8-dihydropyrido[3,4-d]pyrimidin-7(6H)-yl]methyl}quinolin-4-yl)carbonyl]hydrazinecarboxylate |
| 187 | H | 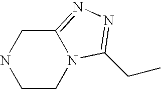 | C₃₂H₃₁N₇O₃ | 561.65 | 562 | methyl 2-({3-[(3-ethyl-5,6-dihydro[1,2,4]triazolo[4,3-a]pyrazin-7(8H)-yl)methyl]-2-phenylquinolin-4-yl}carbonyl)-1-phenylhydrazinecarboxylate-methane(1:1) |
| 188 | H | 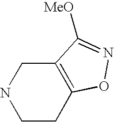 | C₃₂H₂₉N₅O₅ | 563.62 | 564 | methyl 2-({3-[(3-methoxy-6,7-dihydroisoxazolo[4,5-c]pyridin-5(4H)-yl)methyl]-2-phenylquinolin-4-yl}carbonyl)-1-phenylhydrazinecarboxylate |
| 189 | H | 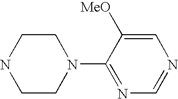 | C₃₄H₃₃N₇O₄ | 603.69 | 604 | methyl 2-[(3-{[4-(5-methoxypyrimidin-4-yl)piperazin-1-yl]methyl}-2-phenylquinolin-4-yl)carbonyl]-1-phenylhydrazinecarboxylate |
| 190 | 7-Br | 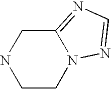 | C₃₀H₂₆BrN₇O₃ | 612.49 | 612/614 | methyl 2-{[7-bromo-3-(5,6-dihydro[1,2,4]triazolo[1,5-a]pyrazin-7(8H)-ylmethyl)-2-phenylquinolin-4-yl]carbonyl}-1-phenylhydrazinecarboxylate |
| 191 | 7-Br | 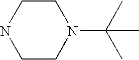 | C₃₃H₃₆BrN₅O₃ | 630.59 | 630/632 | methyl 2-[(7-bromo-3-{[4-(1,1-dimethylethyl)piperazin-1-yl]methyl}-2-phenylquinolin-4-yl)carbonyl]-1-phenylhydrazinecarboxylate |
| 192 | H | 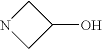 | C₂₈H₂₆N₄O₄ | 482.54 | 483 | methyl 2-{[3-(3′-hydroxyazetidnylmethyl)-2-phenylquinolin-4-yl]carbonyl}-1-phenylhydrazinecarboxylate |
| 193 | H | 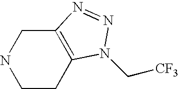 | C₃₂H₂₈F₃N₇O₃ | 615.62 | 616 | methyl 2-{[3-(-(1-N(trifluoroethyl,1,2,3,triazolopiperidinyl[4,3-a]methyl)-2-phenylquinolin-4-yl]carbonyl}-1-phenylhydrazinecarboxylate? |
| 194 | H | 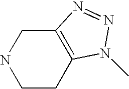 | C₃₁H₂₉N₇O₃ | 547.62 | 548 | methyl 2-{[3-(1-N(methyl,1,2,3,triazolopiperidinyl[4,3-a]methyl)-2-phenylquinolin-4-yl]carbonyl}-1-phenylhydrazinecarboxylate? |
| 195 | 8-F | 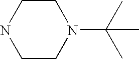 | C₃₃H₃₆FN₅O₃ | 569.69 | 570 | methyl 2-[(3-{[4-(1,1-dimethylethyl)piperazin-1-yl]methyl}-2-phenylquinolin-4-yl)carbonyl]-1-phenylhydrazinecarboxylate |
| 196 | 8-F | 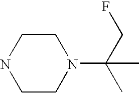 | C₃₃H₃₆F₂N₅O₃ | 587.68 | 588 | methyl 2-[(3-{[4-(2-fluoro-1,1-dimethylethyl)piperazin-1-yl]methyl}-2-phenylquinolin-4-yl)carbonyl]-1-phenylhydrazinecarboxylate |

### EXAMPLE 197

### Methyl 2-[(7-Cyano-3-{[4-(1,1-dimethylethyl)piperazin-1-yl]methyl}-2-phenylquinolin-4-yl)carbonyl]-1-phenylhydrazinecarboxylate

A mixture of methyl 2-[(7-bromo-3-{[4-(1,1-dimethylethyl)piperazin-1-yl]methyl}-2-phenylquinolin-4-yl)carbonyl]-1-phenylhydrazinecarboxylate (Example 191, 104.4 mg, 0.166 mmol), zinc cyanide (11.7 mg, 0.100 mmol, zinc powder (1.7 mg, 0.026 mmol), tris(dibenzylideneacetal)palladium(0) (3.7 mg, 0.0040 mmol) and 1,1′-bis(diphenylphosphino)ferrocene (4.3 mg, 0.0078 mmol) was flushed with nitrogen, then anhydrous N,N-dimethylacetamide (2 mL) was added. The mixture was degassed with bubbling nitrogen for 10 min, then stirred vigorously at 120° C. for 75 min. The mixture was cooled, diluted with EtOAc (25 mL) and washed with aqueous ammonia (2N, 25 mL) and brine (25 mL). The organic layer was dried (Na₂SO₄) and the solvent was evaporated under reduced pressure. The residue was purified by flash column chromatography on silica gel, eluting with CH₂Cl₂/MeOH (95:5) to give the title compound as a colorless solid (92.4 mg, 97%): ¹H NMR (500 MHz, DMSO-d6) δ 11.60 (1H, s), 8.65 (1H, s), 8.06 (1H, br s), 7.54-7.44 (10H, m), 7.33 (1H, t, J 7.0), 3.82 (3H, s), 3.53 (2H, br s), 2.03 (4H, br s), 1.91 (4H, br s), 0.85 (9H, s). m/z (ES⁺) 577 [M+H⁺].

The following compounds were prepared as mixtures of diastereoisomers from methyl 1-phenyl-2-{[2-phenyl-3-(piperazin-1-ylmethyl)quinolin-4-yl]carbonyl}hydrazinecarboxylate (Example 2) and appropriately substituted cyclopentanones according to the method of Description 61.

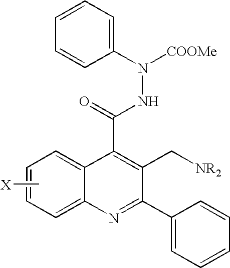

| | | | | | m/z | |
|---|---|---|---|---|---|---|
| Ex. | X | NR₂ | Formula | MWT | (ES⁺) | Name |
| 198 | H | 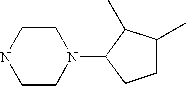 | C₃₆H₄₁N₅O₃ | 591.76 | 592 | Methyl 2-[(3-{[1-(2,3-dimethylcyclopentyl)piperazin-4-yl]methyl}-2-phenylquinolin-4-yl)carbonyl]-1-phenylhydrazinecarboxylate |
| 199 | H | 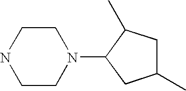 | C₃₆H₄₁N₅O₃ | 591.76 | 592 | Methyl 2-[(3-{[1-(2,4-dimethylcyclopentyl)piperazin-4-yl]methyl}-2-phenylquinolin-4-yl)carbonyl]-1-phenylhydrazinecarboxylate |
| 200 | H | 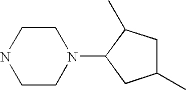 | C₃₆H₄₁N₅O₃ | 591.76 | 592 | Methyl 2-[(3-{[1-(2,4-dimethylcyclopentyl)piperazin-4-yl]methyl}-2-phenylquinolin-4-yl)carbonyl]-1-phenylhydrazinecarboxylate |

The following compounds were prepared from methyl 1-phenyl-2-{[2-phenyl-3-(2-oxo-piperazin-1-ylmethyl)quinolin-4-yl]carbonyl}hydrazinecarboxylate (Example 95) and appropriate ketones according to the method of Description 61.

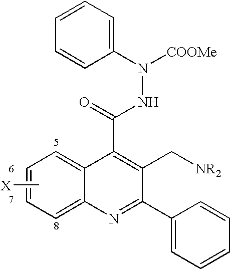

| | | | | | m/z | |
|---|---|---|---|---|---|---|
| Ex. | X | NR₂ | Formula | MWT | (ES⁺) | Name |
| 201 | H | 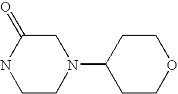 | C₃₄H₃₅N₅O₅ | 593.69 | 594 | methyl 2-{[3-(N-(pyran-4-yl)-2-oxopiperazinylmethyl)-2-phenylquinolin-4-yl]carbonyl}-1-phenylhydrazinecarboxylate |
| 202 | H | 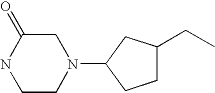 | C₃₆H₃₉N₅O₄ | 605.74 | 606 | methyl 2-{[3-(-(N-(3′-ethylcyclopentyl)-2-oxopiperazinylmethyl)-)-2-phenylquinolin-4-yl]carbonyl}-1-phenylhydrazinecarboxylate |

### EXAMPLE 203

### Methyl 2-{[2-Phenyl-3-(1H-1,2,4-triazol-1-ylmethyl)quinolin-4-yl]carbonyl}-1-phenylhydrazinecarboxylate

To a solution of 1,2,4-triazole (0.031 g, 0.45 mmol) in THF (3 mL) was added NaH (60% dispersion in mineral oil, 0.027 g, 0.9 mmol). After stirring the mixture for 5 min., methyl 2-{[3-(bromomethyl)-2-phenylquinolin-4-yl]carbonyl}-1-phenylhydrazinecarboxylate (Description 16, 0.1 g, 0.24 mmol) was added. The mixture was stirred at RT for 1 h., then the solvent was evaporated under reduced pressure. The residue was partitioned between water (5 mL) and CH₂Cl₂ (2×10 mL) and the organic layer was dried (Na₂SO₄), filtered and the solvent was evaporated under reduced pressure. The residue was purified by silica gel preparative plate chromatography eluting with 40% EtOAc in isohexane to give the title compound as a colorless solid (11 mg). ¹H NMR (500 MHz, CDCl₃): δ 11.11 (1H, br s), 8.14 (1H, d, J 8.2), 8.00 (1H, d, J 8.4), 7.80 (2H, m), 7.64-7.18 (11H, m), 6.96 (1H, s), 6.00 (1H, br s), 5.60 (1H, br s), 3.79 (3H, s). m/z (ES⁺) 479 [M+H⁺].

### EXAMPLE 204

### Methyl 2-{[3-(1H-Benzimidazol-1-ylmethyl)-2-phenylquinolin-4-yl]carbonyl}-1-phenylhydrazinecarboxylate

Prepared from benzimidazole and methyl 2-{[3-(bromomethyl)-2-phenylquinolin-4-yl]carbonyl}-1-phenylhydrazinecarboxylate (Description 16) according to the method of Example 203. ¹H NMR (500 MHz, CDCl₃): 8.5 (1H, br s), 8.24 (1H, d, J 8.4), 7.92 (1H, m), 7.82 (1H, m), 7.31-6.54 (16H, m), 5.49 (1H, br s), 5.28 (1H, br s), 3.63 (3H, br s). m/z (ES⁺) 528 [M+H⁺].

### EXAMPLE 205

### Methyl 2-([2-Phenyl-3-(1H-tetrazol-1-ylmethyl)quinolin-4-yl]carbonyl)-1-phenylhydrazinecarboxylate and Methyl 2-([2-Phenyl-3-(2H-tetrazol-2-ylmethyl)quinolin-4-yl]carbonyl)-1-phenylhydrazinecarboxylate

Prepared from tetrazole and methyl 2-{[3-(bromomethyl)-2-phenylquinolin-4-yl]carbonyl}-1-phenylhydrazinecarboxylate (Description 16) according to the method of Example 203, separating with preparative plate chromatography, eluting with 50% EtOAc/isohexane as eluent. The less polar isomer was methyl 2-([2-phenyl-3-(1H-tetrazol-1-ylmethyl)quinolin-4-yl]carbonyl)-1-phenylhydrazinecarboxylate, ¹H NMR (500 MHz, CDCl₃): δ 9.54 (1H, br s), 8.15 (1H, d, J 8.3), 7.95 (1H, d, J 8.2), 7.83 (1H, m), 7.66 (1H, m), 7.57-7.18 (11H, m), 6.30 (1H, br s), 5.79 (1H, br s), 3.77 (3H, s). m/z (ES⁺) 480 [M+H⁺]. The more polar isomer was methyl 2-([2-phenyl-3-(2H-tetrazol-2-ylmethyl)quinolin-4-yl]carbonyl)-1-phenylhydrazinecarboxylate, ¹H NMR (500 MHz, CDCl₃): δ 9.36 (1H, br s), 8.31 (1H, s) 8.11 (1H, d, J 8.4), 8.00 (1H, m), 7.77 (1H, m), 7.59 (1H, m), 7.42-7.18 (8H, m), 6.95 (2H, d, J 7.8), 6.00 (2H, br s), 3.65 (3H, br s). m/z (ES⁺) 480 [M+H⁺].

### EXAMPLE 206

### Methyl 2-[(3-{[3-(Methoxycarbonyl)-1H-1,2,4-triazol-1-yl]methyl}-2-phenylquinolin-4-yl)carbonyl]-1-phenylhydrazinecarboxylate

Prepared from 3-methoxycarbonyl-1,2,4-triazole and methyl 2-{[3-(bromomethyl)-2-phenylquinolin-4-yl]carbonyl}-1-phenylhydrazinecarboxylate (Description 16) according to the method of Example 203. ¹H NMR (500 MHz, CDCl₃): 10.91 (1H, br s), 8.14 (1H, d, J 8.3), 7.98 (1H, d, J 8.4), 7.81 (1H, t, J 7.5), 7.61 (3H, dd, J 8.0, 8.6), 7.55-7.49 (3H, m), 7.47-7.41 (2H, m), 7.35 (1H, d, J 7.1), 7.20 (2H, d, J 6.3), 7.00 (1H, s), 5.63 (1H, d, J 13.9), 5.30 (1H, s), 3.90 (3H, s), 3.78 (3H, s). m/z (ES⁺) 537 [M+H⁺].

### EXAMPLE 207

### Methyl 1-phenyl-2-({2-phenyl-3-[(4-pyridin-2-yl-1H-1,2,3-triazol-1-yl)methyl]quinolin-4-yl}carbonyl)hydrazinecarboxylate

A solution of methyl 2-{[3-(azidomethyl)-2-phenylquinolin-4-yl]carbonyl}-1-phenylhydrazinecarboxylate in DMSO (Description 56, 0.245M, 0.417 mL, 0.102 mmol) was added to 2-ethynylpyridine (10.3 μL, 0.102 mmol), followed by water (0.104 mL), aqueous copper (II) sulfate (1M, 10.2 μL, 0.0102 mmol) and aqueous sodium ascorbate (1M, 20.4 μL, 0.0204 mmol). The mixture was stirred at RT for 17 h, additional DMSO (0.521 mL) was added and the mixture was stirred at RT for 24 h. The mixture was purified by preparative HPLC (Xterra column, eluting with 15-100% MeCN/NH₄CO₃(aq)) to give the title compound (17.8 mg, 51%). ¹H NMR (400 MHz, DMSO) δ 3.82 (3H, s), 7.30-7.42 (10H, m), 7.53 2H, m), 7.78 (1H, br s), 7.84 (1H, t, 7.7), 7.91-7.96 (2H, m), 8.09 (1H, br s), 8.13 (1H, d, J 8.3), 8.51 (1H, d, J 4.5), 11.91 (1H, s).

The following compounds were prepared according to the method of Example 207, substituting a suitable alkyne for 2-ethynylpyridine.

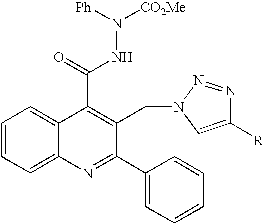

| | | | | m/z | |
|---|---|---|---|---|---|
| | NR₂ | Formula | MWT | (ES⁺) | Name |
| 208 | CO₂Me | C₂₉H₂₄N₆O₅ | 536.55 | 537 | methyl 1-[(4-{[2-(methoxycarbonyl)-2-phenylhydrazino]carbonyl}-2-phenylquinolin-3-yl)methyl]-1H-1,2,3-triazole-4-carboxylate |
| 209 | 3-Pyridyl | C₃₂H₂₅N₇O₃ | 555.6 | 556 | methyl 1-phenyl-2-({2-phenyl-3-[(4-pyridin-3-yl-1H-1,2,3-triazol-1-yl)methyl]quinolin-4-yl}carbonyl)hydrazinecarboxylate |
| 210 | Ac | C₂₉H₂₄N₆O₄ | 520.55 | 521 | methyl 2-({3-[(4-acetyl-1H-1,2,3-triazol-1-yl)methyl]-2-phenylquinolin-4-yl}carbonyl)-1-phenylhydrazinecarboxylate |
| 211 | 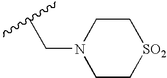 | C₃₂H₃₁N₇O₅S | 625.71 | 626 | methyl 2-{[3-({4-[(1,1-dioxidothiomorpholin-4-yl)methyl]-1H-1,2,3-triazol-1-yl}methyl)-2-phenylquinolin-4-yl]carbonyl}-1-phenylhydrazinecarboxylate |

### EXAMPLE 212

### Methyl 2-{[3-(4′-Hydroxypiperidinylethyl)-2-phenylquinolin-4-yl]carbonyl}-1-phenylhydrazinecarboxylate

Prepared from 2-(1,1-dimethylethyl) 1-methyl 2-[{3-[2-(4-hydroxy)piperidinoethyl]-2-phenylquinolin-4-yl}carbonyl]-1-phenylhydrazinedicarboxylate (Description 41) according to the method of Description 16. m/z (ES⁺) 525 [M+H⁺].

### EXAMPLE 213

### Methyl 2-{[3-(2-Hydroxyethoxy)-2-phenylquinolin-4-yl]carbonyl}-1-phenylhydrazinecarboxylate

Lithium borohydride (2M in THF, 0.4 mL) was added to a solution of methyl 2-{[3-(2-methoxy-2-oxoethoxy)-2-phenylquinolin-4-yl]carbonyl}-1-phenylhydrazinecarboxylate (Example 99, 0.38 g, 0.8 mmol) in THF (8 mL) and the mixture was stirred at RT for 1 h. Saturated aqueous ammonium chloride (4 mL) was added and the mixture was stirred at RT for 5 min. Et₂O (20 mL) was added and the layers were separated. The organic layer was dried (Na₂SO₄), filtered and the solvent was evaporated under reduced pressure. The residue was purified by silica gel column chromatography eluting with 50% EtOAc in isohexane to give the title compound as a colorless solid (240 mg). ¹H NMR (500 MHz, CDCl₃): 8.89 (1H, s), 8.11 (1H, d, J 8.3), 7.98 (1H, d, J 8.3), 7.93 (2H, t, J 3.8), 7.65 (1H, t, J 7.6), 7.58-7.43 (8H, m), 7.39-7.33 (1H, m), 3.87 (3H, s), 3.73 (2H, m), 3.58 (2H, t, J 12.0), 1.60 (1H, br s). m/z (ES⁺) 458 [M+H⁺].

### EXAMPLE 214

### Methyl 2-{[3-(2-Bromoethoxy)-2-phenylquinolin-4-yl]carbonyl}-1-phenylhydrazinecarboxylate

Carbon tetrabromide (0.080 g) and triphenylphosphine (0.064 g) were added to a solution of methyl 2-{[3-(2-hydroxyethoxy)-2-phenylquinolin-4-yl]carbonyl}-1-phenylhydrazinecarboxylate (Example 213, 0.1 g, 0.22 mmol) in CH₂Cl₂ (5 mL) and the mixture was stirred at RT for 14 h. The solvent was evaporated under reduced pressure and the residue was purified by column chromatography eluting with 0-60% EtOAc in isohexane to give the title compound as a colorless solid (32 mg). ¹H NMR (500 MHz, CDCl₃): 8.37 (1H, s), 8.16 (2H, d, J 8.2), 8.00 (2H, d, J 6.5), 7.72 (1H, t, J 7.7), 7.62 (3H, t, J 9.7), 7.52-7.44 (5H, m), 7.34 (1H, t, J 7.4), 3.94-3.92 (5H, m), 3.19 (2H, t, J 6.3). m/z (ES⁺) 522 and 520 [M+H⁺].

### EXAMPLE 215

### 3-[1-(1,1-Dimethylethyl)piperidin-4-yloxy]-N′,2-diphenylquinoline-4-carbohydrazide

Prepared from 3-[1-(1,1-dimethylethyl)piperidin-4-yloxy]-2-phenylquinoline-4-carboxylic acid (Description 44) according to the method of Description 18. m/z (ES⁺) 495 [M+H⁺]

### EXAMPLE 216

### Methyl 2-({3-[1-(1,1-dimethylethyl)piperidin-4-yloxy]-2-phenylquinolin-4-yl}carbonyl)-1-phenylhydrazinecarboxylate

Prepared from 3-[1-(1,1-Dimethylethyl)piperidin-4-yloxy]-N′,2-diphenylquinoline-4-carbohydrazide (Example 215) according to the method of Description 3. m/z (ES⁺) 553 [M+H⁺].

### EXAMPLE 217

### 3-[1-(1,1-Dimethylethyloxycarbonyl)piperidin-4-yloxy]-N′,2-diphenylquinoline-4-carbohydrazide

Prepared from methyl 3-[1-(1,1-dimethylethyloxycarbonyl)piperidin-4-yloxy]-2-phenylquinoline-4-carboxylic acid (Description 46) according to the method of Description 18. m/z (ES⁺) 539 [M+H⁺]

### EXAMPLE 218

### Methyl 2-({3-[1-(1,1-Dimethylethyloxycarbonyl)piperidin-4-yloxy]-2-phenylquinolin-4-yl}carbonyl)-1-phenylhydrazinecarboxylate

Prepared from 3-[1-(1,1-dimethylethyloxycarbonyl)piperidin-4-yloxy]-N′,2-diphenylquinoline-4-carbohydrazide (Example 217) according to the method of Description 3. m/z (ES⁺) 597 [M+H⁺].

### EXAMPLE 219

### Methyl 2-({2-Phenyl-3-(piperidin-4-yloxy)quinolin-4-yl}carbonyl)-1-phenylhydrazinecarboxylate

Prepared from methyl 2-({3-[1-(1,1-dimethylethyloxycarbonyl)piperidin-4-yloxy]-2-phenylquinolin-4-yl}carbonyl)-1-phenylhydrazinecarboxylate (Example 218) according to the method of Description 16. m/z (ES⁺) 497 [M+H⁺]

### EXAMPLE 220

### Methyl 2-[(3-(4-oxo-1-(4-pyranyl)-piperidinyl)-2-phenylquinolin-4-yl)carbonyl]-1-phenylhydrazinecarboxylate

Prepared from methyl 2-({2-phenyl-3-(piperidin-4-yloxy)quinolin-4-yl}carbonyl)-1-phenylhydrazinecarboxylate (Example 219) according to the method of Description 61. m/z (ES⁺) 581 [M+H⁺]

### EXAMPLE 221

### Methyl 1-phenyl-2-{[2-phenyl-3-({[1-(tetrahydro-2H-pyran-4-yl)azetidin-3-yl]oxy}methyl)quinolin-4-yl]carbonyl}hydrazinecarboxylate

Prepared from 2-(1,1-dimethylethyl) 1-methyl 2-{[3-(bromomethyl)-2-phenylquinolin-4-yl]carbonyl}-1-phenylhydrazinedicarboxylate (Description 15) and 1,1-dimethylethyl 3-hydroxy-1-azetidinecarboxylate according to the methods of Example 203, followed by treatment with TFA according to the method of Description 16 and reductive amination with tetrahydro-4H-pyran-4-one according to the method of Description 61. m/z (ES⁺) 567 [M+H⁺].

### EXAMPLE 222

### Methyl 1-phenyl-2-({2-Phenyl-3-[(4-piperidinyloxy)methyl]quinolin-4-yl}carbonyl)hydrazinecarboxylate

Prepared from 2-(1,1-dimethylethyl) 1-methyl 2-{[3-(bromomethyl)-2-phenylquinolin-4-yl]carbonyl}-1-phenylhydrazinedicarboxylate (Description 15) and 1,1-dimethylethyl 4-hydroxy-1-piperidinecarboxylate according to the method of Example 203 followed by treatment with TFA according to the method of Description 16. m/z (ES⁺) 511 [M+H⁺].

### EXAMPLE 223

### Methyl 1-phenyl-2-{[2-phenyl-3-({[1-(tetrahydro-2H-pyran-4-yl)-4-piperidinyl]oxy}methyl)quinolin-4-yl]carbonyl}hydrazinecarboxylate

Prepared from methyl 1-phenyl-2-({2-phenyl-3-[(4-piperidinyloxy)methyl]quinolin-4-yl}carbonyl)hydrazinecarboxylate (Example 222) and tetrahydro-4H-pyran-4-one according to the method of Description 61. m/z (ES⁺) 595 [M+H⁺].

### EXAMPLE 224

### Methyl 2-({3-[(1-Ethyl-1H-1,2,4-triazol-5-yl)methoxymethyl]-2-phenylquinolin-4-yl}carbonyl)-1-phenylhydrazinecarboxylate

Prepared from (2-ethyl-2H-[1,2,4]triazol-3-yl)-methanol and methyl 2-{[3-(bromomethyl)-2-phenylquinolin-4-yl]carbonyl}-1-phenylhydrazinecarboxylate (Description 16) according to the method of Example 203. ¹H NMR (500 MHz, CDCl₃): δ 10.06 (1H, s), 8.12 (1H, d, J 8.4), 7.99 (1H, d, J 8.3), 7.75 (1H, t, J 7.7), 7.65 (1H, s), 7.61-7.55 (5H, m), 7.47-7.43 (5H, m), 7.34 (1H, t, J 7.3), 4.90 (2H, br s), 4.58 (2H, br s), 4.05 (2H, q, J 7.1), 3.85 (3H, s), 1.38 (3H, t, J 7.3). m/z (ES⁺) 537 [M+H⁺].

### EXAMPLE 225

### Methyl 2-{[3-(Methylthiomethyl)-2-phenylquinolin-4-yl]carbonyl}-1-phenylhydrazinecarboxylate

Methyl 2-{[3-(bromomethyl)-2-phenylquinolin-4-yl]carbonyl}-1-phenylhydrazinecarboxylate (Description 16, 417 mg, 0.71 mmol) in DMSO (10 mL) was heated to 110° C. for 3 h. The mixture was cooled, water and EtOAc were added and the layers were separated. The organic layer was washed with brine, dried (MgSO₄) and the solvent was evaporated under reduced pressure. The residue was purified by column chromatography on silica gel, eluting with EtOAc/hexane (10:90) to give the title compound (62 mg). m/z (ES⁺) 458 [M+H⁺].

### EXAMPLE 226

### Methyl 2-{[3-(Methylthiosulfonylmethyl)-2-phenylquinolin-4-yl]carbonyl}-1-phenylhydrazinecarboxylate

3-Chlorobenzenecarboperoxoic acid (77%, 22 mg) was added to a solution of methyl 2-{[3-(methylthiomethyl)-2-phenylquinolin-4-yl]carbonyl}-1-phenylhydrazinecarboxylate (Example 225, mg, 0.055 mmol) in CH₂Cl₂ (1 mL) and the mixture was stirred at RT overnight. The mixture was washed with aqueous sodium metabisulfite, aqueous NaHCO₃ (saturated) and brine, dried (MgSO₄) and the solvent was evaporated under reduced pressure. The residue was purified by preparative HPLC to give the title compound (14 mg). m/z (ES⁺) 490 [M+H⁺].

### EXAMPLE 227

### Methyl 2-{[3-(Methylsulfoxidylmethyl)-2-phenylquinolin-4-yl]carbonyl}-1-phenylhydrazinecarboxylate

Oxone™ (38 mg) was added to methyl 2-{[3-(methylthiomethyl)-2-phenylquinolin-4-yl]carbonyl}-1-phenylhydrazinecarboxylate (Example 225, 28 mg, 0.06 mmol) and wet alumina (0.6 g) in CH₂Cl₂ (5 mL) and the mixture was heated under reflux overnight. The mixture was cooled, filtered and the solvent was evaporated under reduced pressure. The residue was purified by preparative HPLC to give the title compound (2 mg). m/z (ES⁺) 474 [M+H⁺].

### EXAMPLE 228

### Methyl 2-[(3-{[1-(Pyran-4-yl)piperidin-4-yl]methyl}-2-phenylquinolin-4-yl)carbonyl]-1-phenylhydrazinecarboxylate

Prepared from methyl 2-({3-[(piperidin-4-yl)methyl]-2-phenylquinolin-4-yl}carbonyl)-1-phenylhydrazinecarboxylate (Description 55) and tetrahydro-4H-pyran-4-one according to the method of Description 61. m/z (ES⁺) 579 [M+H⁺].

The following compounds were prepared from 3-(5,6-dihydro[1,2,4]triazolo[1,5-a]pyrazin-7(8H)-ylmethyl)-2-phenylquinoline-4-carboxylic acid (Description 35) and a suitable hydrazine according to the method of Description 18.

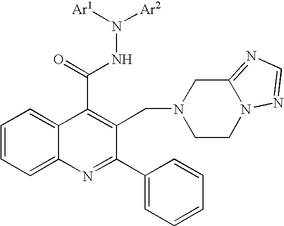

| | | | | | m/z | |
|---|---|---|---|---|---|---|
| Ex. | Ar¹ | Ar² | Formula | MWT | (ES⁺) | Name |
| 229 | Ph | Ph | C₃₄H₂₉N₇O | 551.66 | 552 | 3-(5,6-dihydro[1,2,4]triazolo[1,5-a]pyrazin-7(8H)-ylmethyl)-N′,N′,2-triphenylquinoline-4-carbohydrazide |
| 230 | Ph | 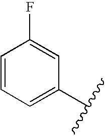 | C₃₄H₂₈FN₇O | 569.65 | 570 | 3-(5,6-dihydro[1,2,4]triazolo[1,5-a]pyrazin-7(8H)-ylmethyl)-N′-(3-fluorophenyl)-N′,2-diphenylquinoline-4-carbohydrazide |
| 231 | 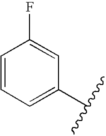 | 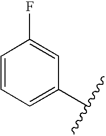 | C₃₄H₂₇F₂N₇O | 587.64 | 588 | 3-(5,6-dihydro[1,2,4]triazolo[1,5-a]pyrazin-7(8H)-ylmethyl)-N′,N′-bis(3-fluorophenyl)-2-phenylquinoline-4-carbohydrazide |
| 232 | Ph | Et | C₃₀H₂₉N₇O | 503.61 | 504 | 3-(5,6-dihydro[1,2,4]triazolo[1,5-a]pyrazin-7(8H)-ylmethyl)-N′-ethyl-N′,2-diphenylquinoline-4-carbohydrazide |
| 233 | Ph | 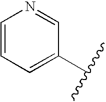 | C₃₃H₂₈N₈O | 552.64 | 553 | 3-(5,6-dihydro[1,2,4]triazolo[1,5-a]pyrazin-7(8H)-ylmethyl)-N′,2-diphenyl-N′-pyridin-3-ylquinoline-4-carbohydrazide |
| 234 | Ph | 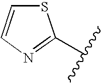 | C₃₁H₂₆N₈OS | 558.67 | 559 | 3-(5,6-dihydro[1,2,4]triazolo[1,5-a]pyrazin-7(8H)-ylmethyl)-N′,2-diphenyl-N′-1,3-thiazol-2-ylquinoline-4-carbohydrazide |

### EXAMPLE 235

### 1,1-Dimethylethyl 4-[(2-Phenyl-4-({2,2-diphenylhydrazino}carbonyl)quinolin-3-yl)methyl]piperidine-1-carboxylate

Prepared from 3-({1-[(1,1-dimethylethoxy)carbonyl]piperidin-4-yl}methyl-2-phenylquinoline-4-carboxylic Acid (Description 49) and N,N-diphenylhydrazine according to the method of Description 51. m/z (ES⁺) 613 [M+H⁺].

1-17. (canceled)

18. A compound of the Formula (I): 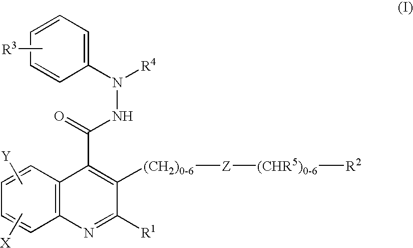
wherein:
R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulfur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;
or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a}, where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen;
or, when R¹ is CONR^{a}R^{b} or SO₂NR^{a}R^{b}, R^{a}, R^{b} and the nitrogen atom to which they are attached form a piperidine, piperazine, pyrrolidine, morpholine, aziridine, azetidine or azepine ring, optionally substituted by 1, 2 or 3 groups independently chosen from hydroxy, C₁₋₄alkyl or C₁₋₄alkoxy;
R² is hydrogen, hydroxy, halogen, CN or CO₂H;
or R² is C₁₋₆alkyl, C₂₋₆alkenyl, C₂₋₆alkynyl, C₃₋₈cycloalkyl, C₁₋₆alkoxy, C(O)OC₁₋₆alkyl, Het, heteroaryl or aryl, optionally substituted by C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, OHet, C(O)C₁₋₆alkyl, C(O)OC₁₋₆alkyl, S(O)₂C₁₋₆alkyl, hydroxy or one to eight halogen atoms, where OHet is optionally substituted by C₁₋₄alkyl;
or R² is NR^{d}R^{e},
where R^{d} and R^{e} are independently selected from hydrogen, C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl and S(O)₂C₁₋₆alkyl, optionally substituted by NR^{f}R^{g}, halogen, C₁₋₆alkyl, hydroxy, C₁₋₆alkoxy, CN or CO₂H,
where R^{f} and R^{g} are independently selected from hydrogen and C₁₋₆alkyl,
or R^{d} and R^{e}, together with the nitrogen atom to which they are attached, form a nitrogen-containing 3- to 7-membered heterocycle optionally containing a further nitrogen, oxygen or sulfur atom or a S(O) or S(O)₂ group;
which heterocycle is optionally substituted by 1, 2 or 3 groups independently chosen from C₁₋₆alkyl, oxo, halogen and (CH₂)₀₋₆R^{h}, where R^{h} is hydroxy, C₁₋₆alkoxy, C₃₋₈cycloalkyl, bicyclo[3.3.1]non-9-yl, C(O)Rⁱ, C(O)ORⁱ, NRⁱR^{j}, CONRⁱR^{j}, Het, heteroaryl, aryl, SO₂Het, SO₂heteroaryl, SO₂(CH₂)₀₋₃aryl or SO₂C₁₋₆alkyl, where C₁₋₆alkyl and C₃₋₈cycloalkyl, either as separate moieties or as part of a moiety, and heteroaryl, Het and aryl are optionally substituted by 1 to 8 halogen atoms, hydroxy, methoxy, oxo, C₁₋₄alkyl, CF₃ or CH₂CF₃, and C₃₋₈cycloalkyl is optionally fused or spiro-fused to Het, heteroaryl or aryl, where Het and aryl, either as separate moieties or as part of a moiety, are optionally substituted by C₁₋₆alkyl, hydroxy or halogen, and optionally fused to Het, heteroaryl, aryl or C₃₋₈cycloalkyl, and where Rⁱ and R^{j} are independently selected from hydrogen, C₁₋₆alkyl, (CH₂)₀₋₃aryl, (CH₂)₀₋₃heteroaryl, (CH₂)₀₋₃ Het and C(O)C₁₋₆ alkyl;
which heterocycle is further optionally fused or spiro-fused to Het, heteroaryl, aryl or C₃₋₈cycloalkyl, optionally substituted by C₁₋₆alkyl, C₃₋₈cycloalkyl, hydroxy, C₁₋₆alkoxy, halogen, oxo, C(O)OC₁₋₆alkyl, Het, CF₃, CHF₂ or CH₂CF₃;
which heterocycle is further optionally bridged by —(CH₂)₁₋₂—;
R³ is hydrogen, hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, optionally substituted by 1 to 8 halogen atoms;
R⁴ is hydrogen, C₁₋₆alkyl, C(O)C₁₋₆alkyl, C(O)OC₁₋₆alkyl, (CH₂)₀₋₃-phenyl or heteroaryl, optionally substituted by 1 to 3 halogen atoms;
R⁵ is hydrogen, C₁₋₆alkyl, hydroxy or C₁₋₆alkoxy;
X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NR^{k}R^{m}, CO₂R^{k} and (CH₂)₀₋₄NRⁿR^{p}, SO₂R^{k}R^{m}, SO₂NR^{k}R^{m}, optionally substituted by 1 to 8 halogen atoms;
R^{k} and R^{m} are independently chosen from hydrogen, C₁₋₆alkyl, C₂₋₆alkenyl, C₂₋₆alkynyl, C₃₋₈cycloalkyl and (CH₂)₀₋₄aryl;
Rⁿ and R^{p} are independently chosen from hydrogen, C₁₋₆alkyl, C₂₋₆alkenyl, C₂₋₆alkynyl, C₃₋₈cycloalkyl, aryl, C(O)R^{q}, COOR^{q} and S(O)₂R^{q};
R^{q} is hydrogen, C₁₋₆alkyl, C₂₋₆alkenyl, C₂₋₆alkynyl, C₃₋₈cycloalkyl or (CH₂)₀₋₄aryl;
Z is a bond, O, S, SO, SO₂, CO, NR^{c}SO₂ or SO₂NR^{c}, NR^{c}, NR^{c}CO or CONR^{c},
where R^{c} is hydrogen or C₁₋₆alkyl, optionally substituted by one to four halogen atoms;
or a pharmaceutically acceptable salt thereof.

19. The compound of claim 18 of the Formula (Io): 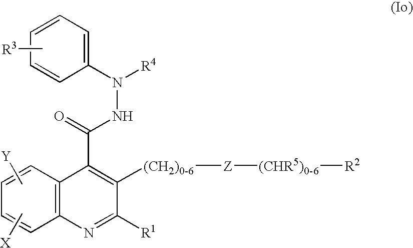
wherein:
R¹ is an aryl or heteroaryl ring, wherein aryl is phenyl or naphthyl and heteroaryl is a 5-membered unsaturated ring containing 1, 2, 3 or 4 nitrogen atoms and/or, an oxygen or sulfur atom provided no more than two nitrogen atoms are present, or a 6-membered unsaturated ring containing 1, 2 or 3 nitrogen atoms, said ring being optionally substituted by one, two or three groups independently chosen from hydroxy, halogen, nitro, cyano, amino, CF₃, C₁₋₄alkyl, C₂₋₄alkenyl and C₂₋₄alkynyl;
or R¹ is OR^{a}, C(O)R^{a}, COOR^{a}, S(O)₂R^{a}, NR^{a}R^{b}, CONR^{a}R^{b}, SO₂NR^{a}R^{b} or a non-aromatic ring of 3 to 8 ring atoms where said ring optionally contains a double bond, and where said ring optionally contains 1, 2 or 3 heteroatoms selected from N, O or S or a group C(O), S(O), S(O)₂, NH or NC₁₋₄alkyl, and where said ring is also optionally fused to aryl, and where said ring is further optionally bridged by (CH₂)₁₋₄, and where said ring is also optionally substituted by 1, 2 or 3 groups independently chosen from hydroxy, halogen, NO₂, CN, NH₂, CF₃, C₁₋₄alkyl, C₂₋₄alkenyl, C₂₋₄alkynyl, OR^{a} and CO₂R^{a}, where R^{a} and R^{b} are independently selected from hydrogen, C₁₋₆alkyl, C₂₋₆alkenyl, C₂₋₆alkynyl, C₃₋₈cycloalkyl and (CH₂)₀₋₃aryl, optionally substituted by hydroxy or halogen;
or, when R¹ is CONR^{a}R^{b} or SO₂NR^{a}R^{b}, R^{a}, R^{b} and the nitrogen atom to which they are attached form a piperidine, piperazine, pyrrolidine, morpholine, aziridine, azetidine or azepine ring, optionally substituted by 1, 2 or 3 groups independently chosen from hydroxy, C₁₋₄alkyl or C₁₋₄alkoxy;
R² is hydrogen, hydroxy, halogen, CN or CO₂H;
or R² is C₁₋₆alkyl, C₂₋₆alkenyl, C₂₋₆alkynyl, C₃₋₈cycloalkyl, C₁₋₆alkoxy, C(O)OC₁₋₆alkyl, Het, heteroaryl or aryl, optionally substituted by C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl, S(O)₂C₁₋₆alkyl or one to eight halogen atoms;
or R² is NR^{d}R^{e},
where R^{d} and R^{e} are independently selected from hydrogen, C₁₋₆alkyl, C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl and S(O)₂C₁₋₆alkyl, optionally substituted by NR^{f}R^{g}, halogen, C₁₋₆alkyl, hydroxy, C₁₋₆alkoxy, CN or CO₂H,
where R^{f} and R^{g} are independently selected from hydrogen and C₁₋₆alkyl,
or R^{d} and R^{e}, together with the nitrogen atom to which they are attached, form a saturated nitrogen-containing 3- to 7-membered heterocycle optionally containing a further nitrogen, oxygen or sulfur atom or a S(O) or S(O)₂ group;
which heterocycle is optionally substituted by 1, 2 or 3 groups independently chosen from C₁₋₆alkyl, oxo, halogen and (CH₂)₀₋₆R^{h}, where R^{h} is hydroxy, C₁₋₆alkoxy, C₃₋₈cycloalkyl, C(O)Rⁱ, C(O)ORⁱ, NRⁱR^{j}, CONRⁱR^{j}, Het, heteroaryl, aryl, SO₂Het, SO₂heteroaryl, SO₂(CH₂)₀₋₃aryl or SO₂C₁₋₆alkyl, where C₁₋₆alkyl and C₃₋₈cycloalkyl, either as separate moieties or as part of a moiety, are optionally substituted by 1 to 8 halogen atoms or C₁₋₄alkyl, and C₃₋₈cycloalkyl is optionally fused or spiro-fused to Het, heteroaryl or aryl, where Het and aryl, either as separate moieties or as part of a moiety, are optionally substituted by C₁₋₆alkyl, hydroxy or halogen, and optionally fused to Het, heteroaryl, aryl or C₃₋₈cycloalkyl, and where Rⁱ and R^{j} are independently selected from hydrogen, C₁₋₆alkyl, (CH₂)₀₋₃aryl, (CH₂)₀₋₃heteroaryl, (CH₂)₀₋₃ Het and C(O)C₁₋₆ alkyl;
which heterocycle is further optionally fused or spiro-fused to Het, heteroaryl, aryl or C₃₋₈cycloalkyl, optionally substituted by C₁₋₆alkyl, C₃₋₈cycloalkyl, hydroxy, C₁₋₆alkoxy, halogen, oxo, CF₃, CHF₂ or CH₂CF₃;
which heterocycle is further optionally bridged by —(CH₂)₁₋₂—;
R³ is hydrogen, hydroxy, halogen, NO₂, CN, NH₂, C₁₋₄alkyl, C₂₋₄alkenyl, C₂₋₄alkynyl, C₁₋₄alkoxy or C(O)OC₁₋₆alkyl, optionally substituted by 1 to 8 halogen atoms;
R⁴ is hydrogen, C₁₋₆alkyl, C(O)C₁₋₆alkyl, C(O)OC₁₋₆alkyl or (CH₂)₀₋₃phenyl;
R⁵ is hydrogen, C₁₋₆alkyl, hydroxy or C₁₋₆alkoxy;
X and Y are independently chosen from hydrogen, hydroxy, nitro, cyano, CF₃, halogen, C₁₋₆alkyl, C₂₋₆alkenyl, C₂₋₆alkynyl, C₃₋₈cycloalkyl, C₁₋₆alkoxy, C(O)NR^{k}R^{m}, CO₂R^{k} and (CH₂)₀₋₄NRⁿR^{p}, SO₂R^{k}, SO₂NR^{k}R^{m}, optionally substituted by 1 to 8 halogen atoms;
R^{k} and R^{m} are independently chosen from hydrogen, C₁₋₆alkyl, C₂₋₆alkenyl, C₂₋₆alkynyl, C₃₋₈cycloalkyl and (CH₂)₀₋₄aryl;
Rⁿ and R^{p} are independently chosen from hydrogen, C₁₋₆alkyl, C₂₋₆alkenyl, C₂₋₆alkynyl, C₃₋₈cycloalkyl, aryl, C(O)R^{q}, COOR^{q} and S(O)₂R^{q};
R^{q} is hydrogen, C₁₋₆alkyl, C₂₋₆alkenyl, C₂₋₆alkynyl, C₃₋₈cycloalkyl or (CH₂)₀₋₄aryl;
Z is a bond, O, S, SO, SO₂, CO, NR^{c}SO₂ or SO₂NR^{c}, NR^{c}, NR^{c}CO or CONR^{c}, where R^{c} is hydrogen or C₁₋₆alkyl, optionally substituted by one to four halogen atoms;
or a pharmaceutically acceptable salt thereof.

20. The compound of claim 18 wherein R¹ is aryl or heteroaryl, optionally substituted by 1, 2 or 3 groups independently chosen from hydroxy, halogen, nitro, cyano, amino, CF₃, C₁₋₄alkyl, C₂₋₄alkenyl or C₂₋₄alkynyl.

21. The compound of claim 18 wherein R² is CN, CO₂H, C(O)OC₁₋₆alkyl, Het, heteroaryl, aryl or NR^{d}R^{e}, where Het, heteroaryl and aryl are optionally substituted by C₁₋₆alkyl C₂₋₆alkenyl, C₂₋₆alkynyl, C₃₋₈cycloalkyl, (CH₂)₀₋₃aryl, (CH₂)₀₋₃heteroaryl, (CH₂)₀₋₃Het, C(O)C₁₋₆alkyl, C(O)OC₁₋₆alkyl, S(O)₂C₁₋₆alkyl or one to eight halogen atoms, and where NR^{d}R^{e} is as defined in claim 1.

22. The compound of claim 18 wherein R³ is hydrogen, hydroxy, halogen, C₁₋₄alkyl, C₂₋₄alkenyl or C₂₋₄alkynyl, optionally substituted by 1 to 8 halogen atoms.

23. The compound of claim 18 wherein R⁴ is C(O)C₁₋₆alkyl, C(O)OC₁₋₆alkyl, phenyl or benzyl.

24. The compound of claim 18 wherein R⁵ is hydrogen, C₁₋₄alkyl or hydroxy.

25. The compound of claim 18 wherein X is hydrogen, hydroxy, nitro, cyano, CF₃, halogen, C₁₋₆alkyl, C₂₋₆alkenyl, C₂₋₆alkynyl, C₃₋₈cycloalkyl or C₁₋₆alkoxy.

26. The compound of claim 18 wherein Y is hydrogen, hydroxy, nitro, cyano, CF₃, halogen, C₁₋₆alkyl, C₂₋₆alkenyl, C₂₋₆alkynyl, C₃₋₈cycloalkyl or C₁₋₆alkoxy.

27. The compound of claim 18 wherein Z is a bond, O or S.

28. The compound of claim 18 which is selected from the group consisting of the compound of formula (Ia): 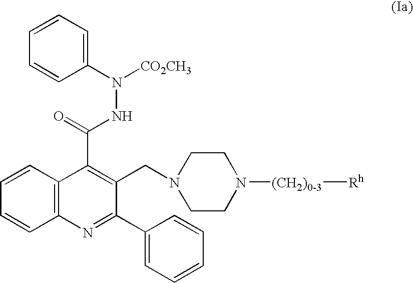
wherein (CH₂)₀₋₃R^{h} is C₁₋₆alkyl;
the compound of formula (Ib): 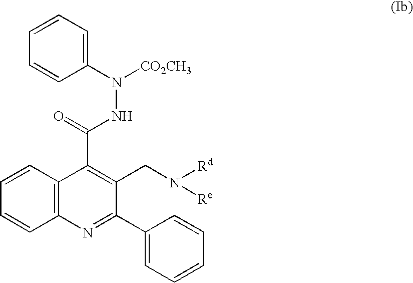
the compound of formula (Ic): 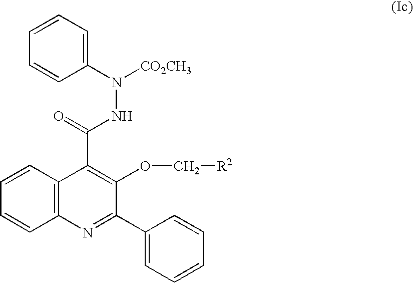
the compound of formula (Id): 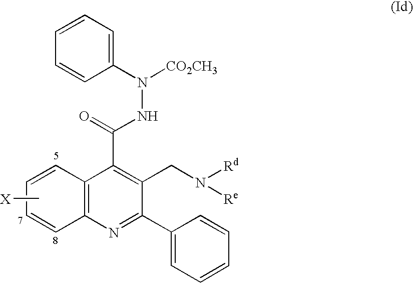
wherein X is selected from NO₂, CN, CF₃ and halogen,
the compound of formula (Ie): 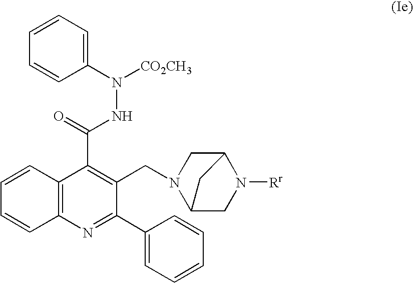
wherein R^{r} is hydrogen, C₁₋₆alkyl or (CH₂)₀₋₆R^{h},
and where C₁₋₆alkyl, C₃₋₈cycloalkyl and heteroaryl are optionally substituted by 1 to 8 halogen atoms or C₁₋₄alkyl;
the compound of formula (If): 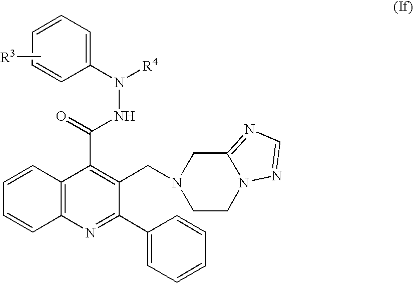
the compound of formula (Ig): 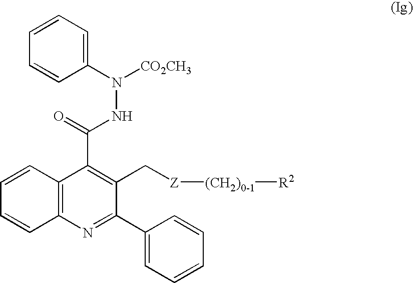
the compound of formula (Ih): 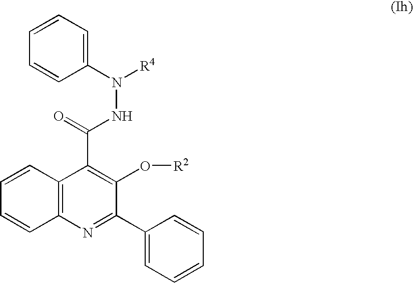
or a pharmaceutically acceptable salt thereof.

29. A compound which is selected from the group consisting of:
tert-Butyl-4-[(4-{[2-(methoxycarbonyl)-2-phenylhydrazino]carbonyl}-2-phenylquinolin-3-yl)methyl]piperazine-1-carboxylate;
Methyl 1-phenyl-2-{[2-phenyl-3-(piperazin-1-ylmethyl)quinolin-4-yl]carbonyl}hydrazinecarboxylate;
Methyl 2-[(3-{[4-(phenylsulfonyl)piperazin-1-yl]methyl}-2-phenylquinolin-4-yl)carbonyl]-1-phenylhydrazine carboxylate;
Methyl 2-({3-[(4-acetylpiperazin-1-yl)methyl]-2-phenylquinolin-4-yl}carbonyl)-1-phenylhydrazinecarboxylate;
Methyl 2-({3-[(4-acetylpiperazin-1-yl)methyl]-2-phenylquinolin-4-yl}carbonyl)-1-phenylhydrazinecarboxylate;
methyl 1-phenyl-2-({2-phenyl-3-[(4-propionylpiperazin-1-yl)methyl]quinolin-4-yl}carbonyl)hydrazinecarboxylate;
methyl 2-({3-[(4-isobutyrylpiperazin-1-yl)methyl]-2-phenylquinolin-4-yl}carbonyl)-1-phenylhydrazinecarboxylate;
methyl 2-[(3-{[4-(2,2-dimethyl propanoyl)piperazin-1-yl]methyl}-2-phenylquinolin-4-yl)carbonyl]-1-phenylhydrazinecarboxylate;
methyl 2-({3-[(4-benzoylpiperazin-1-yl)methyl]-2-phenylquinolin-4-yl}carbonyl)-1-phenylhydrazinecarboxylate;
methyl 1-phenyl-2-[(2-phenyl-3-{[4-(phenylacetyl)piperazin-1-yl]methyl}quinolin-4-yl)carbonyl]hydrazinecarboxylate;
methyl 4-[(4-{[2-(methoxycarbonyl)-2-phenylhydrazino]carbonyl}-2-phenylquinolin-3-yl)methyl]piperazine-1-carboxylate;
isobutyryl 4-[(4-{[2-(methoxy carbonyl)-2-phenylhydrazino]carbonyl}-2-phenylquinolin-3-yl)methyl]piperazine-1-carboxylate;
methyl 1-phenyl-2-[(2-phenyl-3-{[4-(pyridin-4-ylsulfonyl)piperazin-1-yl]methyl}quinolin-4-yl)carbonyl]hydrazinecarboxylate;
benzyl 4-[(4-{[2-(methoxycarbonyl)-2-phenylhydrazino]carbonyl}-2-phenylquinolin-3-yl)methyl]piperazine-1-carboxylate;
methyl 2-[(3-{[4-(methylsulfonyl)piperazin-1-yl]methyl}-2-phenylquinolin-4-yl)carbonyl]-1-phenylhydrazinecarboxylate;
methyl 2-[(3-{[4-(ethylsulfonyl)piperazin-1-yl]methyl}-2-phenylquinolin-4-yl)carbonyl]-1-phenylhydrazinecarboxylate;
methyl 1-phenyl-2-[(2-phenyl-3-{[4-(propylsulfonyl)piperazin-1-yl]methyl}quinolin-4-yl)carbonyl]hydrazinecarboxylate;
methyl 1-phenyl-2-{[2-phenyl-3-({4-[(trifluoromethyl)sulfonyl]piperazin-1-yl}methyl)quinolin-4-yl]carbonyl}hydrazinecarboxylate;
methyl 2-[(3-{[4-(phenylsulfonyl)piperazin-1-yl]methyl}-2-phenylquinolin-4-yl)carbonyl]-1-phenylhydrazine carboxylate;
methyl 2-[(3-{[4-(benzylsulfonyl)piperazin-1-yl]methyl}-2-phenylquinolin-4-yl)carbonyl]-1-phenylhydrazinecarboxylate;
methyl 1-phenyl-2-[(2-phenyl-3-{[4-(2-thienylsulfonyl)piperazin-1-yl]methyl}quinolin-4-yl)carbonyl]hydrazinecarboxylate;
methyl 2-{[3-({4-[(6-chloropyridin-3-yl)sulfonyl]piperazin-1-yl}methyl)-2-phenylquinolin-4-yl]carbonyl}-1-phenylhydrazinecarboxylate;
methyl 1-phenyl-2-[(2-phenyl-3-{[4-(pyridin-3-ylsulfonyl)piperazin-1-yl]methyl}quinolin-4-yl)carbonyl]hydrazinecarboxylate;
methyl 1-phenyl-2-[(2-phenyl-3-{[4-(pyridin-2-ylsulfonyl)piperazin-1-yl]methyl}quinolin-4-yl)carbonyl]hydrazinecarboxylate;
methyl 2-{[3-({4-[(1-methyl-1-pyrazol-4-yl)sulfonyl]piperazin-1-yl}methyl)-2-phenylquinolin-4-yl]carbonyl}-1-phenylhydrazinecarboxylate;
Methyl 2-[(3-{[4-(tetrahydropyran-4-yl)piperazin-1-yl]methyl}-2-phenylquinolin-4-yl)carbonyl]-1-phenylhydrazine carboxylate;
methyl 2-({3-[(4-cyclopentylpiperazin-1-yl)methyl]-2-phenylquinolin-4-yl}carbonyl)-1-phenylhydrazinecarboxylate;
methyl 2-[(3-{[4-(3-methylcyclohexyl)piperazin-1-yl]methyl}-2-phenylquinolin-4-yl)carbonyl]-1-phenylhydrazinecarboxylate;
methyl 2-[(3-{[4-(1,4-dioxaspiro[4.5]dec-8-yl)piperazin-1-yl]methyl}-2-phenylquinolin-4-yl)carbonyl]-1-phenylhydrazinecarboxylate;
Methyl 2-{[3-({4-[2-(dimethylamino)-2-oxoethyl]piperazin-1-yl}methyl)-2-phenylquinolin-4-yl]carbonyl}-1-phenylhydrazinecarboxylate;
methyl 2-[(3-{[4-(cyclopropylmethyl)piperazin-1-yl]methyl}-2-phenylquinolin-4-yl)carbonyl]-1-phenylhydrazinecarboxylate;
methyl 2-({3-[(4-methylpiperazin-1-yl)methyl]-2-phenyl quinolin-4-yl}carbonyl)-1-phenylhydrazinecarboxylate;
methyl 2-({3-[(4-isopropylpiperazin-1-yl)methyl]-2-phenylquinolin-4-yl}carbonyl)-1-phenylhydrazinecarboxylate;
methyl 2-[(3-{[methyl(1-methylpiperidin-4-yl)amino]methyl}-2-phenylquinolin-4-yl)carbonyl]-1-phenylhydrazinecarboxylate;
methyl 1-phenyl-2-{[2-phenyl-3-(thiomorpholin-4-ylmethyl)quinolin-4-yl]carbonyl}hydrazinecarboxylate;
methyl 2-[(3-{[methyl(2-pyridin-2-ylethyl)amino]methyl}-2-phenylquinolin-4-yl)carbonyl]-1-phenylhydrazinecarboxylate;
methyl 1-phenyl-2-{[2-phenyl-3-(1,3-thiazolidin-3-ylmethyl)quinolin-4-yl]carbonyl}hydrazinecarboxylate;
methyl 2-[(3-{[(2R)-2-(methoxymethyl)pyrrolidin-1-yl]methyl}-2-phenylquinolin-4-yl)carbonyl]-1-phenylhydrazinecarboxylate;
methyl 1-phenyl-2-({2-phenyl-3-[(4-pyridin-2-ylpiperazin-1-yl)methyl]quinolin-4-yl}carbonyl)hydrazinecarboxylate;
methyl 2-({3-[(4-hydroxypiperidin-1-yl)methyl]-2-phenyl quinolin-4-yl}carbonyl)-1-phenylhydrazinecarboxylate;
methyl 2-[(3-{[[3-(dimethylamino)propyl](methyl)amino]methyl}-2-phenylquinolin-4-yl)carbonyl]-1-phenylhydrazinecarboxylate;
methyl 1-phenyl-2-({2-phenyl-3-[(4-phenylpiperazin-1-yl)methyl]quinolin-4-yl}carbonyl)hydrazinecarboxylate;
methyl 2-{[3-(1,4′-bipiperidin-1′-ylmethyl)-2-phenyl quinolin-4-yl]carbonyl}-1-phenylhydrazinecarboxylate;
ethyl 1-[(4-{[2-(methoxycarbonyl)-2-phenylhydrazino]carbonyl}-2-phenylquinolin-3-yl)methyl]piperidine-4-carboxylate;
methyl 2-[(3-{[4-(aminocarbonyl)piperidin-1-yl]methyl}-2-phenylquinolin-4-yl)carbonyl]-1-phenylhydrazinecarboxylate;
methyl 2-({3-[(4-benzylpiperazin-1-yl)methyl]-2-phenylquinolin-4-yl}carbonyl)-1-phenylhydrazinecarboxylate;
methyl 2-[(3-{[(2S)-2-(hydroxymethyl)pyrrolidin-1-yl]methyl}-2-phenylquinolin-4-yl)carbonyl]-1-phenylhydrazinecarboxylate;
methyl 2-[(3-{[ethyl(2-hydroxyethyl)amino]methyl}-2-phenylquinolin-4-yl)carbonyl]-1-phenylhydrazinecarboxylate;
methyl 2-[(3-{[4-(2-hydroxyethyl)piperazin-1-yl]methyl}-2-phenylquinolin-4-yl)carbonyl]-1-phenylhydrazinecarboxylate;
methyl 2-({3-[(4-ethylpiperazin-1-yl)methyl]-2-phenyl quinolin-4-yl}carbonyl)-1-phenylhydrazinecarboxylate;
methyl 2-[(3-{[(2-cyanoethyl)(methyl)amino]methyl}-2-phenylquinolin-4-yl)carbonyl]-1-phenylhydrazinecarboxylate;
methyl 2-{[3-(3,4-dihydroisoquinolin-2(1H)-ylmethyl)-2-phenylquinolin-4-yl]carbonyl}-1-phenylhydrazinecarboxylate;
methyl 2-({3-[(4-cyclohexylpiperazin-1-yl)methyl]-2-phenylquinolin-4-yl}carbonyl)-1-phenylhydrazinecarboxylate;
methyl 1-phenyl-2-({2-phenyl-3-[(4-pyrrolidin-1-ylpiperidin-1-yl)methyl]quinolin-4-yl}carbonyl)hydrazinecarboxylate;
methyl 2-({3-[(3-oxopiperazin-1-yl)methyl]-2-phenyl quinolin-4-yl}carbonyl)-1-phenylhydrazinecarboxylate;
methyl 2-({3-[(3-hydroxypyrrolidin-1-yl)methyl]-2-phenylquinolin-4-yl}carbonyl)-1-phenylhydrazinecarboxylate;
methyl 2-({3-[(2-oxo-1,3,8-triazaspiro[4.5]dec-8-yl)methyl]-2-phenylquinolin-4-yl}carbonyl)-1-phenylhydrazinecarboxylate;
1-[(4-{[2-(methoxycarbonyl)-2-phenylhydrazino]carbonyl}-2-phenylquinolin-3-yl)methyl]piperidine-3-carboxylic acid;
methyl 2-[(3-{[ethyl(pyridin-4-ylmethyl)amino]methyl}-2-phenylquinolin-4-yl)carbonyl]-1-phenylhydrazinecarboxylate;
methyl 2-[(3-{[benzyl(methyl)amino]methyl}-2-phenylquinolin-4-yl)carbonyl]-1-phenylhydrazinecarboxylate;
methyl 2-({3-[(4-methyl-3-oxopiperazin-1-yl)methyl]-2-phenylquinolin-4-yl}carbonyl)-1-phenylhydrazinecarboxylate;
methyl 2-[(3-{[4-(2-ethyl-4,5,6,7-tetrahydropyrazolo[1,5-a]pyridin-3-yl)piperidin-1-yl]methyl}-2-phenylquinolin-4-yl)carbonyl]-1-phenylhydrazinecarboxylate;
methyl 1-phenyl-2-[(2-phenyl-3-{[4-(2H-tetrazol-2-yl)piperidin-1-yl]methyl}quinolin-4-yl)carbonyl]hydrazinecarboxylate;
methyl 2-{[3-({4-[(1-methyl-1H-1,2,4-triazol-5-yl)methyl]-3-oxopiperazin-1-yl}methyl)-2-phenylquinolin-4-yl]carbonyl}-1-phenylhydrazinecarboxylate;
methyl 2-{[3-(5,6-dihydro[1,2,4]triazolo[4,3-a]pyrazin-7(8H)-ylmethyl)-2-phenylquinolin-4-yl]carbonyl}-1-phenylhydrazinecarboxylate;
methyl 2-[(3-{[3-(aminocarbonyl)piperidin-1-yl]methyl}-2-phenylquinolin-4-yl)carbonyl]-1-phenylhydrazinecarboxylate;
methyl 2-[(3-{[[2-(dimethylamino)ethyl](ethyl)amino]methyl}-2-phenylquinolin-4-yl)carbonyl]-1-phenylhydrazinecarboxylate;
methyl 2-[(3-{[(2R)-2-(aminocarbonyl)-1-pyrrolidinyl]methyl}-2-phenylquinolin-4-yl)carbonyl]-1-phenylhydrazinecarboxylate;
methyl 2-[(3-{[butyl(cyanomethyl)amino]methyl}-2-phenylquinolin-4-yl)carbonyl]-1-phenylhydrazinecarboxylate;
methyl 1-phenyl-2-({2-phenyl-3-[(2-pyridin-3-ylpyrrolidin-1-yl)methyl]quinolin-4-yl}carbonyl)hydrazinecarboxylate;
methyl 2-[(3-{[(2,3-dihydroxypropyl)(methyl)amino]methyl}-2-phenylquinolin-4-yl)carbonyl]-1-phenylhydrazinecarboxylate;
1-[(4-{[2-(methoxycarbonyl)-2-phenylhydrazino]carbonyl}-2-phenylquinolin-3-yl)methyl]piperidine-4-carboxylic acid;
methyl 2-{[3-(hexahydropyrrolo[1,2-a]pyrazin-2(1H)-ylmethyl)-2-phenylquinolin-4-yl]carbonyl}-1-phenylhydrazinecarboxylate;
methyl 1-phenyl-2-({2-phenyl-3-[(4-pyrimidin-2-ylpiperazin-1-yl)methyl]quinolin-4-yl}carbonyl)hydrazinecarboxylate;
methyl 2-({3-[(3-hydroxypiperidin-1-yl)methyl]-2-phenyl quinolin-4-yl}carbonyl)-1-phenylhydrazinecarboxylate;
(2S)-[[(4-{[2-(methoxycarbonyl)-2-phenylhydrazino]carbonyl}-2-phenylquinolin-3-yl)methyl](methyl)amino](phenyl)acetic acid;
1-[(4-{[2-(methoxycarbonyl)-2-phenylhydrazino]carbonyl}-2-phenylquinolin-3-yl)methyl]proline;
methyl 2-({3-[(4,4-difluoropiperidin-1-yl)methyl]-2-phenylquinolin-4-yl}carbonyl)-1-phenylhydrazinecarboxylate;
methyl 2-[(3-{[(2-hydroxy-2-phenylethyl)(methyl)amino]methyl}-2-phenylquinolin-4-yl)carbonyl]-1-phenylhydrazinecarboxylate;
methyl 2-{[3-({3-[acetyl(methyl)amino]pyrrolidin-1-yl}methyl)-2-phenylquinolin-4-yl]carbonyl}-1-phenylhydrazinecarboxylate;
methyl 2-[(3-{[4-(3-hydroxyphenyl)piperazin-1-yl]methyl}-2-phenylquinolin-4-yl)carbonyl]-1-phenylhydrazinecarboxylate;
methyl 2-[(3-{[4-(2-furoyl)piperazin-1-yl]methyl}-2-phenylquinolin-4-yl)carbonyl]-1-phenylhydrazinecarboxylate;
methyl 2-[(3-{[2-(hydroxymethyl)piperidin-1-yl]methyl}-2-phenylquinolin-4-yl)carbonyl]-1-phenylhydrazinecarboxylate;
methyl 2-[(3-{[(2S)-2-[(dimethylamino)carbonyl]-1-pyrrolidinyl]methyl}-2-phenylquinolin-4-yl)carbonyl]-1-phenylhydrazinecarboxylate;
methyl 2-({3-[(3-oxo-4-pyridin-4-ylpiperazin-1-yl)methyl]-2-phenylquinolin-4-yl}carbonyl)-1-phenylhydrazinecarboxylate;
methyl 1-phenyl-2-[(2-phenyl-3-{[3-(trifluoromethyl)-5,6-dihydro[1,2,4]triazolo[4,3-a]pyrazin-7(8H)-yl]methyl}quinolin-4-yl)carbonyl]hydrazinecarboxylate;
methyl 1-phenyl-2-[(2-phenyl-3-{[2-(trifluoromethyl)-5,6-dihydroimidazo[1,2-a]pyrazin-7(8H)-yl]methyl}quinolin-4-yl)carbonyl]hydrazinecarboxylate;
methyl 1-phenyl-2-[(2-phenyl-3-{[3-(piperidin-1-ylmethyl)piperidin-1-yl]methyl}quinolin-4-yl)carbonyl]hydrazinecarboxylate;
methyl 2-[(3-{[3-(3-ethyl-1,2,4-oxadiazol-5-yl)piperidin-1-yl]methyl}-2-phenylquinolin-4-yl)carbonyl]-1-phenylhydrazinecarboxylate;
methyl 2-[(3-{[methyl(2-thienylmethyl)amino]methyl}-2-phenylquinolin-4-yl)carbonyl]-1-phenylhydrazinecarboxylate;
methyl 2-({3-[(1-oxo-2-oxa-8-azaspiro[4.5]dec-8-yl)methyl]-2-phenylquinolin-4-yl}carbonyl)-1-phenylhydrazinecarboxylate;
methyl 2-[(3-{[3-(4-methyl-4H-1,2,4-triazol-3-yl)piperidin-1-yl]methyl}-2-phenylquinolin-4-yl)carbonyl]-1-phenylhydrazinecarboxylate;
methyl 1-phenyl-2-({2-phenyl-3-[(4-pyridin-4-ylpiperazin-1-yl)methyl]quinolin-4-yl}carbonyl)hydrazinecarboxylate;
methyl 2-[(3-{[4-(2-morpholin-4-yl-2-oxoethyl)piperazin-1-yl]methyl}-2-phenylquinolin-4-yl)carbonyl]-1-phenylhydrazinecarboxylate;
methyl 1-phenyl-2-[(2-phenyl-3-{[2-(trifluoromethyl)-5,6-dihydro[1,2,4]triazolo[1,5-a]pyrazin-7(8H)-yl]methyl}quinolin-4-yl)carbonyl]hydrazinecarboxylate;
tert-Butyl-4-[(4-{[2-(methoxycarbonyl)-2-phenylhydrazino]carbonyl}-2-phenylquinolin-3-yl)methyl]-2-oxopiperazin-1-carboxylate;
Methyl 1-phenyl-2-{[2-phenyl-3-(2-oxo-piperazin-1-ylmethyl)quinolin-4-yl]carbonyl}hydrazinecarboxylate;
Methyl 2-{[3-(benzyloxy)-2-phenylquinolin-4-yl]carbonyl}-1-phenylhydrazinecarboxylate;
Methyl 2-({3-[(1-methyl-1H-1,2,4-triazol-5-yl)methoxy]-2-phenylquinolin-4-yl}carbonyl)-1-phenylhydrazinecarboxylate;
Methyl 2-{[3-(cyanomethoxy)-2-phenylquinolin-4-yl]carbonyl}-1-phenylhydrazinecarboxylate;
Methyl-2-{[3-(2-methoxy-2-oxoethoxy)-2-phenylquinolin-4-yl]carbonyl}-1-phenylhydrazinecarboxylate;
[(4-{[2-(Methoxycarbonyl)-2-phenylhydrazino]carbonyl}-2-phenylquinolin-3-yl)oxy]acetic acid;
Methyl 2-({3-[(1-methyl-1H-imidazol-2-yl)methoxy]-2-phenylquinolin-4-yl}carbonyl)-1-phenylhydrazinecarboxylate;
Methyl 2-({3-[(1-methyl-1H-1,2,4-triazol-3-yl)methoxy]-2-phenylquinolin-4-yl}carbonyl)-1-phenylhydrazinecarboxylate;
2-(1,1-Dimethylethyl) 1-Methyl 2-[(3-Methyl-2-phenylquinolin-4-yl)carbonyl]-1-phenylhydrazinedicarboxylate;
2-(1,1-Dimethylethyl) 1-Methyl 2-{[3-(Bromomethyl)-2-phenylquinolin-4-yl]carbonyl}-1-phenylhydrazinedicarboxylate;
Methyl 2-{[3-(Bromomethyl)-2-phenylquinolin-4-yl]carbonyl}-1-phenylhydrazinecarboxylate;
2-(1,1-Dimethylethyl) 1-Methyl 2-[(8-Fluoro-3-methyl-2-phenylquinolin-4-yl)carbonyl]-1-phenylhydrazinedicarboxylate;
2-(1,1-Dimethylethyl) 1-Methyl 2-{[8-Fluoro-3-(bromomethyl)-2-phenylquinolin-4-yl]carbonyl}-1-phenylhydrazinedicarboxylate;
Methyl 2-{[8-Fluoro-3-(Bromomethyl)-2-phenylquinolin-4-yl]carbonyl}-1-Description 23: 5-Fluoro-2-(1,1-Dimethylethyl) 1-Methyl 2-[(5-Fluoro-3-methyl-2-phenylquinolin-4-yl)carbonyl]-1-phenylhydrazinedicarboxylate;
2-(1,1-Dimethylethyl) 1-Methyl 2-{[5-Fluoro-3-(bromomethyl)-2-phenylquinolin-4-yl]carbonyl}-1-phenylhydrazinedicarboxylate;
Methyl 2-{[5-Fluoro-3-(Bromomethyl)-2-phenylquinolin-4-yl]carbonyl}-1-phenylhydrazinecarboxylate;
2-(1,1-Dimethylethyl) 1-Methyl 2-[(7-Bromo-3-methyl-2-phenylquinolin-4-yl)carbonyl]-1-phenylhydrazinedicarboxylate;
2-(1,1-Dimethylethyl) 1-Methyl 2-{[7-Bromo-3-(bromomethyl)-2-phenylquinolin-4-yl]carbonyl}-1-phenylhydrazinedicarboxylate;
Methyl 2-{[7-Bromo-3-(Bromomethyl)-2-phenylquinolin-4-yl]carbonyl}-1-phenylhydrazinecarboxylate;
3-(5,6-Dihydro[1,2,4]triazolo[1,5-a]pyrazin-7(8H)-ylmethyl)-2-phenylquinoline-4-carboxylic Acid;
2-(1,1-Dimethylethyl) 1-Methyl 2-{[2-Phenyl-3-(2-propenyl)quinolin-4-yl]carbonyl}-1-phenylhydrazinedicarboxylate;
2-(1,1-Dimethylethyl) 1-Methyl 2-{[3-(2-Oxoethyl)-2-phenylquinolin-4-yl]carbonyl}-1-phenylhydrazinedicarboxylate;
2-(1,1-Dimethylethyl) 1-Methyl 2-[{3-[2-(4-Hydroxy)piperidinoethyl]-2-phenylquinolin-4-yl}carbonyl]-1-phenylhydrazinedicarboxylate;
Methyl 3-[1-(1,1-Dimethylethyl)piperidin-4-yloxy]-2-phenylquinoline-4-carboxylate;
3-[1-(1,1-Dimethylethyl)piperidin-4-yloxy]-2-phenylquinoline-4-carboxylic Acid;
Methyl 3-[1-(1,1-Dimethylethyloxycarbonyl)piperidin-4-yloxy]-2-phenylquinoline-4-carboxylate;
3-[1-(1,1-Dimethylethyloxycarbonyl)piperidin-4-yloxy]-2-phenylquinoline-4-carboxylic Acid;
1,1-Dimethylethyl 4-[(1E)-3-Oxo-3-phenyl-1-propenyl]-1-piperidinecarboxylate;
1,1-Dimethylethyl 4-(3-Oxo-3-phenyl-1-propyl-1-piperidinecarboxylate;
3-({1-[(1,1-Dimethylethoxy)carbonyl]piperidin-4-yl}methyl-2-phenylquinoline-4-carboxylic Acid;
3-({1-[(Phenylmethoxy)carbonyl]piperidin-4-yl}methyl)-2-phenylquinoline-4-carboxylic Acid;
3-({1-[(1,1-Dimethylethoxy)carbonyl]piperidin-4-yl}methyl)-N′,2-diphenylquinoline-4-carbohydrazide;
Methyl 2-{[3-({1-[(1,1-Dimethylethoxy)carbonyl]piperidin-4-yl}methyl)-2-phenylquinolin-4-yl]carbonyl}-1-phenylhydrazinecarboxylate;
3-({1-[(Phenylmethoxy)carbonyl]piperidin-4-yl}methyl)-N′,2-diphenylquinoline-4-carbohydrazide;
Methyl 2-{[3-({1-[(Phenylmethethoxy)carbonyl]piperidin-4-yl}methyl)-2-phenylquinolin-4-yl]carbonyl}-1-phenylhydrazinecarboxylate;
Methyl 2-({3-[(piperidin-4-yl)methyl]-2-phenylquinolin-4-yl}carbonyl)-1-phenylhydrazinecarboxylate;
methyl 2-[(3-{[(1S,4S)-5-(1,1-dimethylethyl)-2,5-diazabicyclo[2.2.1]hept-2-yl]methyl}-8-fluoro-2-phenylquinolin-4-yl)carbonyl]-1-phenylhydrazinecarboxylate;
methyl 2-({3-[(4-bicyclo[3.3.1]non-9-ylpiperazin-1-yl)methyl]-8-fluoro-2-phenylquinolin-4-yl}carbonyl)-1-phenylhydrazinecarboxylate;
methyl 2-({8-fluoro-3-[(4-isopropylpiperazin-1-yl)methyl]-2-phenylquinolin-4-yl}carbonyl)-1-phenylhydrazinecarboxylate;
methyl 2-({8-fluoro-3-[(4-oxohexahydropyrazino[2,1-c][1,4]oxazin-8(1H)-yl)methyl]-2-phenylquinolin-4-yl}carbonyl)-1-phenylhydrazinecarboxylate;
methyl 2-{[8-fluoro-3-(hexahydropyrrolo[1,2-a]pyrazin-2(1H)-ylmethyl)-2-phenylquinolin-4-yl]carbonyl}-1-phenylhydrazinecarboxylate;
methyl 2-{[8-fluoro-3-(hexahydropyrazino[2,1-c][1,4]oxazin-8(1H)-ylmethyl)-2-phenylquinolin-4-yl]carbonyl}-1-phenylhydrazinecarboxylate;
methyl 2-[(8-fluoro-3-{[4-(1-methylcyclohexyl)piperazin-1-yl]methyl}-2-phenylquinolin-4-yl)carbonyl]-1-phenylhydrazinecarboxylate;
methyl 2-[(3-{[3-(1-hydroxy-1-methylethyl)piperidin-1-yl]methyl}-2-phenylquinolin-4-yl)carbonyl]-1-phenylhydrazinecarboxylate;
methyl 2-({3-[(3-methyl-1,4′-bipiperidin-1′-yl)methyl]-2-phenylquinolin-4-yl}carbonyl)-1-phenylhydrazinecarboxylate;
methyl 2-[(3-{[4-(3-methyltetrahydro-2H-pyran-4-yl)piperazin-1-yl]methyl}-2-phenylquinolin-4-yl)carbonyl]-1-phenylhydrazinecarboxylate;
methyl 1-phenyl-2-[(2-phenyl-3-{[4-(tetrahydro-2H-pyran-3-yl)piperazin-1-yl]methyl}quinolin-4-yl)carbonyl]hydrazinecarboxylate;
methyl 2-({3-[(4-morpholin-4-ylpiperidin-1-yl)methyl]-2-phenylquinolin-4-yl}carbonyl)-1-phenylhydrazinecarboxylate;
methyl 1-phenyl-2-[(2-phenyl-3-{[4-(tetrahydro-2H-pyran-4-ylamino)piperidin-1-yl]methyl}quinolin-4-yl)carbonyl]hydrazinecarboxylate;
methyl 2-({3-[(4-hydroxy-1,4′-bipiperidin-1′-yl)methyl]-2-phenylquinolin-4-yl}carbonyl)-1-phenylhydrazinecarboxylate;
methyl 2-[(3-{[4-(1,1-dioxidotetrahydro-2H-thiopyran-4-yl)piperazin-1-yl]methyl}-2-phenylquinolin-4-yl)carbonyl]-1-phenylhydrazinecarboxylate;
methyl 1-phenyl-2-[(2-phenyl-3-{[4-(tetrahydro-2H-thiopyran-4-yl)piperazin-1-yl]methyl}quinolin-4-yl)carbonyl]hydrazinecarboxylate;
methyl 1-phenyl-2-[(2-phenyl-3-{[4-(2,2,2-trifluoroethyl)piperazin-1-yl]methyl}quinolin-4-yl)carbonyl]hydrazinecarboxylate;
2,5-anhydro-1,3,4-trideoxy-3-{4-[(4-{[2-(methoxycarbonyl)-2-phenylhydrazino]carbonyl}-2-phenylquinolin-3-yl)methyl]piperazin-1-yl}pentitol;
methyl 2-[(8-fluoro-2-phenyl-3-{[4-(tetrahydro-2H-pyran-4-yl)piperazin-1-yl]methyl}quinolin-4-yl)carbonyl]-1-phenylhydrazinecarboxylate;
methyl 2-[(8-fluoro-3-{[(7S)-7-hydroxyhexahydropyrrolo[1,2-a]pyrazin-2(1H)-yl]methyl}-2-phenylquinolin-4-yl)carbonyl]-1-phenylhydrazinecarboxylate;
methyl 2-[(8-fluoro-3-{[4-(4-methyltetrahydro-2H-pyran-4-yl)piperazin-1-yl]methyl}-2-phenylquinolin-4-yl)carbonyl]-1-phenylhydrazinecarboxylate;
methyl 2-{[3-(1,4-dioxa-8-azaspiro[4.5]dec-8-ylmethyl)-2-phenylquinolin-4-yl]carbonyl}-1-phenylhydrazinecarboxylate;
methyl 2-({3-[(4-cyclohexyl-3-oxopiperazin-1-yl)methyl]-2-phenylquinolin-4-yl}carbonyl)-1-phenylhydrazinecarboxylate;
methyl 2-[(3-{[4-(hydroxymethyl)piperidin-1-yl]methyl}-2-phenylquinolin-4-yl)carbonyl]-1-phenylhydrazinecarboxylate;
methyl 2-[(3-{[(2-cyanoethyl)(pyridin-3-ylmethyl)amino]methyl}-2-phenylquinolin-4-yl)carbonyl]-1-phenylhydrazinecarboxylate;
1,1-dimethylethyl 2-[(4-{[2-(methoxycarbonyl)-2-phenylhydrazino]carbonyl}-2-phenylquinolin-3-yl)methyl]-2,7-diazaspiro[4.5]decane-7-carboxylate;
methyl 2-{[3-(2,7-diazaspiro[4.5]dec-2-ylmethyl)-2-phenylquinolin-4-yl]carbonyl}-1-phenylhydrazinecarboxylate;
1,1-dimethylethyl (1R,4R)-5-[(4-{[2-(methoxycarbonyl)-2-phenylhydrazino]carbonyl}-2-phenylquinolin-3-yl)methyl]-2,5-diazabicyclo[2.2.1]heptane-2-carboxylate;
methyl 1-phenyl-2-{[2-phenyl-3-(1,4,6,7-tetrahydro-5H-pyrazolo[4,3-c]pyridin-5-ylmethyl)quinolin-4-yl]carbonyl}hydrazinecarboxylate;
methyl 2-{[3-(6,7-dihydrothieno[3,2-c]pyridin-5(4H)-ylmethyl)-2-phenylquinolin-4-yl]carbonyl}-1-phenylhydrazinecarboxylate;
methyl 1-phenyl-2-[(2-phenyl-3-{[2-(trifluoromethyl)-5,8-dihydro-1,7-naphthyridin-7(6H)-yl]methyl}quinolin-4-yl)carbonyl]hydrazinecarboxylate;
methyl 2-[(3-{[4-(2-oxo-2,3-dihydro-1H-benzimidazol-1-yl)piperidin-1-yl]methyl}-2-phenylquinolin-4-yl)carbonyl]-1-phenylhydrazinecarboxylate;
methyl 2-{[3-({4-[(2-methyl-1,3-thiazol-4-yl)methyl]piperazin-1-yl}methyl)-2-phenylquinolin-4-yl]carbonyl}-1-phenylhydrazinecarboxylate;
methyl 2-[(3-{[4-(2-oxopyrrolidin-1-yl)piperidin-1-yl]methyl}-2-phenylquinolin-4-yl)carbonyl]-1-phenylhydrazinecarboxylate;
methyl 2-({3-[(2-oxo-1-oxa-8-azaspiro[4.5]dec-8-yl)methyl]-2-phenylquinolin-4-yl}carbonyl)-1-phenylhydrazinecarboxylate;
methyl 2-({3-[(3-oxo-2,8-diazaspiro[4.5]dec-8-yl)methyl]-2-phenylquinolin-4-yl}carbonyl)-1-phenylhydrazinecarboxylate;
methyl 2-({3-[(3-methyl-2-oxo-1-oxa-3,8-diazaspiro[4.5]dec-8-yl)methyl]-2-phenylquinolin-4-yl}carbonyl)-1-phenylhydrazinecarboxylate;
methyl 2-({3-[(1-oxo-2,8-diazaspiro[4.5]dec-8-yl)methyl]-2-phenylquinolin-4-yl}carbonyl)-1-phenylhydrazinecarboxylate;
methyl 2-({3-[(2-methyl-1-oxo-2,8-diazaspiro[4.5]dec-8-yl)methyl]-2-phenylquinolin-4-yl}carbonyl)-1-phenylhydrazinecarboxylate;
methyl 2-{[3-(2-oxa-8-azaspiro[4.5]dec-8-ylmethyl)-2-phenylquinolin-4-yl]carbonyl}-1-phenylhydrazinecarboxylate;
methyl 2-({3-[(1,3-dioxo-2,8-diazaspiro[4.5]dec-8-yl)methyl]-2-phenylquinolin-4-yl}carbonyl)-1-phenylhydrazinecarboxylate;
methyl 1-phenyl-2-[(2-phenyl-3-{[3-(1H-1,2,4-triazol-1-yl)azetidin-1-yl]methyl}quinolin-4-yl)carbonyl]hydrazinecarboxylate;
methyl 1-phenyl-2-[(2-phenyl-3-{[2-(trifluoromethyl)-5,6-dihydro[1,2,4]triazolo[1,5-a]pyrazin-7(8H)-yl]methyl}quinolin-4-yl)carbonyl]hydrazinecarboxylate;
methyl 2-({3-[(1R,4R)-2,5-diazabicyclo[2.2.1]hept-2-ylmethyl]-2-phenylquinolin-4-yl}carbonyl)-1-phenylhydrazinecarboxylate;
methyl 2-({3-[(1-methyl-1,7-diazaspiro[4.4]non-7-yl)methyl]-2-phenylquinolin-4-yl}carbonyl)-1-phenylhydrazinecarboxylate;
methyl 2-({3-[(1-methyl-1,7-diazaspiro[4.4]non-7-yl)methyl]-2-phenylquinolin-4-yl}carbonyl)-1-phenylhydrazinecarboxylate;
1,1-dimethylethyl 7-[(4-{[2-(methoxy carbonyl)-2-phenylhydrazino]carbonyl}-2-phenylquinolin-3-yl)methyl]-2,7-diazaspiro[3.5]nonane-2-carboxylate;
methyl 2-[(3-{[(4R)-4-hydroxy-2-oxa-8-azaspiro[4.5]dec-8-yl]methyl}-2-phenylquinolin-4-yl)carbonyl]-1-phenylhydrazinecarboxylate;
methyl 2-[(3-{[(1S,4S)-5-methyl-2,5-diazabicyclo[2.2.1]hept-2-yl]methyl}-2-phenylquinolin-4-yl)carbonyl]-1-phenylhydrazinecarboxylate;
methyl 2-{[3-(2,3-dihydro-1H-pyrrolo[2,3-b]pyridin-1-ylmethyl)-2-phenylquinolin-4-yl]carbonyl}-1-phenylhydrazinecarboxylate;
1,1-dimethylethyl (1S,4S)-5-[(4-{[2-(methoxycarbonyl)-2-phenylhydrazino]carbonyl}-2-phenylquinolin-3-yl)methyl]-2,5-diazabicyclo[2.2.1]heptane-2-carboxylate;
methyl 2-({3-[(2-isopropyl-2,7-diazaspiro[3.5]non-7-yl)methyl]-2-phenylquinolin-4-yl}carbonyl)-1-phenylhydrazinecarboxylate;
methyl 2-[(3-{[4-(2-ethyl-1H-benzimidazol-1-yl)piperidin-1-yl]methyl}-2-phenylquinolin-4-yl)carbonyl]-1-phenylhydrazinecarboxylate;
methyl 2-[(3-{[(1R,4R)-5-isopropyl-2,5-diazabicyclo[2.2.1]hept-2-yl]methyl}-2-phenylquinolin-4-yl)carbonyl]-1-phenylhydrazinecarboxylate;
methyl 1-phenyl-2-[(2-phenyl-3-{[(1R,4R)-5-(3,3,3-trifluoropropyl)-2,5-diazabicyclo[2.2.1]hept-2-yl]methyl}quinolin-4-yl)carbonyl]hydrazinecarboxylate;
methyl 2-[(3-{[(1R,4R)-5-cyclohexyl-2,5-diazabicyclo[2.2.1]hept-2-yl]methyl}-2-phenylquinolin-4-yl)carbonyl]-1-phenylhydrazinecarboxylate;
methyl 1-phenyl-2-[(2-phenyl-3-{[(1R,4R)-5-(1,3-thiazol-2-ylmethyl)-2,5-diazabicyclo[2.2.1]hept-2-yl]methyl}quinolin-4-yl)carbonyl]hydrazinecarboxylate;
methyl 1-phenyl-2-[(2-phenyl-3-{[(1R,4R)-5-(tetrahydro-2H-pyran-4-yl)-2,5-diazabicyclo[2.2.1]hept-2-yl]methyl}quinolin-4-yl)carbonyl]hydrazinecarboxylate;
methyl 2-{[3-({(1R,4R)-5-[(5-chloro-2-thienyl)methyl]-2,5-diazabicyclo[2.2.1]hept-2-yl}methyl)-2-phenylquinolin-4-yl]carbonyl}-1-phenylhydrazinecarboxylate;
methyl 2-{[3-({(1R,4R)-5-[(5-chloro-2-furyl)methyl]-2,5-diazabicyclo[2.2.1]hept-2-yl}methyl)-2-phenylquinolin-4-yl]carbonyl}-1-phenylhydrazinecarboxylate;
methyl 2-{[3-({(1R,4R)-5-[(2-methyl-1H-imidazol-5-yl)methyl]-2,5-diazabicyclo[2.2.1]hept-2-yl}methyl)-2-phenylquinolin-4-yl]carbonyl}-1-phenylhydrazinecarboxylate;
methyl 2-({3-[((1R,4R)-5-{[5-(1,1-dimethylethyl)-1,2,4-oxadiazol-3-yl]methyl}-2,5-diazabicyclo[2.2.1]hept-2-yl)methyl]-2-phenylquinolin-4-yl}carbonyl)-1-phenylhydrazinecarboxylate;
methyl 2-({3-[(1S,4S)-2,5-diazabicyclo[2.2.1]hept-2-ylmethyl]-2-phenylquinolin-4-yl}carbonyl)-1-phenylhydrazinecarboxylate;
methyl 1-phenyl-2-[(2-phenyl-3-{[(1S,4S)-5-(tetrahydro-2H-pyran-4-yl)-2,5-diazabicyclo[2.2.1]hept-2-yl]methyl}quinolin-4-yl)carbonyl]hydrazinecarboxylate;
methyl 1-phenyl-2-[(2-phenyl-3-{[7-(tetrahydro-2H-pyran-4-yl)-2,7-diazaspiro[4.5]dec-2-yl]methyl}quinolin-4-yl)carbonyl]hydrazinecarboxylate;
methyl 1-phenyl-2-[(2-phenyl-3-{[7-(tetrahydro-2H-pyran-4-yl)-2,7-diazaspiro[4.4]non-2-yl]methyl}quinolin-4-yl)carbonyl]hydrazinecarboxylate;
methyl 2-{[3-(5,6-dihydro[1,2,4]triazolo[1,5-a]pyrazin-7(8H)-ylmethyl)-2-phenylquinolin-4-yl]carbonyl}-1-phenylhydrazinecarboxylate;
methyl 2-[(3-{[2-(1,1-dimethylethyl)-5,6-dihydro[1,2,4]triazolo[1,5-a]pyrazin-7(8H)-yl]methyl}-2-phenylquinolin-4-yl)carbonyl]-1-phenylhydrazinecarboxylate;
methyl 2-({3-[(2-methyl-5,6-dihydro[1,2,4]triazolo[1,5-a]pyrazin-7(8H)-yl)methyl]-2-phenylquinolin-4-yl}carbonyl)-1-phenylhydrazinecarboxylate;
methyl 2-{[3-(5,6-dihydro[1,2,4]triazolo[1,5-a]pyrazin-7(8H)-ylmethyl)-8-fluoro-2-phenylquinolin-4-yl]carbonyl}-1-phenylhydrazinecarboxylate;
methyl 2-{[3-(6,7-dihydro[1,2,3]triazolo[1,5-a]pyrazin-5(4H)-ylmethyl)-2-phenylquinolin-4-yl]carbonyl}-1-phenylhydrazinecarboxylate;
methyl 2-[(3-{[4-(1,1-dimethylethyl)piperazin-1-yl]methyl}-5-fluoro-2-phenylquinolin-4-yl)carbonyl]-1-phenylhydrazinecarboxylate;
methyl 2-[(3-{[3-(trifluoromethyl)piperazin-1-yl]methyl}-2-phenylquinolin-4-yl)carbonyl]-1-phenylhydrazinecarboxylate;
methyl 2-[(3-{[3-(trifluoromethyl)piperazin-1-yl]methyl}-5-fluoro-2-phenylquinolin-4-yl)carbonyl]-1-phenylhydrazinecarboxylate;
methyl 2-({3-[(1,1-dioxidothiomorpholin-4-yl)methyl]-2-phenylquinolin-4-yl}carbonyl)-1-phenylhydrazinecarboxylate;
methyl 2-({3-[(3-oxo-4-pyridin-2-ylpiperazin-1-yl)methyl]-2-phenylquinolin-4-yl}carbonyl)-1-phenylhydrazinecarboxylate;
methyl 2-[(3-{[4-(3-fluorophenyl)-3-oxopiperazin-1-yl]methyl}-2-phenylquinolin-4-yl)carbonyl]-1-phenylhydrazinecarboxylate;
methyl 2-{[3-({4-[1-(1,1-dimethylethyl)-3-methyl-1H-pyrazol-5-yl]piperidin-1-yl}methyl)-2-phenylquinolin-4-yl]carbonyl}-1-phenylhydrazinecarboxylate;
methyl 2-{[3-({4-[3-methyl-1-(2,2,2-trifluoroethyl)-1H-pyrazol-5-yl]piperidin-1-yl}methyl)-2-phenylquinolin-4-yl]carbonyl}-1-phenylhydrazinecarboxylate;
methyl 2-[(3-{[4-(4-methyl-1,2,5-oxadiazol-3-yl)piperazin-1-yl]methyl}-2-phenylquinolin-4-yl)carbonyl]-1-phenylhydrazinecarboxylate;
methyl 2-[(3-{[4-(1-ethyl-3-methyl-1H-pyrazol-5-yl)piperidin-1-yl]methyl}-2-phenylquinolin-4-yl)carbonyl]-1-phenylhydrazinecarboxylate;
methyl 2-({3-[(3-hydroxy-4,7-dihydroisoxazolo[5,4-c]pyridin-6(5H)-yl)methyl]-2-phenylquinolin-4-yl}carbonyl)-1-phenylhydrazinecarboxylate;
methyl 1-phenyl-2-[(2-phenyl-3-{[2-(trifluoromethyl)-5,8-dihydropyrido[3,4-d]pyrimidin-7(6H)-yl]methyl}quinolin-4-yl)carbonyl]hydrazinecarboxylate;
methyl 2-({3-[(3-ethyl-5,6-dihydro[1,2,4]triazolo[4,3-a]pyrazin-7(8H)-yl)methyl]-2-phenylquinolin-4-yl}carbonyl)-1-phenylhydrazinecarboxylate-methane (1:1);
methyl 2-({3-[(3-methoxy-6,7-dihydroisoxazolo[4,5-c]pyridin-5(4H)-yl)methyl]-2-phenylquinolin-4-yl}carbonyl)-1-phenylhydrazinecarboxylate;
methyl 2-[(3-{[4-(5-methoxypyrimidin-4-yl)piperazin-1-yl]methyl}-2-phenylquinolin-4-yl)carbonyl]-1-phenylhydrazinecarboxylate;
methyl 2-{[7-bromo-3-(5,6-dihydro[1,2,4]triazolo[1,5-a]pyrazin-7(8H)-ylmethyl)-2-phenylquinolin-4-yl]carbonyl}-1-phenylhydrazinecarboxylate;
methyl 2-[(7-bromo-3-{[4-(1,1-dimethylethyl)piperazin-1-yl]methyl}-2-phenylquinolin-4-yl)carbonyl]-1-phenylhydrazinecarboxylate;
methyl 2-{[3-(3′-hydroxyazetidnylmethyl)-2-phenylquinolin-4-yl]carbonyl}-1-phenylhydrazinecarboxylate;
methyl 2-{[3-(-(1-N(trifluoroethyl,1,2,3,triazolopiperidinyl[4,3-a]methyl)-2-phenylquinolin-4-yl]carbonyl}-1-phenylhydrazinecarboxylate?;
methyl 2-{[3-(1-N(methyl,1,2,3,triazolopiperidinyl[4,3-a]methyl)-2-phenylquinolin-4-yl]carbonyl}-1-phenylhydrazinecarboxylate?;
methyl 2-[(3-{[4-(1,1-dimethylethyl)piperazin-1-yl]methyl}-2-phenylquinolin-4-yl)carbonyl]-1-phenylhydrazinecarboxylate;
methyl 2-[(3-{[4-(2-fluoro-1,1-dimethylethyl)piperazin-1-yl]methyl}-2-phenylquinolin-4-yl)carbonyl]-1-phenylhydrazinecarboxylate;
Methyl 2-[(7-Cyano-3-{[4-(1,1-dimethylethyl)piperazin-1-yl]methyl}-2-phenylquinolin-4-yl)carbonyl]-1-phenylhydrazinecarboxylate;
Methyl 2-[(3-{[1-(2,3-dimethylcyclopentyl)piperazin-4-yl]methyl}-2-phenylquinolin-4-yl)carbonyl]-1-phenylhydrazinecarboxylate;
Methyl 2-[(3-{[1-(2,4-dimethylcyclopentyl)piperazin-4-yl]methyl}-2-phenylquinolin-4-yl)carbonyl]-1-phenylhydrazinecarboxylate;
Methyl 2-[(3-{[1-(2,4-dimethylcyclopentyl)piperazin-4-yl]methyl}-2-phenylquinolin-4-yl)carbonyl]-1-phenylhydrazinecarboxylate;
methyl 2-{[3-(N-(pyran-4-yl)-2-oxopiperazinylmethyl)-2-phenylquinolin-4-yl]carbonyl}-1-phenylhydrazinecarboxylate;
methyl 2-{[3-(-(N-(3′-ethylcyclopentyl)-2-oxopiperazinylmethyl)-)-2-phenylquinolin-4-yl]carbonyl}-1-phenylhydrazinecarboxylate;
Methyl 2-{[2-Phenyl-3-(1H-1,2,4-triazol-1-ylmethyl)quinolin-4-yl]carbonyl}-1-phenylhydrazinecarboxylate;
Methyl 2-{[3-(1H-Benzimidazol-1-ylmethyl)-2-phenylquinolin-4-yl]carbonyl}-1-phenylhydrazinecarboxylate;
Methyl 2-([2-Phenyl-3-(1H-tetrazol-1-ylmethyl)quinolin-4-yl]carbonyl)-1-phenylhydrazinecarboxylate and Methyl 2-([2-Phenyl-3-(2H-tetrazol-2-ylmethyl)quinolin-4-yl]carbonyl)-1-phenylhydrazinecarboxylate;
Methyl 2-[(3-{[3-(Methoxycarbonyl)-1H-1,2,4-triazol-1-yl]methyl}-2-phenylquinolin-4-yl)carbonyl]-1-phenylhydrazinecarboxylate;
Methyl 1-phenyl-2-({2-phenyl-3-[(4-pyridin-2-yl-1H-1,2,3-triazol-1-yl)methyl]quinolin-4-yl}carbonyl)hydrazinecarboxylate;
methyl 1-[(4-{[2-(methoxycarbonyl)-2-phenylhydrazino]carbonyl}-2-phenylquinolin-3-yl)methyl]-1H-1,2,3-triazole-4-carboxylate;
methyl 1-phenyl-2-({2-phenyl-3-[(4-pyridin-3-yl-1H-1,2,3-triazol-1-yl)methyl]quinolin-4-yl}carbonyl)hydrazinecarboxylate;
methyl 2-({3-[(4-acetyl-1H-1,2,3-triazol-1-yl)methyl]-2-phenylquinolin-4-yl}carbonyl)-1-phenylhydrazinecarboxylate;
methyl 2-{[3-({4-[(1,1-dioxidothiomorpholin-4-yl)methyl]-1H-1,2,3-triazol-1-yl}methyl)-2-phenylquinolin-4-yl]carbonyl}-1-phenylhydrazinecarboxylate;
Methyl 2-{[3-(4′-Hydroxypiperidinylethyl)-2-phenylquinolin-4-yl]carbonyl}-1-phenylhydrazinecarboxylate;
Methyl 2-{[3-(2-Hydroxyethoxy)-2-phenylquinolin-4-yl]carbonyl}-1-phenylhydrazinecarboxylate;
Methyl 2-{[3-(2-Bromoethoxy)-2-phenylquinolin-4-yl]carbonyl}-1-phenylhydrazinecarboxylate;
3-[1-(1,1-Dimethylethyl)piperidin-4-yloxy]-N′,2-diphenylquinoline-4-carbohydrazide;
Methyl 2-({3-[1-(1,1-dimethylethyl)piperidin-4-yloxy]-2-phenylquinolin-4-yl}carbonyl)-1-phenylhydrazinecarboxylate;
3-[1-(1,1-Dimethylethyloxycarbonyl)piperidin-4-yloxy]-N′,2-diphenylquinoline-4-carbohydrazide;
Methyl 2-({3-[1-(1,1-Dimethylethyloxycarbonyl)piperidin-4-yloxy]-2-phenylquinolin-4-yl}carbonyl)-1-phenylhydrazinecarboxylate;
Methyl 2-({2-Phenyl-3-(piperidin-4-yloxy)quinolin-4-yl}carbonyl)-1-phenylhydrazinecarboxylate;
Methyl 2-[(3-(4-oxo-1-(4-pyranyl)-piperidinyl)-2-phenylquinolin-4-yl)carbonyl]-1-phenylhydrazinecarboxylate;
Methyl 1-phenyl-2-{[2-phenyl-3-({[1-(tetrahydro-2H-pyran-4-yl)azetidin-3-yl]oxy}methyl)quinolin-4-yl]carbonyl}hydrazinecarboxylate;
Methyl 1-phenyl-2-({2-Phenyl-3-[(4-piperidinyloxy)methyl]quinolin-4-yl}carbonyl)hydrazinecarboxylate;
Methyl 1-phenyl-2-{[2-phenyl-3-({[1-(tetrahydro-2H-pyran-4-yl)-4-piperidinyl]oxy}methyl)quinolin-4-yl]carbonyl}hydrazinecarboxylate;
Methyl 2-({3-[(1-Ethyl-1H-1,2,4-triazol-5-yl)methoxymethyl]-2-phenylquinolin-4-yl}carbonyl)-1-phenylhydrazinecarboxylate;
Methyl 2-{[3-(Methylthiomethyl)-2-phenylquinolin-4-yl]carbonyl}-1-phenylhydrazinecarboxylate;
Methyl 2-{[3-(Methylthiosulfonylmethyl)-2-phenylquinolin-4-yl]carbonyl}-1-phenylhydrazinecarboxylate;
Methyl 2-{[3-(Methylsulfoxidylmethyl)-2-phenylquinolin-4-yl]carbonyl}-1-phenylhydrazinecarboxylate;
Methyl 2-[(3-{[1-(Pyran-4-yl)piperidin-4-yl]methyl}-2-phenylquinolin-4-yl)carbonyl]-1-phenylhydrazinecarboxylate;
3-(5,6-dihydro[1,2,4]triazolo[1,5-a]pyrazin-7(8H)-ylmethyl)-N′,N′,2-triphenylquinoline-4-carbohydrazide;
3-(5,6-dihydro[1,2,4]triazolo[1,5-a]pyrazin-7(8H)-ylmethyl)-N′-(3-fluorophenyl)-N′,2-diphenylquinoline-4-carbohydrazide;
3-(5,6-dihydro[1,2,4]triazolo[1,5-a]pyrazin-7(8H)-ylmethyl)-N′,N′-bis(3-fluorophenyl)-2-phenylquinoline-4-carbohydrazide;
3-(5,6-dihydro[1,2,4]triazolo[1,5-a]pyrazin-7(8H)-ylmethyl)-N′-ethyl-N′,2-diphenylquinoline-4-carbohydrazide;
3-(5,6-dihydro[1,2,4]triazolo[1,5-a]pyrazin-7(8H)-ylmethyl)-N′,2-diphenyl-N′-pyridin-3-ylquinoline-4-carbohydrazide;
3-(5,6-dihydro[1,2,4]triazolo[1,5-a]pyrazin-7(8H)-ylmethyl)-N′,2-diphenyl-N′-1,3-thiazol-2-ylquinoline-4-carbohydrazide;
1,1-Dimethylethyl 4-[(2-Phenyl-4-({2,2-diphenylhydrazino}carbonyl)quinolin-3-yl)methyl]piperidine-1-carboxylate;
or a pharmaceutically acceptable salt thereof.

30. A pharmaceutical composition comprising the compound of claim 18 or a pharmaceutically acceptable salt thereof and a pharmaceutically acceptable excipient.

31. A method for the treatment of a subject suffering from a neurokinin-2 and/or neurokinin-3 mediated disease, which comprises administering to that patient a therapeutically effective amount of the compound of claim 18 or a pharmaceutically acceptable salt thereof.

32. The method of claim 31 wherein the neurokinin-2 and/or neurokinin 3 mediated disease is selected from the group consisting of: depression; an anxiety disorder; a phobia; psychosis; psychotic disorders; post-traumatic stress disorder; attention deficit hyperactive disorder (ADHD); withdrawal from abuse of drugs; smoking cessation; reduction in level or frequency of abuse of drugs; reduction in level or frequency of smoking; and irritable bowel syndrome.
